(19) United States
(12) Patent Application Publication
(10) Pub. No.: US 2016/0313303 A1
(43) Pub. Date: Oct. 27, 2016

(54) BICOMPATIBLE LIQUID AND METHOD FOR SCREENING SAME

(71) Applicant: KABUSHIKI KAISHA TOYOTA CHUO KENKYUSHO, Nagakute-shi (JP)

(72) Inventors: Minoru HIRANO, Nagakuta-shi (JP); Daisuke NAKAMURA, Nagakute-shi (JP); Riichiro OHTA, Nagakute-shi (JP)

(73) Assignee: KABUSHIKI KAISHA TOYOTA CHUO KENKYUSHO, Nagakute-shi (JP)

(21) Appl. No.: 15/079,871

(22) Filed: Mar. 24, 2016

(30) Foreign Application Priority Data
Mar. 27, 2015 (JP) 2015-067598
Mar. 27, 2015 (JP) 2015-067599

(51) Int. Cl. G01N 33/50

(52) U.S. Cl. CPC G01N 33/5014; G01N 33/502

(57) ABSTRACT

An object of the present invention is to provide a biocompatible liquid that is more essential and practical, and a method for screening the same.

The object is achieved by using, as the biocompatible liquid, a liquid having a Hansen solubility parameter (HSP) compatible to a cell to which the liquid is applied.

### TECHNICAL FIELD

The present application claims priority to Japanese Patent Applications No, 2015-67598 filed on Mar. 27, 2015 and No. 2015-67599 filed on Mar. 27, 2015, the contents of which are hereby incorporated by reference into the present application.

The present specification relates to a liquid that is compatible with living organisms, a method for determining information on a threshold for identifying the liquid, a method for screening the liquid, and the like.

### DESCRIPTION OF RELATED ART

Conventionally, toxicity of various compounds to living organisms such as animals including humans and plants has been evaluated by directly administering the compounds to individual organisms or by a method using a wide range of cells and the like. For example, toxicity of compounds and the like to living organisms has been conventionally and generally evaluated by dissolving or dispersing the compounds in a liquid such as water or buffers and then bringing the liquid into contact with cells or the like for cultivation. The reason for using water or buffers is that all organisms including humans are made up of water. Various evaluation methods of the above type have already been known (Non-patent document 1). Thus, cytotoxicity has been generally evaluated by using water as a liquid and a test compound as a solute.

### Non-Patent Document

[Non-patent document 1] Toxicol Ind Health. 2006 August; 22 (7): 301-15

### BRIEF SUMMARY

Cells in vivo are generally protected from the outside world by dead cells and corneum or mucous membranes which are mixtures of secreted proteins, sebum and the like. However, cells per se are essentially in contact with the outside world only through cell membranes without such extracellular components (hereinafter these cells are referred to as naked cells). Examples of naked cells include cultured cells and cells exposed by surgery and the like.

Meanwhile, pure water is safe for skin, bronchial epithelia and the like. However, pure water is highly toxic for naked cells because of low osmotic pressure thereof resulting in death of the cells in a few seconds. Thus when cytotoxicity and biocompatibility are considered in terms of essential significance, it is essentially problematic to envision pure water as liquid used for evaluation. Therefore, pure water is not appropriate as liquid for evaluating cytotoxicity and biocompatibility.

On the other hand, organic solvents are actually regarded as generally cytotoxic. Therefore, there is no knowledge as to which liquid is biocompatible in tens of essential significance. Also, no method for screening the liquid has yet been presented. Further, no method or device for appropriately evaluating the action of a nonhydrophilic substance on cells has been presented so far.

The present specification provides a biocompatible liquid that is more essential and practical, and a method for the same. The present specification also provides a structure for exposing cells to a non-hydrophilic substance an a method for evaluating an action of a non-hydrophilic substance on cells.

The inventors of the present invention carried out various investigations by using cells on liquids such as organic solvents and biotoxicity thereof. As a result, the inventors conceived a technical idea as to organic solvents and biocompatibility thereof that has not conventionally occurred to anyone. Thus, the inventors of the present invention re-constructed the concept on biocompatibility to naked cells that are isolated from the outside world only through cell membranes which are components thereof, namely are not protected by corneum, mucous membranes or the like and thus are more sensitive as cells. As a result, the inventors found that some organic solvents have high biocompatibility without cytotoxicity even when the solvents are used of their own, in other word, at 100% of the concentration.

The inventors of the present invention found that, as a result of evaluations of biocompatible liquids from the viewpoint of the above, organic solvents fulfilling a certain parameter may be biocompatible. Thus, the inventors found the relationship between a parameter of organic solvents and biocompatibility. On the basis of the finding, the present specification provides the following measures.

[1]A biocompatible liquid having a Hansen solubility parameter (HSP) compatible to a cell to which the liquid is applied.
[2] The liquid according to [1], wherein the compatible HSP is determined on the basis of threshold information of a HSP associated with biocompatibility obtained on the basis of one or more liquids far which a level of biocompatibility to the cell has been established and HSPs of the liquids.
[3] The liquid according to [1] or [2], wherein the compatible HSP is present within a HSP sphere defined by a predetermined core (δD, δP, δH) in a HSP space based on HSPs of one or more liquids biocompatible to the cell and by a predetermined interaction radius R.
[4] The liquid according to any of [1] to [3], wherein the compatible HSP is present outside of a HSP sphere defined by a predetermined core (δD, δP, δH) in a HSP space based on HSPs of one or more cell components of the cell and by a predetermined interaction radius.
[5] The liquid according to any of [1] to [4], having a molar volume compatible to the cell.
[6] The liquid according to [5], wherein the compatible molar volume is determined on the basis of threshold information of a liquid molar volume associated with biocompatibility obtained on the basis of one or more liquids for which a level of biocompatibility to the cell has been established and molar volumes of the liquids.
[7] The liquid according to any of [1] to [3], wherein:

a molar volume is less than 330 cm³/mol; and

the HSP is present within a HSP sphere having a core (δD, δP, δH) of (12.73, 2.33 3.46) ([J/cm³]^{1/2}) and an interaction radius R of 3.4 ([J/cm³]^{1/2}).

[8] The liquid according to any of [1] to [3], wherein:

a liquid molar volume is 330 cm³/mol or more; and

the HSP is present within a HSP sphere having a core (δD, δP, δH) of (12.73, 2.33, 3.46) ([J/cm³]^{1/2}) and an interaction radius R of 9.0 ([J/cm³]^{1/2}).

[9] The liquid according to any of [1], [2] and [4], wherein:

a liquid molar volume is 125 cm³/mol or more; and

the HSP is present outside of all HSP spheres of DNA, cholesterol, water, phosphatidylcholine, sphingomyelin, phosphatidylethanolamine and phosphatidylserine.

[10] The liquid according to any of [1] to [9], wherein the liquid has a boiling point of above 33° C. and a melting point of less than 25° C.
[11] The liquid according to any of [1] to [10], which is one or more liquids selected from the following group:

TABLE 1

| ID | Solvent | CAS No. | SMILES or Formula |
|---|---|---|---|
| S5 | HFE-7100 | 163702-07-6 | COC(C(C(C(F)(F)F)(F)F)(F)F)(F)F |
| S6 | HFE-7200 | 163702-06-5, | CCOC(F)(F)C(F)(F)C(F)(F)C(F)(F)(F), |
| | | 163702-05-4 | CCOC(F)(F)C(C(F)(F)(F))(F)C(F)(F)(F) |
| S7 | HFE-7300 | 132182-92-4 | FC(F)(C(F)(F)C(F)(F)C(F)(F)C(F)(F)F)C(F)(F)OC |
| S8 | GALDEN HT55 | Mixture | CF3—[(O—CF—CF2)1—(O—CF2)0.3]—O—CF3 |
| S9 | GALDEN HT80 | Mixture | CF3—[(O—CF—CF2)1.17—(O—CF2)1.24]—O—CF3 |
| S11 | GALDEN HT135 | Mixture | CF3—[(O—CF—CF2)2.21—(O—CF2)1.36]—O—CF3 |
| S12 | GALDEN HT170 | Mixture | CF3—[(O—CF—CF2)3—(O—CF2)1.63]—O—CF3 |
| S13 | GALDEN HT200 | Mixture | CF3—[(O—CF—CF2)4—(O—CF2)0.79]—O—CF3 |
| S14 | GALDEN HT230 | Mixture | CF3—[(O—CF—CF2)4—(O—CF2)3.06]—O—CF3 |
| S18 | OS-30 (Decamethyltetrasil [symbol] | 141-62-8 | C[Si](C)(C)O[Si](C)(C)O[Si](C)(C)O[Si](C)(C)C |
| S19 | Decamethylcyclopentasilox [symbol] | 541-02-6 | [Si]1(C)(C)O[Si](C)(C)O[Si](C)(C)O[Si](C)(C)O[Si](O1)(C)C |
| S20 | 1-Bromoperfluorooctane | 423-55-2 | FC(F)(F)C(F)(F)C(F)(F)C(F)(F)C(F)(F)C(F)(F)C(F)(F)C(F)(F)Br |
| S21 | HFE-7000 | 375-03-1 | COC(F)(C(F)(C(F)(F)F)F)F |

[symbol]

indicates data missing or illegible when filed

[12] The liquid according to any of [1] to [1], which is biocompatible to a naked cell devoid of an extracellular component.
[13]. A method for determining HSP threshold information for identifying a biocompatible liquid, comprising the steps of:

obtaining biocompatibility and a HSP of one or more liquids to a cell to which the liquids are applied; and

defining, on the basis of the biocompatibility and the HSP, a HSP sphere serving as the HSP threshold information by a core (δD, δP, δH) in a HSP space associated with predetermined biocompatibility a and a interaction radius R.

[4] The method according to [13], further comprising a step of further obtaining a molar volume of the one or more liquids, wherein

the HSP sphere is defined on the basis of the molar volume.

[15]A method for determining HSP threshold information for identifying a biocompatible liquid, comprising the steps of:

obtaining, for one or more liquids, a HSP of a cell component of a cell to which the liquids are applied; and

defining, on the basis of the HSP, a HSP sphere serving as the HSP threshold information by a core (δD, δP, δH) of the cell component in a HSP sphere and an interaction radius R.

[16]A method for screening a biocompatible liquid, comprising:

a step of identifying whether or not a test liquid has a HSP compatible to a cell to which the liquid is applied and/or a step of identifying whether or not the liquid has a molar volume compatible to the cell to which the liquid is applied.

[17]A cell-containing structure comprising:

a first liquid carrier through which a first liquid that is a hydrophilic liquid can flow or which can retain the first liquid;

a second liquid carrier through which a second liquid that is a non-hydrophilic liquid immiscible with the first liquid and contains a non-hydrophilic substance can flow or which can retain the second liquid;

a support through which either or both of the first liquid and the second liquid can move; and

a cell retained in at least a part of the support while contacting the first liquid and the second liquid.

[18] A method for evaluating an action of a non-hydrophilic substance on a cell, comprising the steps of:

culturing the cell while the cell is in contact with a first liquid that is a hydrophilic liquid, and a second liquid that is a non-hydrophilic liquid immiscible with the first liquid and contains the non-hydrophilic substance, by using a support through which either or both of the first liquid and the second liquid can move as a scaffold in the vicinity of an interface between the first liquid and the second liquid; and

evaluating the action of the non-hydrophilic substance on the cell.

[19] The structure according to [18], the non-hydrophilic liquid is selected from the following liquids.

TABLE 2

| ID | Solvent | CAS No. | SMILES or Formula |
|---|---|---|---|
| S5 | HFE-7100 | 163702-07-6 | COC(C(C(C(F)(F)F)(F)F)(F)F)(F)F |
| S6 | HFE-7200 | 163702-06-5, | CCOC(F)(F)C(F)(F)C(F)(F)C(F)(F)(F), |
| | | 163702-05-4 | CCOC(F)(F)C(C(F)(F)(F))(F)C(F)(F)(F) |
| S7 | HFE-7300 | 132182-92-4 | FC(F)(C(F)(F)C(F)(F)C(F)(F)C(F)(F)F)C(F)(F)OC |
| S8 | GALDEN HT55 | Mixture | CF3—[(O—CF—CF2)1—(O—CF2)0.3]—O—CF3 |
| S9 | GALDEN HT80 | Mixture | CF3—[(O—CF—CF2)1.17—(O—CF2)1.24]—O—CF3 |
| S11 | GALDEN HT135 | Mixture | CF3—[(O—CF—CF2)2.21—(O—CF2)1.36]—O—CF3 |
| S12 | GALDEN HT170 | Mixture | CF3—[(O—CF—CF2)3—(O—CF2)1.63]—O—CF3 |
| S13 | GALDEN HT200 | Mixture | CF3—[(O—CF—CF2)4—(O—CF2)0.79]—O—CF3 |
| S14 | GALDEN HT230 | Mixture | CF3—[(O—CF—CF2)4—(O—CF2)3.06]—O—CF3 |
| S18 | OS-30 (Decamethyltetrasil [symbol] | 141-62-8 | C[Si](C)(C)O[Si](C)(C)O[Si](C)(C)O[Si](C)(C)C |
| S19 | Decamethylcyclopentasilox [symbol] | 541-02-6 | [Si]1(C)(C)O[Si](C)(C)O[Si](C)(C)O[Si](C)(C)O[Si](O1)(C)C |
| S20 | 1-Bromoperfluorooctane | 423-55-2 | FC(F)(F)C(F)(F)C(F)(F)C(F)(F)C(F)(F)C(F)(F)C(F)(F)C(F)(F)Br |
| S21 | HFE-7000 | 375-03-1 | COC(F)(C(F)(C(F)(F)F)F)F |

[symbol]

indicates data missing or illegible when filed

[20]A method for screening a cytocompatible non-hydrophilic substance, comprising the steps of:

culturing a cell while the cell is in contact with a first liquid that is a hydrophilic liquid, and a second liquid that is a non-hydrophilic liquid that is a non-hydrophilic liquid immiscible with the first liquid and contains the non-hydrophilic substance, by using a support through which either or both of the first liquid and the second liquid can move as a scaffold at an interface between the first liquid and the second liquid, and

evaluating an action of the non-hydrophilic substance on the cell,

wherein the cytocompatibility of the non-hydrophilic substance is evaluated on the basis of the action.

### BRIEF DESCRIPTION OF DRAWINGS

FIG. 1 shows the relationship between biocompatible liquids according to the present disclosure and HSPs;

FIG. 2 shows the relationship between molar volumes of test liquids and normalized cell survival rates;

FIG. 3 shows an HSP space representing HSPs of test liquids having a molar volume of less than 330 cm³/mol and a normalized cell survival rate of 0.7 or more, a HSP core based on the HSPs and HSPs of cytotoxic liquids;

FIG. 4 is a plot of the normalized cell survival rates and the distances D from the HSP core for test liquids having a molar volume of less than 330 cm³/mol;

FIG. 5 is a plot of the normalized cell survival rates and the distances D from the HSP core for test liquids having a molar volume of 330 cm³/mol or more;

FIG. 6 shows the relationship between HSPs of biocompatible liquids and HSP spheres of cell components;

FIG. 7 shows an example of the concept of the evaluation of a non-hydrophilic substance of the present disclosure;

FIG. 8 shows a first embodiment of the evaluation of a non-hydrophilic substance of the present disclosure;

FIG. 9 shows a second embodiment of the evaluation of a non-hydrophilic substance of the present disclosure;

FIG. 10 shows a modification of the second embodiment of the evaluation of a non-hydrophilic substance of the present disclosure;

FIG. 11 shows a modification of the second embodiment of the evaluation of a non-hydrophilic substance of the present disclosure;

FIG. 12 shows a third embodiment of the evaluation of a non-hydrophilic substance of the present disclosure;

FIG. 13 shows a fourth embodiment of the evaluation of a non-hydrophilic substance of the present disclosure;

FIG. 14 shows a modification of the third embodiment of the evaluation of a non-hydrophilic substance of the present disclosure;

FIG. 15 shows a modification of the fourth embodiment of the evaluation of a non-hydrophilic substance of the present disclosure;

FIG. 16 shows an evaluation device used in Examples;

FIG. 17 shows an embodiment of the cultivation step in Examples;

FIG. 18 shows the results of evaluation (ER) of the action of perfluorohexane and two hydrofluoroethers on cells;

FIG. 19 shows the results of evaluation (absorbance) of cytocompatibility of solutions of perfluorooctanoic acid (PFOA) in hydrofluoroether; and

FIG. 20 shows the result of evaluation (relative absorbance) of cytocompatibility of solutions of perfluorooctanoic acid (PFOA) in hydrofluoroether.

### DETAILED DESCRIPTION OF INVENTION

The present disclosure relates to a biocompatible liquid, a method for determining an index for a biocompatible liquid and a method for screening a biocompatible liquid and a method for screening a biocompatible liquid. The present disclosure relates to the finding that Hansen solubility parameters (HSPs) of liquids are a powerful index of biocompatibility.

“Cells” in the context of cytotoxicity may be essentially naked cells that are not provided with extracellular components. In view of this, main causes of cytotoxicity may include (1) lysis of cell membranes by a liquid; (2) disruption of cell metabolism due to diffusion and infiltration of a liquid into cells resulting in reaction with and denaturation of biological components; and (3) damage on DNA.

From the above idea, the inventors of the present invention re-evaluated various liquids including organic solvents to successfully obtain a novel index.

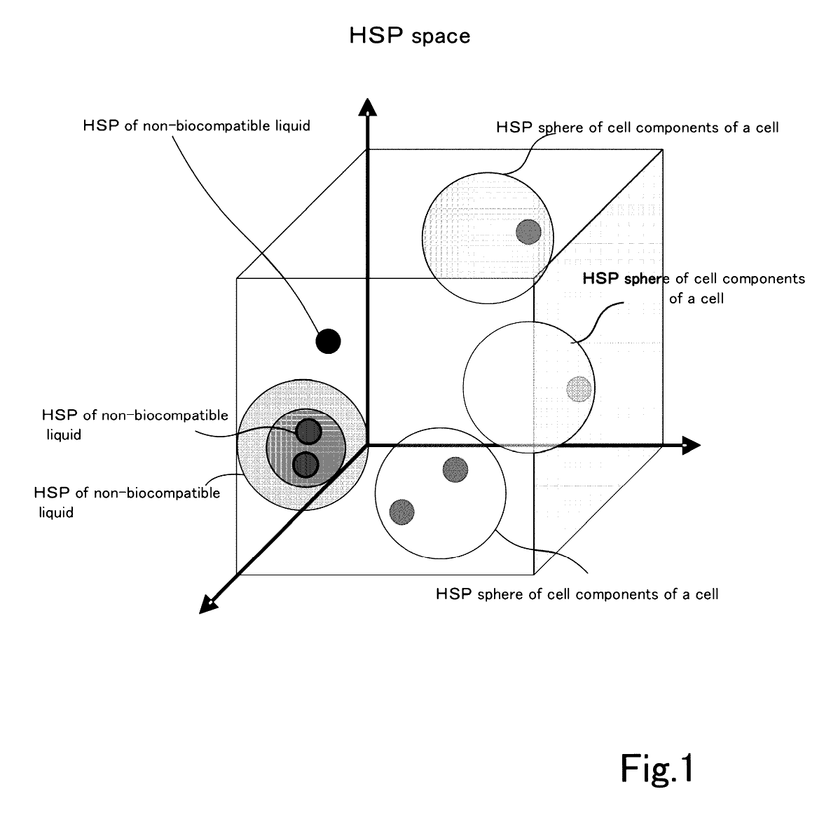

FIG. 1 shows an example of possible HSPs of biocompatible liquids. Certain biocompatible liquids may have HSPs that are within a specific HSP sphere. Certain biocompatible liquids may have HSPs outside of an HSP sphere of a cell component.

With regard to the biocompatibility of liquids, molar volume is taken into account. The molar volume represents the volume that may be occupied by one mole of a liquid. When a liquid has an increased molar volume, the liquid has less capability of passing through the cell membrane, and thus has an improved biocompatibility to naked cells. On the other hand, when a liquid has a decreased molar volume, the liquid has an increased capability of passing through the cell membrane, and thus has decreased biocompatibility to naked cells.

Accordingly, a liquid of which HSP is outside of an HSP sphere of a cell component, for example, does not interact with, e.g. dissolve, infiltrate or damage, the cell component, and thus may exhibit biocompatibility. When a liquid has an increased molar volume, the liquid has decreased interaction with, e.g. dissolution, infiltration and damaging of, the cell component, and thus the radius of the HSP sphere of the liquid may be increased.

As described above, according to the present disclose, biocompatibility of a liquid may be identified on the basis of the HSP thereof and a certain HSP sphere and further the molar volume thereof. In other words, by selecting a liquid having a certain HSP and molar volume, a biocompatible liquid can be efficiently screened.

The cell-containing structure disclosed in the present specification may include a first liquid carrier through which a first liquid that is a hydrophilic liquid can flow or which can retain the first liquid; a second liquid carrier through which a second liquid that is a non-hydrophilic liquid immiscible with the first liquid and contains a non-hydrophilic substance can flow or which can retain the second liquid; a support through which either or both of the first liquid and the second liquid can move; and a cell retained in at least a part of the support while contacting the first liquid and the second liquid.

According to the present disclosure, cells are retained in a predetermined support provided at an interface between a first liquid that is a hydrophilic liquid, and a second liquid that is a non-hydrophilic liquid containing a non-hydrophilic substance. Thus, the cells can grow while contacting the first liquid that is a hydrophilic liquid, and the second liquid that is a non-hydrophilic liquid containing a non-hydrophilic substance. Namely, the cells retained in the support can be supplied with nutritional components or the like via the first liquid while being exposed to the second liquid which may affect the cells.

The above situation mimics or reproduces the contact of cells to a non-hydrophilic substance which is a foreign substance in vivo. Because the situation of contact can be secured, the present invention allows maintenance of exposure to a non-hydrophilic substance for a prolonged period of time.

Thus, according to the present disclosure, a practical method for evaluating an action of a non-hydrophilic substance on cells that allows evaluation of the non-hydrophilic substance with high accuracy can be provided. As a result, according to the present disclosure, cytocompatibility or cytotoxicity of a non-hydrophilic substance may be evaluated in terms of essential significance. Further, according to the present disclosure, a structure can be provided that is suitable for evaluating an action of a non-hydrophilic substance and that is for exposing cells to the non-hydrophilic substance.

The term “hydrophilic liquid” as used in the present specification means a liquid that possesses hydrophilicity towards water and is miscible with water. A liquid may be referred to as a hydrophilic liquid when liquid when the liquid is miscible with water at a temperature of 0° C. or higher and 70° C. or lower, preferably 0° C. or higher and 60° C. or lower, more preferably 0° C. or higher and 50° C. or lower and still more preferably 0° C. or higher and 40° C. or lower, regardless of the ratio thereof to water. The hydrophilic liquid is preferably freely miscible with water. The hydrophilic liquid is in a liquid state under a temperature at which the liquid is miscible with water.

Examples of the hydrophilic liquid include water, an organic solvent miscible with water and a mixed solution of two or more of the foregoings. The organic solvent miscible with water typically includes, but is not limited to, lower alcohols such as methanol, ethanol, n-propyl alcohol, isopropyl alcohol, n-butyl alcohol, isobutyl alcohol and tert-butyl alcohol, acetone, acetonitrile, dimethylformamide, dimethyl sulfoxide and the like.

The term “non-hydrophilic liquid” as used herein means a liquid that is immiscible with the hydrophilic liquid described above. A liquid may be referred to as a non-hydrophilic liquid when the liquid is immiscible with water at a temperature of 0° C. or higher and 70° C. or lower, preferably 0° C. or higher and 60° C. or lower, more preferably 0° C. or higher and 50° C. or lower and still more preferably 0° C. or higher and 40° C. or lower, regardless of the ratio thereof to water. The non-hydrophilic liquid preferably forms a phase separation with water. The non-hydrophilic liquid is in a liquid state under a temperature at which the liquid is immiscible with water.

Examples of the non-hydrophilic liquid include an organic solvent immiscible with water and a mixed solution of two or more of such organic solvents. The organic solvent immiscible with water typically includes, but is not limited to, solvents referred to as nonpolar solvents.

The non-hydrophilic liquid includes, but is not particularly limited to, various organic solvents immiscible with water. Examples thereof include the biocompatible liquid disclosed in the present specification without limitation. Typically, the non-hydrophilic liquid includes various so-called oil and liquids having a fluorocarbon structure. The fluorocarbon structure refers to the structure having at least one —C—F structure in which a fluorine directly hinds to a carbon. The non-hydrophilic liquid includes, but is not limited to, liquids indicated in the Table hereinbelow.

The term “non-hydrophilic substance” as used in the present specification means, in addition to the non-hydrophilic liquid described above, a substance that in itself has poor solubility or dispersibility in a hydrophilic liquid. A substance may be referred to as a non-hydrophilic substance when the substance is not dissolved or uniformly dispersed in water at 0° C. or higher anti 50° C. or lower. The non-hydrophilic substance may be in a liquid, solid or gas state under the condition at which the substance is not dissolved or dispersed in water. ‘The non’-hydrophilic substance may be organic, inorganic or complex thereof.

The terms “cell” as used in the present specification includes, without particular limitation, animals, plants and microbes as well as viruses. Animal cells include, but are not limited to, mammal cells including human cells and non-mammal animal cells. Plant cells that may be used include, without particular limitation, various plant cells. Microbial cells include, without particular limitation, various microbial (prokaryotic and eukaryotic) cells.

Examples of the cells that may be preferably used include animal cells derived from humans such as human airway epithelial cells, alveolar epithelial cells, intestinal epithelial cells, keratinocytes, corneal epithelial cells, fibroblasts, vascular endothelial cells, osteoblasts, mesenchymal stem cells, ES cells and iPS cells.

The cells may be or may not be adherent cells. The activity or growth property of adherent cells may be ensured by using a support that serves as an appropriate scaffold. It is also preferable to use a support for retaining cells when non-adherent cells are used.

The cells may be bound to each other and/or bound to the extracellular matrix. Namely, the cells may be a structure having a desired three-dimensional shape such as a sheet, a tube or a laminate. The cells may be biological tissues or organs or parts thereof. Further, the cells may be a structure derived from stem cells constituted according to stem cell engineering.

The term “cell” in the context of “cytotoxicity” and “cytocompatibility (or biocompatibility)” as used herein essentially means a naked cell devoid of an extracellular component. From this point of view, main causes of cytotoxicity may include (1) lysis of cell membranes by a liquid; (2) disruption of cell metabolism due to dispersion and infiltration of a liquid into cells resulting in reaction with and denaturation of biological components; and (3) damage on DNA.

Representative, non-limiting examples of the present invention will now be described in further detail with reference to the attached drawings. This detailed description is merely intended to teach a person of skill in the art further details for practicing preferred aspects of the present teachings and is not intended to limit the scope of the invention. Furthermore, each of the additional features and teachings disclosed below may be utilized separately or in conjunction with other features and teachings to provide improved biocompatible liquid, method for screening same, biocompatible liquid and method of evaluation of biocompatibility of liquid.

Moreover, combinations of features and steps disclosed in the following detail description may not be necessary to practice the invention in the broadest sense, and are instead taught merely to particularly describe representative examples of the invention. Furthermore, various features of the above-described and below-described representative examples, as well as the various independent and dependent claims, may be combined in ways that are not specifically and explicitly enumerated in order to provide additional useful embodiments of the present teachings.

All features disclosed in the description and/or the claims are intended to be disclosed separately and independently from each other for the purpose of original written disclosure, as well as for the purpose of restricting the claimed subject matter, independent of the compositions of the features in the embodiments and/or the claims. In addition, all value ranges or indications of groups of entities are intended to disclose every possible intermediate value or intermediate entity for the purpose of original written disclosure, as well as for the purpose of restricting the claimed subject matter.

(Hansen Solubility Parameter: HSP)

Hansen solubility parameter (HSP) as used herein is defined as described below.

HSP of a liquid is represented by a combination of 3 partitioned cohesive energy density values, i.e. δD: dispersion force, δP: polar force and δH: hydrogen bonding force, which are indicated by [J/cm³]^{1/2} or [MPa]^{1/2}. HSPs of liquids may be obtained as registered values or empirically calculated values in a commercial software, HSPiP 4th Edition, version 4.0.04.

The above software may be obtained from the website, for example, http://hansen-solubility.com/index.html. HSPs may be determined by using the software according to the report by Hansen et al. (e.g. C. M. Hansen solubility parameters: a user's handbook 2 edition, CRC press, 2007, ISBN-10: 0849372488).

The distance D between two HSPs in the Cartesian coordinate system of an HSP space may be calculated as follows:

D=[4×(δD₁−δD₂)²+(δP₁−δP₂)²+(δH₁−δH₂)²]^{1/2} [Math. 1]

When the D according to the above equation (Math 1) is decreased, two compounds have an increased interaction (solubility, swelling, etc.), while when the distance D is increased, the interaction is decreased. Thus, by defining, in the Cartesian coordinate system of an HSP space, a spherical space of the distance D having one HSP (δD, δP, δH) at the center, a certain property of a compound (liquid or solute such as polymer) may be characterized. When the distance D (radius) is decreased, the number of similar compounds (liquids) that interact is decreased, while when the distance D is increased, the number of similar compounds (liquids) that interact is increased.

An index for quantitatively evaluate the interaction between a certain liquid (δD₁, δP₁, δH₁) and a solute (δD₂, δP₂, δH₂) may be the relative energy difference (RED) represented by the following equation:

RED=D/R.

In the above equation, D is defined by the above formula (Math 1) and R is an interaction radius of solute. When RED is less than 1, a solute shows preferable solubility, swelling and the like into a liquid, while when RED is 1 or more, a solute shows unfavorable solubility and swelling into a liquid.

The HSP of a single solvent is expressed as the HSP of the single solvent and the HSP of a mixed solvent is expressed as a weighted average of HSPs of single solvents. The HSP of a mixed solvent is expressed as follows. In the equation, δDₘᵢₓ, δPₘᵢₓ and δHₘᵢₓ are a set of HSP of a mixed solvent, Cᵢ^{v} is a volume fraction of an i^{th} liquid and δDᵢ, δPᵢ and δHᵢ are a set of HSP of an i^{th} liquid.

$$δ {D}_{mix} = {\sum }_{i} {c}_{i}^{v} δ {D}_{i} , δ {P}_{mix} = {\sum }_{i} {c}_{i}^{v} δ {P}_{i} , δ {H}_{mix} = {\sum }_{i} {c}_{i}^{v} δ {H}_{i} \qquad [ Math . 2 ]$$

(Molar Volume)

In addition to HSP, the molar volume of a liquid may affect biocompatibility. This is because the liquid molar volume relates to intermolecular interactions and kinetic phenomena (diffusion, etc). When the molar volume is increased, a liquid tends to have a decreased solubility in cell membrane components constituting living organisms, a decreased infiltration or diffusion and thus an increased biocompatibility. On the other hand, when the molar volume is decreased, a liquid tends to have an increased infiltration or diffusion into cells and a decreased biocompatibility. The molar volume of a mixed solvent may be expressed as a weighted average of molar volumes of respective single solvents.

$${V}_{mix}^{m} = {\sum }_{i} {c}_{i}^{m} {V}_{i}^{m} \qquad [ Math . 3 ]$$

In the above equation, Vₘᵢₙ^{m} is a molar volume of a mixed solvent, Cᵢ^{m} is a molar fraction of an i^{th} solvent and Vᵢ^{m} is a molar volume of an i^{th} solvent. The molar volume of a liquid as used herein may be based on the registered value or empirically calculated value in the database of the HSPiP 4th Edition, version 4.0.04. The molar volume is indicated by cm³/mol or cc/mol.

(Biocompatibility)

Biocompatibility as used herein means compatibility to naked cells that are devoid of external components other than cell membranes separating the cells from the outside world, Naked cells mean living cells separated from the outside world only through cell membranes and examples thereof include cells that are devoid of extracellular components such as mucous membranes, horn, cell walls or outer membranes. Naked cells may be single cells or a collection thereof, or biological tissues or organs as far as they are naked cells.

The present disclosure is hereinafter specifically described by referring to the drawings.

(Biocompatible Liquid)

The biocompatible liquid according to the present disclosure may have a HSP that is compatible to a cell to which the liquid is applied. The compatible HSP can be determined on the basis of HSP threshold information associated with biocompatibility obtained from a plurality of liquids for which a level of biocompatibility to a cell to which the liquids are applied has been established in advance and HSPs of the liquids.

The biocompatible liquid according to the present disclosure may have a liquid molar volume that is compatible to a cell to which the liquid is applied. The compatible liquid molar volume can be determined on the basis of molar volume threshold information associated with biocompatibility obtained from a plurality of liquids for which a level of biocompatibility to a cell to which the liquids are applied has been established in advance and molar volumes of the liquids.

HSP threshold information is used in its own or used in combination with molar volume information. Molar volume threshold information is used in its own or used in combination with HSP threshold information.

### HSP Threshold Information

### First Embodiment

According to the present, disclosure, HSP threshold information may be defined based on HSP sphere defined by a predetermined core (δD, δP, δH) in a HSP space and a predetermined interaction radius R and the compatible HSP may be present within the HSP sphere. The phrase “within the HSP sphere” means a HSP identical to HSP coordinates that define the exterior edge of the HSP sphere or to HSPs existing inside of the HSPs defining the exterior edge of the HSP sphere.

The HSP sphere serving as HSP threshold information is determined in advance on the basis of HSPs of one or more liquids biocompatible to a cell to which the liquids are applied, HSP threshold information may be in some cases applied to a plurality of cells to which the liquid is applied. For example, HSP threshold information to be applied to a cultured cell that is widely used may be applied to other cultured cells and the like. On the other hand, HSP threshold information may vary according to a cell to which the liquid is applied. For example, HSP threshold information may be different for a human cell and a microbial cell such as yeast. HSP threshold information may be different between human cells from different sources. HSP threshold information may be determined from biocompatibility to an intended cell to which the liquid is applied obtained by carrying out a biocompatibility test of the liquid to the cell, and the HSP of the liquid.

(Molar Volume Threshold Information)

The HSP sphere serving as HSP threshold information may be defined in association with molar volume threshold information of a liquid. Biocompatibility to a cell to which a liquid is applied may vary according the molar volume of the molar volume of the liquid. As described above, when the molar volume is increased, biocompatibility to a cell to which a liquid is applied tends to be increased, while when the molar volume is decreased, biocompatibility to a cell to which a liquid is applied tends to be decreased.

Molar volume threshold information serving as an index of biocompatibility may vary according to a cell to which a liquid is applied. Molar volume threshold information may be determined from biocompatibility to an intended cell to which the liquid is applied obtained by carrying out a biocompatibility test of the liquid to the cell, and the molar volume of the liquid. The method for obtaining molar volume threshold information is specifically described hereinafter.

(Obtaining Threshold Information)

HSP threshold information and molar volume threshold information described above may be obtained, for example, according to the following manner. Thus, HSP threshold information may be obtained by evaluating biocompatibility of one or more test liquids to a naked cell to which the liquids are applied and associating intended biocompatibility and HSPs to determine a HSP core and an interaction radius that accommodate the intended biocompatibility. Molar volume threshold information may be obtained by associating intended biocompatibility and molar volumes to determine a threshold of the molar volume that accommodates the intended biocompatibility:

Biocompatibility of a test liquid may be evaluated according to the following manner. First, the liquid is directly brought into contact with a cell to be applied (e.g. a human cell including a human normal airway epithelial cell) for a certain period of time, e.g. 2 hours, followed by carrying out a known cytotoxicity test such as WST-8 assay. In the present disclosure, a naked cell, which is a cell to which a liquid is applied, is directly brought into contact with a test liquid, and thus threshold information for evaluating biocompatibility of the test liquid to a cell to which the test liquid is applied can be obtained in terms of essential significance.

Biocompatibility may be evaluated by using, for example, a normalized cell survival rate. The normalized cell survival rate is a numerical value obtained by dividing a survival rate of a test liquid by a survival rate of a liquid medium without cytotoxicity (0.5% FBS-containing RPMI-1640) after liquid toxicity tests with the survival rate of the liquid medium without cytotoxicity (0.5% FBS-containing RPMI-1640) being 1.0.

Next, a test liquid having a predetermined normalized cell survival rate or more, for example 0.7 or more, is defined as a biocompatible liquid having positive biocompatibility. The normalized cell survival rate may be appropriately established according to intended biocompatibility. Namely, when bioeompatibility is intended to be high, the survival rate may be 0.7 or more, more preferably 0.8 or more, still more preferably 0.9 or more, yet more preferably 0.95 or more. When biocompatibility is intended to be moderate, the survival rate may be in any range of, for example, 0.2 or more and 0.7 or less. When biocompatibility is intended to be low, the survival rate may be, for example, less than 0.2.

Meanwhile the HSP of a test liquid is obtained from the software described above. The software for calculating HSPs may be obtained from the website (http://hansen-solubility.com/index.html) described above or the like. The HSP may be determined on the basis of the software according to the report by Hansen et al. described above.

Biocompatibility indexes such as normalized cell survival rates and HSPs are obtained preferably for a plurality of, more preferably 5 or more, still more preferably 7 or more, yet more preferably 10 or more liquids. From the normalized cell survival rates and the like and HSPs of the test liquids, the core (δD, δP, δH) of the HSPs is determined by the Hansen sphere method as an index of the intended normalized cell survival rate, i.e. intended biocompatibility, and the core serves a core of the HSP space of the intended biocompatible liquid. The interaction radius, in addition to the core, may be obtained by using the software according to the calculation method described in the report by Hansen et al, described above.

In the similar manner as above, the molar volume of a test liquid is obtained. The molar volume may also be obtained by the software described above or by other well-known methods. Accordingly a threshold of the molar volume that serves as an index of intended normalized cell survival rate, i.e. positive biocompatibility, may be obtained from the intended normalized cell survival rate and the molar volume.

For example, the normalized cell survival rate tends to depend on the molar volume of a test liquid. The normalized cell survival rate tends to be different according to 3 regions of the molar volume of liquid. The 3 regions typically include strong toxicity, moderate/high toxicity and low toxicity. When molar volume threshold information is the molar volume at which the tendency of the normalized cell survival rate is changed, one of thresholds of the molar volume may be, for example, 125 cm³/mol and the other threshold may be 330 cm³/mol. In this case, there are the regions of molar volumes of (1) 125 cm³/mol or less, (2) more than 125 cm³/mol and less than 330 cm³/mol and (3) 330 cm³/mol or more. The regions (1) to (3) may be referred to as a strong cytotoxicity (low biocompatibility) region, a moderate toxicity/non-toxicity (moderate biocompatibility thigh biocompatibility) region and a non-cytotoxicity (high biocompatibility) region.

Cytotoxicity is believed to be exhibited by infiltration of a liquid into cells and dissolution of cell components in the liquid. Therefore, a liquid having a high molar volume such as the one in the region (3) is believed to have poor ability to infiltrate into cells or to dissolve other substances, and thus to have low cytotoxicity. On the other hand, a liquid having a low molar volume such as the on in the region (1) is believed to be liable to infiltrate into cells or to dissolve other substances, and thus to have strong toxicity. Cytotoxicity (biocompatibility) of a liquid in the region (2) may be determined by significantly depending on the HSP, which is the property of the liquid per se.

Accordingly when the molar volume is in the region (2) (molar volume: more than 125 cm³/mol and less than 330 cm³/mol), the normalized cell survival rate significantly varies according to the HSP of the liquid. When the molar volume is in the region (3) (molar volume: 330 cm³/mol or more), the normalized cell survival rate may be in general hardly decreased and does not significantly depend on the HSP. When the molar volume is in the region (1) (molar volume: 125 cm³/mol or less), the liquid is strongly cytotoxic and in general cannot be biocompatible. When the molar volume s at or less than is at or less than this value, no liquid state can be obtained (it becomes gaseous state) at the standard state (25° C., 1 atm) even when the HSP is biocompatible.

The thresholds of the molar volume regions (1) to (3) as used herein may vary according to the cell to which the liquid is applied.

As described above, the HSP core may be determined by evaluating the normalized cell survival rate regardless of the molar volume. Alternatively the HSP core may be determined by taking molar volume threshold information of a test liquid into account Particularly, for a test liquid having a molar volume of less than 330 cm³/mol or a test liquid having a molar volume of more than 125 cm³/mol and less than 330 cm³/mol, a valid HSP core may be obtained by evaluating the normalized cell survival rate. For a test liquid having a molar volume of 330 cm³/mol or more, valid HSP threshold information may be obtained by evaluating the normalized cell survival rate.

Next, an interaction radius R is determined from the HSP core. For this purpose, the HSP of a test liquid and the distance D ([J/cm³]^{1/2}) in a HSP space from the calculated HSP core are obtained by using the software described above according to the report by Hansen et al. The interaction radius may be obtained by plotting the distance and the normalized cell survival rate. Similarly in this case, the distance and the normalized cell survival rate may be plotted on the horizontal and vertical axes, respectively, by taking molar volume threshold information into account, for example, for a test liquid having a molar volume of less than 330 cm³/mol or more than 125 cm³/mol and less than 330 cm³/mol. In the molar volume ranges, HSP significantly affects the normalized cell survival rate, and thus when the interaction radius is determined, a highly accurate interaction radius R can be determined by limiting the range of the molar volume. In this plot, the interaction radius R ([J/cm³]^{1/2}) may be, for example, a distance D that allows a normalized cell survival rate (e.g. 0.7 or more, preferably 0.8 or more, more preferably 0.9 or more, still more preferably 0.95 or more, yet more preferably about 1.0, etc.) for positive biocompatibility.

For example, for a test liquid having a molar volume of 330 cm³/mol or more, a plot is similarly generated, the distance D that allows the normalized cell survival rate for positive biocompatibility is determined, the normalized cell survival rate of a test liquid having an interaction radius that is below the interaction radius of the liquid having the molar volume in the range is evaluated, and thus valid HSP threshold information may be obtained. In this range of the molar volume, a highly accurate interaction radius R may be obtained by similarly limiting the range of the molar volume.

Examples of identification of biocompatibility of a liquid utilizing an example of HSP threshold information and molar volume threshold information are described as follows.

When a liquid has a molar volume of 330 cm³/mol or more, HSP threshold information may be defined based on the following HSP sphere and compatible HSP may be a HSP within the HSP sphere.

HSP sphere 1:

Core (δD, δP δH): (12.73, 2.33, 3.46) ([J/cm³]^{1/2})

Interaction radius R: 9.0 ([J/cm³]^{1/2})

(Boiling Point and/or Melting Point)

In addition to HSP and molar volume, boiling point and/or melting point may be taken into account for biocompatibility of a liquid. Boiling point and melting point affect convenience and ease in liquid handling of a biocompatible liquid. The boiling point is preferably above 33° C. When the boiling point is less than 33° C., handling of the liquid is difficult in a normal working environment (about 10 to 30° C.) generally suitable for survival of organisms. The melting point is preferably less than 25° C. When the melting point is 25° C. or more, handling of the liquid is difficult in a normal working environment (about 10 to 30° C.).

Examples of liquids having the compatible HSPs include the following liquids. Examples thereof also include mixed solvents of the following liquids that have HSPs within the HSP sphere. The name, CAS number and chemical formula based on Simplified Molecular Input Line Entry Syntax (Smiles) of liquids are shown in the following Table

TABLE 3

| | | CAS No. | |
|---|---|---|---|
| No. | Solvent name | SMILES | SMILES |
| 1 | Dibutyl Sebacate | 109-43-3 | O═C(OCCCC)CCCCCCCCC(OCCCC)═O |
| 2 | Di-Isodecyl Phthalate | 26761-40-0 | O═C(OCCCCCCCC(C)C)C1═CC═CC═C1C(OCCCCCCCC(C)C)═O |
| 3 | Ditridecyl Phthalate | 119-06-2 | CCCCCCCCCCCCCOC(═O)C1═CC═CC═C1C(═O)OCCCCCCCCCCCCC |
| 4 | Isopropyl Palmitate | 142-91-6 | CCCCCCCCCCCCCCCC(OC(C)C)═O |
| 5 | Methyl Oleate | 112-62-9 | CCCCCCCC\C═C/CCCCCCCC(OC)═O |
| 6 | Triisononyl Trimethilate | 53894-23-8 | O═C(OCCCCCCC(C)C)C1═CC(C(OCCCCCCC(C)C)═O)═C(C(OCCCCCCC(C)C)═ |
| | | | O)C═C1 |
| 7 | Glycerol Trioleate | 122-32-7 | CCCCCCCC/C═C\CCCCCCCC(OCC(OC(CCCCCCC\C═C/CCCCCCCC)═O)COC |
| | | | (CCCCCCC/C═C\CCCCCCCC)═O)═O |
| 8 | Ethyl Oleate | 111-62-6 | CCCCCCCCC═CCCCCCCCC(═O)OCC |
| 9 | OS-30 (Decamethyltetrasiloxane) | 141-62-8 | C[Si](C)(C)O[Si](C)(C)O[Si](C)(C)O[Si](C)(C)C |
| 10 | Tri-n-Butyl Acetyl Citrate | 77-90-7 | CCCCOC(═O)CC(CC(═O)OCCCC)(C(═O)OCCCC)OC(═O)C |
| 11 | Di-(2-Ethylhexyl)Azelate | 103-24-2 | CCCCC(CC)COC(═O)CCCCCCCC(═O)OCC(CC)CCCC |
| 12 | Dioctyl Adipate | 123-79-5 | CCCCCCCCOC(═O)CCCCC(═O)OCCCCCCCC |
| 13 | Trioctyl Phosphate | 1606-54-8 | CCCCCCCCOP(═O)(OCCCCCCCC)OCCCCCCCC |
| 14 | Butyl Oleate | 142-77-8 | CCCCCCCC\C═C/CCCCCCCC(OCCCC)═O |
| 15 | Decamethylcyclopentasiloxane | 541-02-6 | [Si]1(C)(C)O[Si](C)(C)O[Si](C)(C)O[Si](C)(C)O[Si](O1)(C)C |
| 16 | Nonadecane | 629-92-5 | CCCCCCCCCCCCCCCCCCC |
| 17 | Bis (2-Ethylhexyl)Phthalate | 117-81-7 | O═C(OCC(CC)CCCC)C1═CC═CC═C1C(OCC(CC)CCCC)═O |
| 18 | Dodecamethylcyclohexasiloxane | 540-97-6 | O1[Si](O[Si](O[Si](O[Si](O[Si](O[Si]1(C)C)(C)C)(C)C)(C)C)(C)C)(C) C |
| 19 | Tetradecamethylhexasiloxane | 107-52-8 | C[Si](C)(C)O[Si](C)(C)O[Si](C)(C)O[Si](C)(C)O[Si](C)(C)O[Si](C)(C)C |
| 20 | Eicosamethylnonasiloxane | 2652-13-3 | C[Si](C)(C)O[Si](C)(C)O[Si](C)(C)O[Si](C)(C)O[Si](C)(C)O[Si](C)(C)O[Si](C)(C)O[Si] |
| | | | (C)(C)O[Si](C)(C)C |
| 21 | Squalane | 111-01-3 | C(C)(C)CCCC(C)CCCC(C)CCCCC(C)CCCC(C)CCCC(C)C |
| 22 | 1-Nonadecane | 18435-45-5 | C═CCCCCCCCCCCCCCCCCC |
| 23 | Eicosane | 3452-07-1 | CCCCCCCCCCCCCCCCCCC═C |
| 24 | Tetradecylcyclohexane | 1795-18-2 | CCCCCCCCCCCCCCC1CCCCC1 |
| 25 | Dihexyl Adipate | 110-33-8 | O═C(OCCCCCC)CCCCC(OCCCCCC)═O |
| 26 | Dinonyl Ether | 2456-27-1 | O(CCCCCCCCC)CCCCCCCCC |
| 27 | Diisooctyl Phthalate | 27554-26-3 | O═C(OCCCCCC(C)C)C1═CC═CC═C1C(OCCCCCC(C)C)═O |
| 28 | Hexadecyl Acetate | 629-70-9 | CCCCCCCCCCCCCCCCOC(C)═O |
| 29 | Octadecanoic Acid, Methyl Ester | 112-61-8 | CCCCCCCCCCCCCCCCCC(OC)═O |
| 30 | 2-Methyloctadecane | 1560-88-9 | CC(C)CCCCCCCCCCCCCCCC |
| 31 | 2-Methylnonadecane | 1560-88-7 | CC(C)CCCCCCCCCCCCCCCCC |
| 32 | Heneicosane | 629-94-7 | CCCCCCCCCCCCCCCCCCCCC |
| 33 | Docosane | 629-97-0 | CCCCCCCCCCCCCCCCCCCCCC |
| 34 | Tristearin | 555-43-1 | O═C(CCCCCCCCCCCCCCCCC)OCC(COC(CCCCCCCCCCCCCCCCC)═O)OC |
| | | | (CCCCCCCCCCCCCCCCC)═O |
| 35 | Trilaurin | 538-24-9 | CCCCCCCCCCCC(OCC(COC(CCCCCCCCCCC)═O)OC(CCCCCCCCCCC)═O)═O |
| 36 | Ethylhexadecanoate | 628-97-7 | O═C(OCC)CCCCCCCCCCCCCCC |
| 37 | 2,3-Dimethylheptadecane | 61866-03-9 | CC(C(CCCCCCCCCCCCCC)C)C |
| 38 | 3-Methyloctadecane | 6561-44-0 | CCCCCCCCCCCCCCCC(C)CC |
| 39 | 3-Methylnonadecane | 6418-45-7 | CCCCCCCCCCCCCCCCC(C)CC |

When a liquid has a molar volume of, for example, more than 125 cm³/mol and less than 330 cm³/mol, HSP threshold information may be the following HSP sphere and compatible HSP may be a HSP within the HSP sphere.

HSP sphere 2:

Core (δD, δP, δH): (12.73, 2.33, 3.46) ([J/cm³]^{1/2})

Interaction radius R: 3.4 ([J/cm³]^{1/2})

Examples of liquids having the compatible HSPs include the following liquids. Examples thereof also include mixed solvents of the following liquids that have HSPs within the HSP sphere.

TABLE 4A

| No. | Solvent name | CAS No. | SMILES |
|---|---|---|---|
| 40 | HCFC 225cb | 507-55-1 | ClC(F)(F)C(F)(F)C(F)([H])Cl |
| 41 | HFE 7000 | 375-03-1 | COC(F)(C(F)(C(F)(F)F)F)F |
| 42 | HFE 7100 | 153702-07-6 | COC(C(C(C(F)(F)F)(F)F)(F)F)(F)F |
| 43 | HFE 7500 | 297730-93-9 | CCOC(C(C(C(F)(F)F)(F)F)(F)F)(C(C(F)(F)F)(C(F)(F)F)F)F |
| 44 | HFE 8200 | NA | |
| 45 | Octamethylcyclotetrasiloxane | 556-87-2 | C[Si]1(C)O[Si](C)(C)O[Si](C)(C)O[Si](C)(C)O1 |
| 46 | 2,2,3,3,3-Pentafluoropropyl Methyl Ether | 378-16-5 | COCC(F)(F)C(F)(F)F |
| 47 | 1,1,2,3,3,3-Hexafluoropropyl Methyl Ether | 382-34-3 | COC(F)(C(C(F)(F)F)F)F |
| 48 | Difluoromethyl 2,2,3,3,3-Pentafluoropropyl Ether | 56880-81-2 | FC(OCC(C(F)(F)F)(F)F)F |
| 49 | 1-Trifluoromethyl-2,2,2-Trifluoroethyl Methyl Ether | 13171-18-1 | COC(C(F)(F)F)C(F)(F)F |
| 50 | 1,1,1,3,3,3-Hexafluoro-2-(Difluoromethoxy)Propane | 28103-08-2 | FC(F)(C(C(F)(F)F)OC(F)F)F |
| 51 | 1,1,2,2,2-Pentafluoroethyl Ethyl Ether | 22052-81-8 | CCOC(F)(C(F)(F)F)F |
| 52 | Pentafluoroethyl 2,2-Difluoroethyl Ether | 171182-85-9 | FC(OCC(F)F)(C(F)(F)F)F |
| 53 | Propane, 1,1,1,2,2-Pentafluoro-3-(1,1,2,2- | 50807-74-4 | FC(COC(F)(C(F)F)F)(C(F)(F)F)F |
| | Tetrafluoroethoxy)- | | |
| 54 | Propane, 2-(Difluoromethoxymethyl)-1,1,1,3,3,3- | 382-26-3 | COC(C(C(F)(F)F)C(F)(F)F)(F)F |
| | Hexafluoro- | | |
| 55 | 2,2,3,4,4,4-Hexafluorobutyl Difluoromethyl Ether | 69948-45-5 | FC(OCC(C(C(F)(F)F)F)(F)F)F |
| 56 | Butane, 1,1,1,2,3,3-Hexafluoro-4-(Trifluoromethoxy)- | 69948-43-2 | FC(COC(F)(F)F)(C(C(F)(F)F)F)F |
| 57 | Butane, 1-Ethoxy-1,1,2,2,3,3,4,4,4-Nonafluoro- | 183702-05-4 | CCOC(C(C(C(F)(F)F)(F)F)(F)F)(F)F |
| 58 | Propane, 1,1,1,3,3,3-Hexafluoro-2,2-Dimethoxy- | 754-50-7 | FC(C(OC)(OC)C(F)(F)F)(F)F |
| 59 | Butanoic Acid, Heptafluoro-, Methyl Ester | 356-24-1 | FC(F)(F)C(F)(F)C(F)(F)C(═O)OC |
| 60 | Propanoic Acid, Pentafluoro-, Ethyl Ester | 426-65-3 | CCOC(C(C(F)(F)F)(F)F)═O |
| 61 | Butanoic Acid, Heptafluoro-, Ethyl Ester | 356-27-4 | FC(F)(F)C(F)(F)C(F)(F)C(═O)OCC |
| 62 | 2-Butanone, 3,4,4,4-Tetrafluoro-3-(Trifluoromethyl)- | 80553-01-1 | CC(C(C(F)(F)F)(C(F)(F)F)F)═O |
| 63 | 3-Pentanone, 1,1,1,2,2,4,5,5,5-Nonafluoro-4- | 756-13-8 | O═C(C(C(F)(F)F)(C(F)(F)F)F)C(F)(C(F)(F)F)F |
| | (Trifluoromethyl)- | | |
| 64 | 3-Pentanone, 1,1,1,2,2,5,5,5-Octafluoro-4- | 61637-91-0 | O═C(C(C(F)(F)F)C(F)(F)F)C(F)(C(F)(F)F)F |
| | (Trifluoromethyl)- | | |
| 65 | 2-Hexanone, 3,3,4,4,5,5,6,6,6-Nonafluoro- | 678-18-2 | FC(F)(F)C(F)(F)C(F)(F)C(F)(F)C(C)═O |
| 66 | 3-Hexanone, 4,4,5,5,6,6,6-Heptafluoro- | 358-23-0 | O═C(C(C(C(F)(F)F)(F)F)(F)F)CC |
| 67 | 1,1,1,3,3,3-Hexafluoro Isopropyl Acrylate | 2160-89-6 | FC(F)(F)C(OC(═O)C═C)C(F)(F)F |
| 68 | 1,1,1,3,3,3-Hexafluoroisopropyl Methacrylate | 3063-94-3 | FC(F)(F)C(OC(═O)C(═C)C)C(F)(F)F |
| 69 | Ethyl Perfluorooctanoate | 3108-24-5 | FC(F)(F)C(F)(F)C(F)(F)C(F)(F)C(F)(F)C(F)(F)C(F)(F)C(═O)OCC |
| 70 | 1H,1H-Heptafluoro-n-Butyl Methacrylate | 13695-31-3 | FC(F)(F)C(F)(F)C(F)(F)COC(═O)C(═C)C |
| 71 | 1H,1H-Heptafluorobutyl Acrylate 424-84-6 | 424-84-6 | O═C(C([H])═C([H])/[H])OCC(F)(C(F)(C(F)(F)F)F)F |
| 72 | Heptafluoropropyl-1,2,2,2-Tetrafluoroethyl Ether | 3330-15-2 | FC(OC(F)(F)C(F)(F)C(F)(F)F)C(F)(F)F |
| 73 | 2-Pyran, 2,2,3,3,4,4,5,5,6,6-Decafluorotetrahydro- | 355-79-3 | FC(C(C(OC(C(F)1F)(F)F)(F)F)(F)F)1F |
| 74 | Furan, 2,2,3,3,4,4,5-Heptafluorotetrahydro-5- | 356-48-9 | FC(F)(OC1(F)C(F)(F)C(F)(F)F)C(F)(F)C1(F)F |
| | (Pentafluoroethyl)- | | |
| 75 | Furan, 2,2,3,4,5,5-Hexafluorotetrahydro-3,4- | 68088-53-9 | FC1(C(F)(F)F)C(F)(C(F)(F)F)C(F)(F)OC(F)1F |
| | Bis(Trifluoromethyl)- | | |
| 76 | Furan, 2,2,3,3,4,5,5-Heptafluorotetrahydro-4- | 61340-72-5 | FC1(F)C(F)(C(F)(C(F)(F)F)F)C(F)(F)OC(F)1F |
| | (Pentafluoroethyl)- | | |
| 77 | Furan, 2,2,3,3,4,5-Hexafluorotetrahydro-4,5- | 61340-71-4 | FC1(F)C(F)(C(F)(F)F)C(F)(C(F)(F)F)OC(F)1F |
| | Bis(Trifluoromethyl)- | | |
| 78 | Furan, 2,2,3,3,5,5-Hexafluorotetrahydro-4,4- | 61340-73-6 | FC1(F)C(C(F)(F)F)(C(F)(F)F)C(F)(F)OC(F)1F |
| | Bis(Trifluoromethyl)- | | |
| 79 | 2-Pyran, 2,2,3,3,4,4,5,6,6-Nonafluorotetrahydro-5- | 61340-74-7 | FC1(F)C(F)(F)C(F)(F)C(F)(C(F)(F)F)C(F)(F)O1 |
| 80 | Furan, 2,2,3,4,4,5-Hexafluorotetrahydro-3,5- | 67406-02-4 | F[C@](O1)(C(F)(F)F)C([C@](F)(C(F)(F)F)C1(F)F)(F)F |
| | Bis(Trifluoromethyl)-, | | |
| 81 | 2-Pyran, 2,2,3,3,4,4,5,6,6-Nonafluorotetrahydro-6- | 356-47-8 | C1(C(C(CC(C1(F)F)(F)F)(C(F)(F)F)F)(F)F)(F)F |
| 82 | Furan, 2,3,3,4,4,5-Hexafluorotetrahydro-2,5- | 59883-83-1 | FC1(C(F)(F)F)OC(F)(C(F)(F)F)C(F)(F)C(F)1F |
| | Bis(Trifluoromethyl)- | | |
| 83 | Oxepane Dodecafluoro | 788-41-0 | FC1(C(C(F)(F)OC(F)(F)C(F)(F)C1(F)F)(F)F)F |
| 84 | Trimethyl(2-Heptafluoropropoxy-1,1,2- | f341 | C[Si](C)(C(F)(C(OC(F)(C(F)(C(F)(F)F)F)F)F)F)C |
| | Trifluoroethyl)Silane | | |
| 85 | Nonafluoro-Cyclopentane | 378-66-8 | FC(C(F)(C(F)(C(F)1F)F)F)C1(F)F |
| 86 | 1,1,1,2,2,3,3-Heptafluoro-Pentane | 754-68-7 | CCC(C(C(F)(F)F)(F)F)(F)F |
| 87 | Difluoromethyl 1,1,2,3,3,3-Hexafluoropropyl Ether | 58860-85-6 | FC(F)(C(C(OC(F)F)(F)F)F)F |
| 88 | Perfluor-Tert-Buthylamine | 2809-02-9 | FC(F)(F)C(N)(C(F)(F)F)C(F)(F)F |
| 89 | Decafluoro-1-Trifluoromethyl-Piperidine | 359-71-7 | C1(C(C(N(C(C1(F)F)(F)F)C(F)(F)F)(F)F)(F)F)(F)F |
| 90 | 1,1,2,2,2-Pentafluoroethyl 1,1,2-Trifluoroethyl Ether | f781 | FC(OC(F)(C(F)(F)F)F)(CF)F |

TABLE 4B

| No. | Solvent name | CAS No. | SMILES |
|---|---|---|---|
| 91 | Ethyl 1,1,2,2,3,3,3-Heptafluoropropyl Ether | 22052-88-4 | CCOC(C(C(F)(F)F)(F)F)(F)F |
| 92 | Propane, 1-(2,2-Difluoroethoxy)-1,1,2,2,3,3,3- | 176310-28-4 | FC(F)(C(F)(C(OCC(F)F)(F)F)F)F |
| | Heptafluoro- | | |
| 93 | 2,2,2-Trifluoroethyl 1,1,2,2,3,3,3- | 142459-08-7 | FC(F)(C(F)(C(OCC(F)(F)F)(F)F)F)F |
| | Heptafluoropropyl Ether | | |
| 94 | 1,1,2,2,2-Pentafluoroethyl 2,2,3,3- | f788 | FC(OCC(F)(F)C(F)F)(C(F)(F)F)F |
| | Tetrafluoropropyl Ether | | |
| 95 | 1,1,2,2,2-Pentafluoroethyl 2,2,3,3,3- | 165653-44-4 | FC(C(COC(C(F)(F)F)(F)F)(F)F)(F)F |
| | Pentafluoropropyl Ether | | |
| 96 | 2,2,3,4,4,4-Hexafluorobutyl Methyl Ether | 58705-93-4 | COCC(F)(C(C(F)(F)F)F)F |
| 97 | 2,2,3,3,4,4,4-Heptafluorobutyl Methyl Ether | 376-98-7 | COCC(F)(C(F)(C(F)(F)F)F)F |
| 98 | 1,1,2,2,3,3,3-Heptafluoropropyl 2,2,3,3- | f790 | FC(F)(C(F)F)COC(C(F)(C(F)(F)F)F)(F)F |
| | Tetrafluoropropyl Ether | | |
| 99 | 1,1,2,2,3,3,3-Heptafluoropropyl 2,2,3,3,3- | f791 | FC(OCC(F)(C(F)(F)F)F)(C(F)(C(F)(F)F)F)F |
| | Pentafluoropropyl Ether | | |
| 100 | 1-Hexane, 3,3,4,4,5,5,6,6,6-Nonafluoro- | 19430-93-4 | FC(F)(F)C(F)(F)C(F)(F)C(F)(F)C═C |
| 101 | 2H-Cyclopenta[B]Furan, | 72925-02-1 | FC1(F)C(F)(F)OC2(F)C(F)1C(F)(F)C(F)(F)C(F)2F |
| | Dodecafluorohexahydro- | | |
| 102 | 2H-Pyran, 2,2,3,3,4,4,5,6,6- | 67405-98-6 | FC1(F)OC(F)(F)C(F)(C(F)C(F)(F)F)F)C(F)(F)C(F)1F |
| | Nonafluorotetrahydro-5- | | |
| 103 | Furan, 2,2,3,3,4,5,5-Heptafluoro-4- | 67405-98-5 | FC1(C(F)(C(C(F)(F)F)(F)F)F)C(F)(F)C(F)(F)OC(F)1F |
| | (Heptafluoropropyl)Tetrahydro- | | |
| 104 | 2H-Pyran, 2,2,3,4,4,5,5,6- | 65083-03-8 | FC1(F)OC(F)(C(F)(F)F)C(F)(F)C(F)(F)C(C(F)(F)F)1F |
| | Octafluorotetrahydro-3,6- | | |
| 105 | Furan, 2,2,3,4,4,5-Hexafluorotetrahydro-3- | 57405-94-1 | F[C@]1(C(F)(C(F)(F)F)F)C([C@@](F)(C(F)(F)F)OC(F)1F(F)F |
| | (Pentafluoroethyl)-5- | | |
| | (Trifluoromethyl)-, Cis- | | |
| 106 | Furan, 2,2,3,4,4,5-Hexafluorotetrahydro-3- | 67422-92-8 | F[C@@]1(C(F)(C(F)(F)F)F)C([C@@](F)(C(F)(F)F)OC(F)1F)(F)F |
| | (Pentafluoroethyl)-5- | | |
| | (Trifluoromethyl)-, Trans- | | |
| 107 | Furan, 2,2,3,4,4,5-Hexafluorotetrahydro-5- | 68063-14-9 | F[C@@]1(C(F)(F)F)C([C@@](F)(C(F)(C(F)(F)F)F)OC(F)1F)(F)F |
| | (Pentafluoroethyl)-3- | | |
| | (Trifluoromethyl)-, Trans- | | |
| 108 | Furan, 2,2,3,4,4,5-Hexafluorotetrahydro-5- | 68063-02-5 | F[C@]1(C(F)(F)F)C([C@@](F)(C(F)(C(F)(F)F)F)OC(F)1F)(F)F |
| | (Pentafluoroethyl)-3- | | |
| | (Trifluoromethyl)-, Cis- | | |
| 109 | Furan, 2,3,3,4,4,5-Hexafluorotetrahydro-2- | 74942-11-3 | FC1(F)C(F)(F)C(F)(C(F)(C(F)(F)F)F)OC(C(F)(F)F)1F |
| | (Pentafluoroethyl)-5- | | |
| | (Trifluoromethyl)- | | |
| 110 | 2H-Pyran, 2,2,3,3,4,4,5,5,6- | 377-81-1 | FC1(F)C(F)(F)C(F)(F)C(F)(F)C(F)(C(F)(C(F)(F)F)F)O1 |
| | Nonafluorotetrahydro-6- | | |
| 111 | Benzofuran, Tetradecafluorooctahydro- | 55751-36-5 | FC1(C(F)(F)C(F)(F)C(F)(F)C(F)2F)C2(F)OC(F)(F)C(F)1F |
| 112 | 2H-Cyclopenta[B]Furan, | 74403-40-0 | FC1(F)C(F)(C(F)(F)F)C(C(F)(F)C(F)(F)C(F)2F)(F)C2(F)O1 |
| | 2,2,3,3A,4,4,5,5,6,6,6A- | | |
| | Undecafluorohexahydro-3-(Trifluoromethyl)- | | |
| 113 | Furan, 2,2,3,3,4,4,5-Heptafluorotetrahydro- | 335-36-4 | FC(F)(OC1(F)C(F)(F)C(F)(F)C(F)(F)C(F)(F)F)C(F)(F)C1(F)F |
| | 5-(Nonafluorobutyl)- | | |
| 114 | 2H-Pyran, 2,2,3,3,4,4,5,5,6-Nonafluoro-6- | 335-35-3 | FC(F)(OC(C1(F)F)(F)C(F)(F)C(F)(F)C(F)(F)F)C(F)(F)C1(F)F |
| 115 | Furan, 2,2,3,4,4,5-Hexafluorotetrahydro-3,5- | 68063-10-5 | FC1(C(F)(C(F)(F)F)F)C(F)(F)C(F)(C(F)(C(F)(F)F)F)C(F)(F)O1 |
| | Bis(Pentafluoroethyl)- | | |
| 116 | Furan, 2,2,3,3,4,5,5-Heptafluorotetrahydro- | 646-85-5 | FC1(F)C(F)(F)C(F)(C(C(F)(C(C(F)(F)F)(F)F)F)(F)F)C(F)(F)O1 |
| | 4-(Nonafluorobutyl)- | | |
| 117 | 2H-Pyran, 2,2,3,3,4,4,5,6,6-Nonafluoro-5- | 801-26-3 | FC1(F)C(F)(F)C(F)(F)C(F)(C(F)(C(C(F)(F)F)(F)F)F)C(F)(F)O1 |
| 118 | Furan, 2,2,3,4,4,5-Hexafluoro-3- | 68063-05-1 | FC1(C(F)(F)F)C(F)(F)C(F)(C(F)(C(F)C(F)(F)F)F)F)C(F)(F)O1 |
| | (Heptafluoropropyl)Tetrahydro-5- | | |
| | (Trifluoromethyl)- | | |
| 119 | Furan, 2,2,3,4,4,5-Hexafluoro-5- | 68063-15-0 | F[C@](OC([C@](F)1C(F)(F)F)(F)F)(C(F)(C(F)(C(F)(F)F)F)F)C1(F)F |
| | (Heptafluoropropyl)Tetrahydro-3- | | |
| | (Trifluoromethyl)-, Trans- | | |
| 120 | Furan, 2,2,3,4,4,5-Hexafluoro-5- | 68063-05-8 | F[C@](OC([C@@](F)1C(F)(F)F)(F)F)(C(F)(C(F)(C(F)(F)F)F)F)C1(F)F |
| | (Heptafluoropropyl)Tetrahydro-3- | | |
| | (Trifluoromethyl)-, Cis- | | |
| 121 | Furan, 2,2,3,4,4,5-Hexafluoro-2- | 74942-12-4 | FC1(C(F)(C(F)(C(F)(F)F)F)F)C(F)(F)C(F)(F)C(F)(C(F)(F)F)O1 |
| | (Heptafluoropropyl)Tetrahydro-5- | | |
| | (Trifluoromethyl)- | | |
| 122 | Furan, Tetrahydro-2-[2,2,2-Trifluoro-1,1- | 73416-03-2 | FC(C(C(F)(F)F)(C(F)(F)F)C1CCCO1)(F)F |
| | Bis(Trifluoromethyl)Ethyl]- | | |
| 123 | Butane, 1,1,1,2,2,3,3,4,4-Nonafluoro-4- | 559-29-5 | FC(OC(C(C(C(F)(F)F)(F)F)(F)F)(F)F)(F)F |
| | (Trifluoromethoxy)- | | |
| 124 | Propane, 1,1,1,2,3,3-Hexafluoro-3-(2,2,2- | 883-95-3 | FC(COC(C(C(F)(F)F)F)(F)F)(F)F |
| | Trifluoroethoxy)- | | |
| 125 | Propane, 1,1,1,3,3,3-Hexafluoro-2-Methoxy- | 68670-22-2 | COC(C(F)(F)F)(C(F)(F)F)C(F)(F)F |
| | 2-(Trifluoromethyl)- | | |
| 126 | Butane, 1,1,1,2,3,4,4,4-Octafluoro-2- | 42551-02-0 | COC(C(C(F)(F)F)F)(C(F)(F)F)F |
| | Methoxy | | |
| 127 | Propane, 1,1,1-Trifluoro-2-(1,1,2,2- | 50807-72-2 | [H]C(OC(C(F)F)(F)F)(C)C(F)(F)F |
| | Tetrafluoroethoxy)- | | |
| 128 | Propane, 1-Ethoxy-1,1,2,3,3,3-Hexafluoro- | 380-34-7 | CCOC(F)(C(C(F)(F)F)F)F |
| 129 | Butane, 1,1,1,2,2,3,3,4,4-Nonafluoro-4- | 71646-78-6 | FC(OC(C(F)(C(C(F)(F)F)(F)F)F)(F)F)(C(F)(F)F)F |
| | (Pentafluoroethoxy)- | | |

TABLE 5A

| No. | Solvent name | CAS No. | SMILES |
|---|---|---|---|
| 130 | Propane, 1,1′-Oxybis[1,1,2,2,3,3,3-Heptafluoro- | 356-52-7 | FC(F)(OC(F)(F)C(F)(F)C(F)(F)F)C(F)(F)C(F)(F)F |
| 131 | Ethane, 1,1,2,2-Tetrafluoro-1,2- | 356-70-7 | FC(OC(F)(C(OC(F)(C(F)(F)F)F)(F)F)F)(C(F)(F)F)F |
| | Bis(Pentafluoroethoxy)- | | |
| 132 | Propane, 1,1,1,2,3,3-Hexafluoro-3- | 1000-28-8 | FC(F)(C(C(OCC(F)(C(F)(F)F)F)(F)F)F)F |
| | (2,2,3,3,3-Pentafluoropropoxy)- | | |
| 133 | Propane, 1,1′-Oxybis[1,1,3,3,3-Pentafluoro- | 66711-94-2 | FC(OC(CC(F)(F)F)(F)F)(CC(F)(F)F)F |
| 134 | Propane, 1,1,1,2,3,3-Hexafluoro-3- | 65064-78-0 | FC(OCC(F)(C(F)F)F)(C(C(F)(F)F)F)F |
| | (2,2,3,3-Tetrafluoropropoxy)- | | |
| 135 | Propane, 2-(Ethoxydifluoromethyl)-1,1,1,3,3,3- | 380-30-3 | [H]C(C(OCC)(F)F)(C(F)(F)F)C(F)(F)F |
| | Hexafluoro- | | |
| 136 | Butane, 2-Ethoxy-1,1,1,2,4,4,4-Heptafluoro- | 106693-05-4 | FC(OCC)(C(F)(F)F)CC(F)(F)F |
| 137 | Propane, 1,1,1,2,3,3-Hexafluoro-3-Propoxy- | 357-99-3 | CCCOC(F)(C(C(F)(F)F)F)F |
| 138 | Propane, 1,1,1,2,3,3-Hexafluoro-3-(1-Methylethoxy)- | 357-97-1 | CC(C)OC(F)(C(C(F)(F)F)F)F |
| 139 | Propane, 1,1′-Oxybis[3,3,3-Trifluoro- | 674-65-7 | FC(CCOCCC(F)(F)F)(F)F |
| 140 | Propane, 2-Ethoxy-1,1,1,3,3,3-Hexafluoro-2-Methoxy- | 682-30-8 | COC(OCC)(C(F)(F)F)C(F)(F)F |
| 141 | Propane, 2-Methyl-2-(1,1,2,2-Tetrafluoroethoxy)- | 659-98-3 | CC(OC(F)(C(F)F)F)(C)C |
| 142 | Butane, 1,1,1,3,3-Pentafluoro-4-Methoxy-2- | f1528 | COCC(C(C(F)(F)F)C(F)(F)F)(F)F |
| | (Trifluoromethyl)- | | |
| 143 | Octafluoro-n-Difluoro-n-Difluoromethyl- | 67212-89-9 | FC(OC1(F)F)(C(N(C(F)F)C(F)1F)(F)F)F |
| | Morpholine(S) | | |
| 144 | Octafluoro-4-Trifluoromethyl-Morpholine | 382-28-5 | FC(N(C(F)(F)F)C(F)(C(OC(F)1F)(F)F)F)1F |
| 145 | Hexafluoro-3-Pentafluoroethyl-Oxazolidine | 432-10-0 | FC(N(C(F)(C(F)(F)F)F)C1(F)F)(OC(F)1F)F |
| 146 | 2,2,3,3,4,4,4-Heptafluoro-Butylamine | 374-99-2 | NCC(F)(C(F)(C(F)(F)F)F)F |
| 147 | Methyl-(2,2,3,3,3-Pentafluoro-Propyl)-Amine | 425-73-0 | CNCC(F)(F)C(F)(F)F |
| 148 | Ethanamine, 2,2-Difluoro-N,N-Bis(Trifluoromethyl)- | 176674-31-0 | FC(N(CC(F)F)C(F)(F)F)(F)F |
| 149 | Fluoromethyl 1,1,2,2,3,3,3-Heptafluoropropyl Ether | 184899-81-8 | FC(OCF)(C(F)(C(F)(F)F)F)F |
| 150 | 1,1,1,2,2,3,3,4,4-Nonafluorohexane | 38436-17-8 | CCC(C(F)(C(F)(C(F)(F)F)F)F)(F)F |
| 151 | n-C4f9oc3h7 | 72372-80-6 | FC(OCCC)(C(C(F)(C(F)(F)F)F)(F)F)F |
| 152 | n-C5f11och3 | 181214-74-4 | COC(C(C(F)(C(C(F)(F)F)(F)F)F)(F)F)(F)F |
| 153 | n-C5f11oc2h5 | 181214-75-5 | CCOC(C(C(F)(C(C(F)(F)F)(F)F)F)(F)F)(F)F |
| 154 | Cf3cf(Ch2ch3)Ocf3 | f2329 | FC(F)(C(OC(F)(F)F)(CC)F)F |
| 155 | Cf3cf2ocf(Cf3)Cf2och3 | 202464-47-9 | COC(F)(C(OC(F)(C(F)(F)F)F)(C(F)(F)F)F)F |
| 156 | (Cf3)2Chcocf2cf3 | f2334 | O═C(C(F)(C(F)(F)F)F)C(C(F)(F)F)C(F)(F)F |
| 157 | n-C3f7coch2ch3 | f2336 | CCC(C(F)(C(C(F)(F)F)(F)F)F)═O |
| 158 | (CF3)2CFCOCH3 | f2337 | O═C(C)C(C(F)(F)F)(C(F)(F)F)F |
| 159 | C5hf10no | f2339 | FC(N(C(F)1F)C(C(OC1(F)F)(F)F)(F)F)F |
| 160 | Methyl-<1,3,3,3-Tetrafluoro-2- | 360-53-2 | CO/C(F)═C(C(F)(F)F)/C(F)(F)F |
| | Trifluoromethyl-Propanyl>-Ether | | |
| 161 | Perfluoromethylcyclopentane | 1805-22-7 | C(F)(F)(F)C1(F)C(F)(F)C(F)(F)C(F)(F)C1(F)(F) |
| 162 | 1,2-Dichloro-1,1,2,3,3,3- | 661-97-2 | FC(F)(F)C(Cl)(F)C(F)(Cl)F |
| | Hexafluoropropane(F-2168a) | | |
| 163 | 1-Bromoperfluoroheptane | 375-88-2 | BrC(F)(F)C(F)(F)C(F)(F)C(F)(F)C(F)(F)C(F)(F)C(F)(F)F |
| 164 | 1-Bromoperfluorononane | 558-88-3 | FC(F)(F)C(F)(F)C(F)(F)C(F)(F)C(F)(F)C(F)(F)C(F)(F)C(F)(F)C(F)(Br)F |
| 165 | 1-Bromoperfluorooctane | 423-55-2 | FC(F)(F)C(F)(F)C(F)(F)C(F)(F)C(F)(F)C(F)(F)C(F)(F)C(F)(F)Br |
| 166 | 1,8-Dibromoperfluorooctane | 812-58-8 | BrC(F)(F)C(F)(F)C(F)(F)C(F)(F)C(F)(F)C(F)(F)C(F)(F)C(F)(F)Br |
| 167 | Dodecafluorodimethylcyclobutane | 28877-00-1 | FC1(F)C(C(F)(F)F)(F)C(F)(F)C1(F)C(F)(F)F |
| 168 | 3,3,4,4,5,5,5-Heptafluoro-1-Pentane | 71164-40-4 | C═CC(F)(C(F)(C(F)(F)F)F)F |
| 169 | 2-Iodononafluorobutane | 375-51-9 | FC(F)(F)C(F)(F)C(I)(F)C(F)(F)F |
| 170 | 1H,1H,2H-Perfluoro-1-Decane | 21652-58-4 | C═CC(F)(F)C(F)(F)C(F)(F)C(F)(F)C(F)(F)C(F)(F)C(F)(F)C(F)(F)F |
| 171 | Perfluoro-1,3-Dimethylcyclohexane | 335-27-3 | FC(F)(C(C(F)(C(F)(C1(F)F)C(F)(F)F)F)(C(F)(C1(F)F)F)F)F |
| 172 | 1H-Perfluoroheptane | 27213-81-2 | [H]C(F)(F)C(F)(F)C(F)(F)C(F)(F)C(F)(F)C(F)(F)C(F)(F)F |
| 173 | 5H-Perfluorohexane | 355-37-3 | FC(F)(F)C(F)(F)C(F)(F)C(F)(F)C(F)(F)C(F)(F)[H] |
| 174 | 1H,1H-Perfluoro-1-Octanol | 307-30-2 | FC(F)(F)C(F)(F)C(F)(F)C(F)(F)C(F)(F)C(F)(F)C(F)(F)CO |
| 175 | 1H,1H,2H-Perfluoro-1-Octane | 25291-17-2 | C═CC(F)(F)C(F)(F)C(F)(F)C(F)(F)C(F)(F)C(F)(F)F |
| 176 | Perfluoropiperidine | 836-77-1 | FC(C(F)(C(F)(C1(F)F)F)F)(C(F)(N1F)F)F |
| 177 | 1,1,1-Trifluoro-3-Methylbutane | 406-49-5 | FC(F)(CC(C)C)F |
| 178 | Perfluorooctyl Iodide | 507-83-1 | FC(C(F)(F)C(F)(F)C(F)(F)I)(F)C(F)(F)C(F)(F)C(F)(F)C(F)(F)F |
| 179 | 1,3-Dichlorohexafluoropropane (F-216) | 662-01-1 | FC(Cl)(F)C(F)(F)C(Cl)(F)F |
| 180 | Cf3cf(Ocf3)Ch2chf2 | f3000 | FC(CC(F)F)(C(F)(F)F)OC(F)(F)F |

TABLE 5B

| No. | Solvent name | CAS No. | SMILES |
|---|---|---|---|
| 181 | Cf3cf(Ocf3)Ch2cf3 | f3001 | FC(OC(CC(F)(F)F)(C(F)(F)F)F)(F)F |
| 182 | 1,4-Divinyloctafluorobutane | 678-65-9 | C═CC(C(F)(C(F)(C(C═C)(F)F)F)F)(F)F |
| 183 | 1,6-Divinyldodecafluorohexane | 1800-91-5 | FC(F)(C═C)C(F)(F)C(F)(F)C(F)(F)C(F)(F)C(F)(F)C═C |
| 184 | Perfluoroheptyl Iodide | 335-58-0 | IC(F)(F)C(F)(F)C(F)(F)C(F)(F)C(F)(F)C(F)(F)C(F)(F)F |
| 185 | Perfluoro-5-Methylhexyl Iodide | 3486-08-6 | IC(F)(F)C(F)(F)C(F)(F)C(F)(F)C(F)(C(F)(F)F)C(F)(F)F |
| 186 | Perfluoro-7-Methyloctyl Iodide | 865-77-0 | FC(F)(C(C(F)(F)F)(C(F)(C(F)(C(F)(C(F)(C(F)(C(F)(I)F)F)F)F)F)F)F)F |
| 187 | 2-Iodotetrafluoroethyl Heptafluoroisopropyl Ether | f4042 | FC(C(F)(F)F)(C(F)(F)F)OC(C(F)(I)F)(F)F |
| 188 | 1-(1,1-Difluoro-Ethoxy)-1,1,2,2,3,3,3- | f4055 | CC(OC(C(C(F)(F)F)(F)F)(F)F)(F)F |
| | Heptafluoropopane | | |
| 189 | Heptafuluoropropyl-1,2,2-Trifuluoroethyl Ether | f4056 | FC(C(OC(C(F)F)F)(F)F)(C(F)(F)F)F |
| 190 | 1,1,2,2,3,3,4,4-Octafluoro-5-Methoxypentane | f4057 | COCC(C(C(F)(C(F)F)F)(F)F)(F)F |
| 191 | Perfluoro(Isopentyl)Methylether | 203783-56-6 | COC(C(F)(C(C(F)(F)F)(C(F)(F)F)F)F)(F)F |
| 192 | Ethyl Tris(Trifluoromethyl)Methyl Ether | f4060 | CCOC(C(F)(F)F)(C(F)(F)F)C(F)(F)F |
| 193 | 2,2,4,4-Tetrafluoro-8-Trifluoromethyl-3- | (151504-21-8) | FC(C1C2C1C(OC(F)2F)(F)F)(F)F |
| | Oxabicyclo[3.1.0]Hexane | | |
| 194 | 2,2,4,4-Tetrafluoro-6,7-Bis(Trifluoromethyl)-3- | 160321-07-3 | FC(C1C(C(F)(F)F)C2C1C(OC(F)2F)(F)F)(F)F |
| | Oxabicyclo[3.2.0]Heptane | | |
| 195 | 1,1,1,2,2-Pentafluoro-3-(2,2,3,3,3-Pentafluoro- | 1422-73-7 | FC(F)(C(COCOCC(F)(C(F)(F)F)F)(F)F)F |
| | Propoxymethoxy)-Propane | | |
| 196 | 1,1,1,3,3,3-Hexafluoro-2-(2,2,2-Trifluoro-1- | 194039-81-1 | FC(C(OCOC(C(F)(F)F)C(F)(F)F)C(F)(F)F)(F)F |
| | Trifluoromethylethoxymethoxy)Propane | | |
| 197 | 1-(2,2,2-Trifluoroethoxy)-2-Trifluoromethoxy- | f4075 | FC(OCC(F)(F)F)(C(OC(F)(F)F)F)F |
| | 1,1,2-Trifluoroethane | | |
| 198 | 1-(2,2,3,3,3-Pentafluoropropoxy)-2-Trifluoromethoxy- | 226705-06-2 | FC(COC(C(OC(F)(F)F)F)(F)F)(C(F)(F)F)F |
| | 1,1,2-Trifluoroethane | | |
| 199 | 1,1,1,3,3,3-Hexafluoro-2-(1,1,2-Trifluoro-2- | 226705-05-1 | FC(OC(C(F)(F)F)C(F)(F)F)(C(OC(F)(F)F)F)F |
| | Trifluoromethoxyethoxy)Propane | | |
| 200 | Propane, 1,1,1,2,3,3-Hexafluoro-3-Methoxy-2- | 104159-55-9 | COC(C(OC(F)(F)F)(C(F)(F)F)F)(F)F |
| | (Trifluoromethoxy)- | | |
| 201 | 1-Ethoxy-1,1,2,3,3,3-Hexafluoro-2- | f4079 | CCOC(C(OC(F)(F)F)(C(F)(F)F)F)(F)F |
| | Trifluoromethoxypropane | | |
| 202 | 1,1,2,2-Tetrafluoro-1,2-Bis(2,2,2- | f4081 | FC(OCC(F)(F)F)(C(OCC(F)(F)F)(F)F)F |
| | Trifluoroethoxy)Ethane | | |
| 203 | 5-Trifluoromethyl-3,3,4,4,5,6,6,6-Octafluorohexane-2- | 244618-87-3 | O═C(C)C(C(C(C(F)(F)F)(C(F)(F)F)F)(F)F)(F)F |
| | On | | |
| 204 | 4,4,5,5,6,6,7,7,7-Nonafluoro-3-Heptanone | 296280-00-7 | O═C(CC)C(C(C(C(C)(F)(F)F)F)(F)F)(F)F |
| 205 | 4,4,5,5,6,6,7,7,8,8,8-Undecafluoro-3-Octanone | f4088 | CCC(C(C(C(C(F)(C(F)(F)F)F)(F)F)(F)F)(F)F)═O |
| 206 | 3,3,4,4,5,5,6,6,7,7,7-Undecafluoro-2-Heptanone | 2708-07-8 | O═C(C)C(C(F)(C(C(F)(C(F)(F)F)F)(F)F)F)(F)F |
| 207 | Methyl Perfluoro (Pyrrolidinemethyl) Ketone | f4090 | O═C(C)C(N(C(F)1F)C(C(C(F)1F)(F)F)(F)F)(F)F |
| 208 | Methylfluoro(2-(N,N-Diamino)Ethyl)Ketone | f4093 | O═C(C)C(C(N(C(F)(F)F)C(F)(F)F)(F)F)(F)F |
| 209 | 1,1-Difluoro-1-(2,2,3,3,5,5,6,6-Octafluoromorpholin- | f4094 | O═C(C)C(N(C(F)(C(OC(F)1F)(F)F)F)C1(F)F)(F)F |
| | 4-Yl)Acetone | | |
| 210 | 4-(Difluoromethyl)-2,6-Bis(Trifluoromethyl)- | 205876-74-0 | FC1(C(F)(F)F)OC(C(N(C(F)F)C(F)1F)(F)F)(C(F)(F)F)F |
| | 2,3,3,5,5,6-Hexafluoromorpholine | | |
| 211 | Ethyl 7H-Dodecafluoroheptanoate | 42287-85-4 | CCOC(C(F)(C(F)(C(F)(C(F)(C(F)(C(F)F)F)F)F)F)F)═O |
| 212 | Perfluorocyclohexane | 355-58-0 | FC(C(F)(C(F)(C1(F)F)F)F)(C(F)(C1(F)F)F)F |
| 213 | 1,3-Dichloro-1,1,3,3-Tetramethyldisiloxane | 2401-73-2 | C[Si](C)(O[Si](C)(CC)Cl)Cl |
| 214 | 1,5-Dichloro-1,1,3,3,5,5-Hexamethyltrisiloxane | 3582-71-8 | C[Si](C)(O[Si](C)(CC)Cl)O[Si](C)(C)Cl |
| 215 | Di-Tert-Butyl Ether | 6163-66-2 | CC(C)(C)OC(C)(C)C |
| 216 | Tetraethylsilane | 631-36-7 | CC[Si](CC)(CC)CC |
| 217 | Triethylsilane | 617-86-7 | CC[SiH](CC)CC |
| 218 | Chlorotrimethylsilane | 75-77-4 | Cl[Si](C)(C)C |
| 219 | Hexamethylcyclotrisiloxane | 541-05-9 | C[Si]1(O[Si](O[Si](O1)(C)C)(C)C)C |
| 220 | Hexamethyldisilazene | 999-97-3 | [Si](C)(C)(C)N[Si](C)(C)C |

When the liquid has a molar volume of 125 cm³/mol or less biocompatibility thereof may be negative.

### Second Embodiment

Alternatively, HSP threshold information may be defined based on a HSP sphere defined by a predetermined core (δD, δP, δH) in the HSP space ad a predetermined interaction radius and the compatible HSP may be present outside of the HSP sphere. The phrase “outside of the HSP sphere” means a HSP identical to HSP coordinates that define the exterior edge of the HSP sphere or to HSPs existing outside of the HSP sphere.

HSP threshold information of the HSP sphere to be excluded as described above is determined on the basis of HSP spheres of one or more cell components of a cell to which a liquid is applied. The HSP threshold information may vary according to a cell to which the liquid is applied, similarly to that described above. The cell component is not particularly limited. The cell component may be, for example, a universal component in the cell type including the cell to which the liquid is applied, or may be a characteristic cell component in the cell to which the liquid is applied. The cell component may be a cell membrane component or may be an intracellular organella or an intracellular substance.

Examples of the cell component include DNA, cholesterol, water, phosphatidyleholine, sphingomyelin, phosphatidylethanolamine and phosphatidylserine. The above-mentioned cell components may be divided into hydrophilic moieties and hydrophobic moieties to provide one or more, preferably 3 or more, more preferably 4 or more and still more preferably 5 or more HSP spheres selected from the group of 10 cell components. Yet more preferably, all of the above cell components may be used.

It is preferable that a liquid has a molar volume of 125 cm³/mol or more and a HSP is present outside of HSP spheres of one or more cell components selected from the group consisting of DNA, cholesterol, water, phosphatidylcholine (hydrophobic moieties 1, 2, hydrophilic moiety), phosphatidylethanolamine (hydrophobic moiety, hydrophilic moiety), sphingomyelin (hydrophilic moiety) and phosphatidylserine. HSPs (δD, δP, δH) and interaction radii of the cell components are shown below. The values indicated with italics with regard to HSPs are empirically calculated values or estimated values by the software described above. The inventors of the present invention established a defined value for the interaction radius R as 5 [J/cm³]^{1/2}.

TABLE 6

| ID | Cell constituent | SMILES | δD | δP | δH | R |
|---|---|---|---|---|---|---|
| C1 | Cholesterol | CC(C)CCCC(C)C1CCC2C1(CCC3C2CC═C4C3(CCC(C4)O)C)C | 20.4 | 2.8 | 9.4 | 12 |
| C2 | DNA | — | 19 | 30 | 11 | 11 |
| C3 | Cell membrane hydrophobic | CCCCCCCCCCCCCCCCC | 16 | 0 | 0 | 5 |
| | portion#1: Phosphatidylcholine | | | | | |
| | hydrophobic portion#1 | | | | | |
| C4 | Cell membrane hydrophobic | CCCCCCCC═CCCCCCCCC | 16 | 1.3 | 1.8 | 5 |
| | portion#2: Phosphatidylcholine | | | | | |
| | hydrophobic portion#2 | | | | | |
| C5 | Cell membrane hydrophobic | CCCC═CCC═CCC═CCC═CCCCCCC | 17 | 0.9 | 2.4 | 5 |
| | portion#3: Phosphatidylethanolamine | | | | | |
| | hydrophobic | | | | | |
| C6 | Cell membrane hydrophilic | COP(═O)(═O)OCC(OC(═O)C)COC═OC | 17 | 12 | 12 | 5 |
| | portion#1: Phosphatidylcholine | | | | | |
| | hydrophilic portion | | | | | |
| C7 | Cell membrane hydrophilic | C[N+](C)(C)CCOP([O—])(═O)OCC(OC(═O)C)COC(═O)C | 17 | 9.8 | 16 | 5 |
| | portion#2: Sphingomyelin | | | | | |
| | hydrophilic portion | | | | | |
| C8 | Cell membrane hydrophilic | C[N+](C)(C)CCOP([O—])(═O)OCC(NC(═O)C)C(O)C | 18 | 17 | 20 | 5 |
| | portion#3: Phosphatidylethanolamine | | | | | |
| | hydrophilic portion | | | | | |
| C9 | Cell membrane hydrophilic | O═C(O[C@@H][COP(O)(═O)OC[C@H](N)C(O)═O)COC(═O)CC)C [symbol] | 18 | 13 | 19 | 5 |
| | portion#4: Phosphatidylserine | | | | | |
| C10 | Water (1% soluble) | [H]O[H] | 15.1 | 17.1 | 16.9 | 18.1 |

[symbol]

indicates data missing or illegible when filed

The HSP is particularly preferably present outside of HSP spheres of one or more cell components of water, DNA and cholesterol, and more preferably present outside of HSP spheres of all of the cell components. The HSP is further preferably present outside of HSP spheres of one or more cell components, other than three components described above, selected from the group consisting of phosphatidycholine (hydrophobic moities 1, 2, hydrophilic moiety), phosphatidylethanolamine (hydrophobic moiety, hydrophilic moiety), sphingomyelin (hydrophilic moiety) and phosphatidylserine.

Examples of liquids having the compatible HSPs include the following liquids. Examples thereof also include mixed solvents of the following liquids that have HSPs outside of the HSP spheres.

TABLE 7

| No. | Solvent name | CAS No. | SMILES |
|---|---|---|---|
| 1 | Ethyl Butyl Ether | 626-81-8 | CCOCCCC |
| 2 | HCFC 225cb | 507-55-1 | ClC(F)(F)C(F)(F)C(F)([H])Cl |
| 3 | Perfluoro Dimethylcyclohexane | 374-77-8 | C1(C(C(C(C(C1(F)F)(F)F)(F)F)(C(F)(F)F)F)(F)F)(C(F)(F)F)F |
| 4 | Perfluoroheptane | 335-57-9 | FC(C(F)(F)C(F)(F)C(F)(F)F)(F)C(F)(F)C(F)(F)C(F)(F)F |
| 5 | Perfluoromethylcyclohexane | 355-02-2 | FC(C1(F)F)(C(F)(F)C(F)(F)C(F)(F)C1(F)F)C(F)(F)F |
| 6 | Tetraethylorthoalicate | 78-10-4 | CCO[Si](OCC)(OCC)OCC |
| 7 | HFC-43-10m [symbol] | 186496-42-8 | C(C(C(F)(F)F)F)(C(C(F)(F)F)(F)F)F |
| 8 | HFE 7000 | 375-03-1 | COC(F)(C(F)(C(F)(F)F)F)F |
| 9 | HFE 7500 | 297730-93-9 | CCOC(C(C(C(F)(F)F)(F)F)(F)F)(C(C(F)(F)F)(C(F)(F)F)F)F |
| 10 | HFE 8200 | NA | |
| 11 | OS-10 (Hexamethyldisiloxane) | 107-48-0 | C[Si](C)(C)O[Si](C)(C)C |
| 12 | OS-20 (Octamethyldisiloxane) | 107-51-7 | C[Si](C)(C)O[Si](C)(C)O[Si](C)(C)C |
| 13 | OS-30 (Decamethyltetrasiloxane) | 141-82-8 | C[Si](C)(C)O[Si](C)(C)O[Si](C)(C)O[Si](C)(C)C |
| 14 | Perfluorohexane (PFC 5060) | 355-42-0 | FC(F)(C(F)(C(F)(C(F)(C(F)(C(F)(F)F)F)F)F)F)F |
| 15 | Octamethylcyclotetrasiloxane | 556-87-2 | C[Si]1(C)O[Si](C)(C)O[Si](C)(C)O[Si](C)(C)O1 |
| 16 | Decamethylcyclopentasiloxane | 541-02-6 | [Si]1(C)(C)O[Si](C)(C)O[Si](C)(C)O[Si](C)(C)O[Si](O1)(C)C |
| 17 | 2,2,3,3,3-Pentafluoropropyl Methyl Ether | 378-18-5 | COCC(F)(F)C(F)(F)F |
| 18 | Methyl 1,1,2,2,3,3-Hexafluoropropyl Ether | 163620-20-2 | COC(F)(C(F)(C(F)F)F)F |
| 19 | 1,1,2,3,3,3-Hexafluoropropyl Methyl Ether | 382-34-3 | COC(F)(C(C(F)(F)F)F)F |
| 20 | Difluoromethyl 2,2,3,3-Tetrafluoropropyl Ether | 35042-99-0 | FC(C(COC(F)F)(F)F)F |
| 21 | Difluoromethyl 2,2,3,3,3-Pentafluoropropyl Ether | 58853-81-2 | FC(OCC(C(F)(F)F)(F)F)F |
| 22 | Trifluoromethyl 2,2,3,3-Tetrafluoropropyl Ether | 1883-81-4 | FC(OCC(F)(C(F)F)F)(F)F |
| 23 | 1-Trifluoromethyl-2,2,2-Trifluoroethyl Methyl Ether | 13171-18-1 | COC(C(F)(F)F)C(F)(F)F |
| 24 | 1,1,1,3,3,3-Hexafluoro-2-(Difluoromethoxy)Propane | 26103-08-2 | FC(F)(C(C(F)(F)F)OC(F)F)F |
| 25 | 1,1,2,2,2-Pentafluoroethyl Ethyl Ether | 22052-81-9 | CCOC(F)(C(F)(F)F)F |
| 26 | 2,2-Difluoroethyl 1,1,2,2-Tetrafluoroethyl Ether | 50807-77-7 | FC(COC(F)(C(F)F)F)F |
| 27 | 1,1,2-Trifluoroethyl 2,2,2-Trifluoroethyl Ether | 25449-81-0 | FC(OCC(F)(F)F)(CF)F |
| 28 | 1,1,2,2-Tetrafluoroethyl 2,2,2-Trifluoroethyl Ether | 408-78-0 | FC(OCC(F)(F)F)(C(F)F)F |
| | As-3000 | | |
| 29 | Pentafluoroethyl 2,2-Difluoroethyl Ether | 171182-95-8 | FC(OCC(F)F)(C(F)(F)F)F |
| 30 | Propane, 1,1,1,2,2-Pentafluoro-3-(1,1,2,2- | 50807-74-4 | FC(COC(F)(C(F)F)F)(C(F)(F)F)F |
| | Tetrafluoroethoxy)- | | |
| 31 | Propane, 2-(Difluoromethoxymethyl)-1,1,1,3,3,3- | 382-28-3 | COC(C(C(F)(F)F)C(F)(F)F)(F)F |
| | Hexafluoro- | | |
| 32 | 2,2,3,4,4,4-Hexafluorobutyl Difluoromethyl Ether | 69948-46-6 | FC(OCC(C(C(F)(F)F)F)(F)F)F |
| 33 | Butane, 1,1,1,2,3,3-Hexafluoro-4-(Trifluoromethoxy)- | 69948-43-2 | FC(COC(F)(F)F)(C(C(F)(F)F)F)F |
| 34 | Butane, 1-Ethoxy-1,1,2,2,3,3,4,4,4-Nonafluoro- | 163702-05-4 | CCOC(C(C(C(F)(F)F)(F)F)(F)F)(F)F |
| 35 | Propane, 1,1,1,3,3,3-Hexafluoro-2,2-Dimethoxy- | 754-50-7 | FC(C(OC)(OC)C(F)(F)F)(F)F |
| 36 | 2,2-Bis(Trifluoromethyl)-1,3-Dioxolane | 1765-25-0 | C1COC(O1)(C(F)(F)F)C(F)(F)F |
| 37 | Butanoic Acid, Heptafluoro-, Methyl Ester | 356-24-1 | FC(F)(F)C(F)(F)C(F)(F)C(═O)OC |
| 38 | Propanoic Acid, 3,3,3-Trifluoro-2-(Trifluoromethyl)-, | 360-54-3 | FC(F)(F)C(C(═O)OC)C(F)(F)F |
| | Methyl Ester | | |
| 39 | Propendic Acid, Pentafluoro-, Ethyl Ester | 426-85-3 | CCOC(C(C(F)(F)F)(F)F)═O |
| 40 | Butanoic Acid, Heptafluoro-, Ethyl Ester | 358-27-4 | FC(F)(F)C(F)(F)C(F)(F)C(═O)OCC |
| 41 | 2-Pentanone, 1,1,1,3,3,4,4,5,5-Nonafluoro- | 50838-70-5 | O═C(C(C(F)(C(F)F)F)(F)F)C(F)(F)F |
| 42 | 3-Pentanone, 1,1,1,2,2,5,5,5-Octafluoro- | 61837-82-1 | O═C(CC(F)(F)F)C(F)(C(F)(F)F)F |
| 43 | 2-Pentanone, 3,3,4,4,5,5,5-Heptafluoro- | 355-17-9 | CC(C(F)(C(F)(C(F)(F)F)F)F)═O |
| 44 | 2-Butanone, 3,4,4,4-Tetrafluoro-3-(Trifluoromethyl)- | 80658-01-1 | CC(C(C(F)(F)F)(C(F)(F)F)F)═O |
| 45 | 2-Pentanone, 3,3,4,5,5,5-Hexafluoro- | 80249-67-4 | CC(C(F)(C(C(F)(F)F)F)F)═O |
| 46 | 3-Pentanone, 1,1,1,2,2-Pentafluoro- | 378-72-3 | C(C)C(C(C(F)(F)F)(F)F)═O |
| 47 | 3-Pentanone, 1,1,1,2,2,4,5,5,5-Nonafluoro-4- | 756-13-8 | O═C(C(C(F)(F)F)(C(F)(F)F)F)C(F)(C(F)(F)F)F |
| | (Trifluoromethyl)- | | |
| 48 | 3-Pentanone, 1,1,1,2,2,5,5,5-Octafluoro-4- | 61637-91-0 | O═C(C(C(F)(F)F)C(F)(F)F)C(F)(C(F)(F)F)F |
| | (Trifluoromethyl)- | | |
| 49 | 2-Hexanone, 1,1,1,3,3,4,4,5,5,6,6-Undecafluoro- | 42287-76-2 | O═C(C(C(F)(C(C(F)F)(F)F)F)(F)F)C(F)(F)F |
| 50 | 2-Hexanone, 3,3,4,4,5,5,6,6,6-Nonafluoro- | 678-18-2 | FC(F)(F)C(F)(F)C(F)(F)C(F)(F)C(C)═O |
| 51 | 2-Hexanone, 3,3,4,4,5,5,6,6-Octafluoro- | 93449-49-1 | CC(C(C(F)(C(C(F)F)(F)F)F)(F)F)═O |
| 52 | 3-Hexanone, 4,4,5,5,6,6,6-Heptafluoro- | 358-23-0 | O═C(C(C(C(F)(F)F)(F)F)(F)F)CC |
| 53 | 2-Pentanone, 1,1,1,5,5,5-Hexafluoro-4-Methyl- | 372-24-7 | O═C(CC(C(F)(F)F)C)C(F)(F)F |
| 54 | 2,2,2-Trifluoroethyl Trifluoroacetate | 407-38-5 | O═C(C(F)(F)F)OCC(F)(F)F |
| 55 | Methyl Pentafluoropropionate | 378-75-6 | COC(C(C(F)(F)F)(F)F)═O |
| 56 | Isopropyl Trifluoroacetate | 400-38-4 | CC(OC(C(F)(F)F)═O)C |
| 57 | 1,1,1,3,3,3-Hexafluoro Isopropyl Acrylate | 2180-89-6 | FC(F)(F)C(OC(═O)C═C)C(F)(F)F |
| 58 | Ethyl Perfluorooctanoate | 3108-24-5 | FC(F)(F)C(F)(F)C(F)(F)C(F)(F)C(F)(F)C(F)(F)C(F)(F)C(═O)OCC |
| 59 | 1,1,1,5,5,6,6,6-Octafluoro-2,4-Hexanedione | 20825-07-4 | FC(F)(F)C(═O)CC(═O)C(F)(F)C(F)(F)F |
| 60 | Heptafluoropropyl-1,2,2,2-Tetrafluoroethyl Ether | 3330-15-2 | FC(OC(F)(F)C(F)(F)C(F)(F)F)C(F)(F)F |

[symbol]

indicates data missing or illegible when filed

TABLE 8

| No. | Solvent name | CAS No. | SMILES |
|---|---|---|---|
| 61 | Perfluorotripropylamine | 338-83-0 | FC(F)(F)C(F)(F)C(F)(F)N(C(F)(F)C(F)(F)C(F)(F)F)C(F)(F)C(F)(F)C(F)(F)F |
| 62 | 2-Pyran, 2,2,3,3,4,4,5,5,6,6-Decafluorotetrahydro- | 355-79-3 | FC(C(C(OC(C(F)1F)(F)F)(F)F)(F)F)1F |
| 63 | Furan, 2,2,3,3,4,4,5-Heptafluorotetrahydro-5- | 358-48-9 | FC(F)(OC1(F)C(F)(F)C(F)(F)F)C(F)(F)C1(F)F |
| 64 | Furan, 2,2,3,4,5,5-Hexafluorotetrahydro-3,4- | 68088-53-9 | FC1(C(F)(F)F)C(F)(C(F)(F)F)C(F)(F)OC(F)1F |
| 65 | Furan, 2,2,3,3,4,5,5-Heptafluorotetrahydro-4- | 61340-72-5 | FC1(F)C(F)(C(F)(C(F)(F)F)F)C(F)(F)OC(F)1F |
| 66 | Furan, 2,2,3,3,4,5-Hexafluorotetrahydro-4,5- | 61340-71-4 | FC1(F)C(F)(C(F)(F)F)C(F)(C(F)(F)F)OC(F)1F |
| 67 | Furan, 2,2,3,3,5,5-Hexafluorotetrahydro-4,4- | 61340-73-6 | FC1(F)C(C(F)(F)F)C(F)(F)F)C(F)(F)OC(F)1F |
| 68 | 2-Pyran, 2,2,3,3,4,4,5,6,6-Nonafluorotetrahydro- | 61340-74-7 | FC1(F)C(F)(F)C(F)(F)C(F)(C(F)(F)F)C(F)(F)O1 |
| | 5- | | |
| 69 | Furan, 2,2,3,4,4,5-Hexafluorotetrahydro-3,5- | 67408-02-4 | F[C@](O1)(C(F)(F)F)C([C@](F)(C(F)(F)F)C1(F)F)(F)F |
| | Bis(Trifluoromethyl)-, Trans- | | |
| 70 | 2-Pyran, 2,2,3,3,4,4,5,5,6-Nonafluorotetrahydro- | 358-47-8 | C1(C(C(OC(C1(F)F)(F)F)(C(F)(F)F)F)(F)F)(F)F |
| | 6- | | |
| 71 | Furan, 2,3,3,4,4,5-Hexafluorotetrahydro-2,5- | 59683-83-1 | FC1(C(F)(F)F)OC(F)(C(F)(F)F)C(F)(F)C(F)1F |
| 72 | Oxepane Dodecafluoro | 788-41-0 | FC1(C(C(F)(F)OC(F)(F)C(F)(F)C1(F)F)(F)F)F |
| 73 | Trimethyl(2-Heptafluoropropoxy-1,1,2- | f341 | C[Si](C)(C(F)(C(OC(F)(C(F)(C(F)(F)F)F)F)F)F)C |
| | Trifluoroethyl)Silane | | |
| 74 | Nonafluoro-Tert-Buthanol | 2378-02-1 | FC(F)(F)C(O)(C(F)(F)F)C(F)(F)F |
| 75 | Hexafluoro-Tert-Buthanol | 1515-14-8 | FC(F)(F)C(O)(C)C(F)(F)F |
| 76 | Nonafluoro-Cyclopentane | 378-65-8 | FC(C(F)(C(F)(C(F)1F)F)F)C1(F)F |
| 77 | 1,1,1,2,2,3,3-Heptafluoro-Pentane | 754-88-7 | CCC(C(C(F)(F)F)(F)F)(F)F |
| 78 | 2H-Heptafluoro-1,4-Dioxane | 34115-18-8 | FC(OC1F)(C(OC(F)1F)(F)(F)F |
| 79 | Difluoromethyl 1,1,2,3,3,3-Hexafluoropropyl | 58880-85-6 | FC(F)(C(C(OC(F)F)(F)F)F)F |
| | Ether | | |
| 80 | Fluoromethyl 1,1,2,3,3,3-Hexafluoropropyl | 60598-14-3 | FCOC(C(C(F)(F)F)F)(F)F |
| | Ether | | |
| 81 | 1-Difluoromethyl-1,2,2-Trifluoroethyl | 58899-47-9 | FC(C(F)F)(C(F)F)OC(F)F |
| | Difluoromethyl Ether | | |
| 82 | 1-Trifluoromethyl-2,2,2-Trifluoroethyl | 28523-88-8 | FCOC(C(F)(F)F)C(F)(F)F |
| | Fluoromethyl Ether | | |
| 83 | Bis(1,1,2-Trifluoroethyl)Ether | 51100-29-9 | FC(OC(F)(CF)F)(CF)F |
| 84 | 1,1,2-Trifluoro-2-Trifluoromethoxy-1- | 996-56-5 | COC(C(OC(F)(F)F)F)(F)F |
| | Methoxyethane | | |
| 85 | Perfluor-Tert-Buthylamine | 2808-82-8 | FC(F)(F)C(N)(C(F)(F)F)C(F)(F)F |
| 86 | Decafluoro-1-Trifluoromethyl-Piperidine | 359-71-7 | C1(C(C(N(C(C1(F)F)(F)F)C(F)(F)F)(F)F)(F)F)(F)F |
| 87 | Tris-Pentafluoroethyl-Amine | 359-70-8 | FC(F)(F)C(F)(F)N(C(F)(F)C(F)(F)F)C(F)(F)C(F)(F)F |
| 88 | 1,1,2,2,2-Pentafluoroethyl 1,1,2-Trifluoroethyl | f781 | FC(OC(F)(C(F)(F)F)F)(CF)F |
| | Ether | | |
| 89 | Ethyl 1,1,2,2,3,3,3-Heptafluoropropyl Ether | 22052-86-4 | CCOC(C(C(F)(F)F)(F)F)(F)F |
| 90 | Propane, 1-(2,2-Difluoroethoxy)-1,1,2,2,3,3,3- | 178310-28- | FC(F)(C(F)(C(OCC(F)F)(F)F)F)F |
| | Heptafluoro- | | |
| 91 | 2,2,2-Trifluoroethyl 1,1,2,2,3,3,3- | 142459-08- | FC(F)(C(F)(C(OCC(F)(F)F)(F)F)F)F |
| | Heptafluoropropyl Ether | | |
| 92 | 1,1,2,2,2-Pentafluoroethyl 2,2,3,3- | f786 | FC(OCC(F)(F)C(F)F)(C(F)(F)F)F |
| | Tetrafluoropropyl Ether | | |
| 93 | 1,1,2,2,2-Pentafluoroethyl 2,2,3,3,3- | 155853-44- | FC(C(COC(C(F)(F)F)(F)F)(F)(F)F)(F)F |
| | Pentafluoropropyl Ether | | |
| 94 | 2,2,3,3,4,4,4-Heptafluorobutyl Methyl Ether | 376-98-7 | COCC(F)(C(F)(C(F)(F)F)F)F |
| 95 | 1,1,2,2,3,3,3-Heptafluoropropyl 2,2,3,3- | f790 | FC(F)(C(F)F)COC(C(F)(C(F)(F)F)F)(F)F |
| | Tetrafluoropropyl Ether | | |
| 96 | 1,1,2,2,3,3,3-Heptafluoropropyl 2,2,3,3,3- | f791 | FC(OCC(F)(C(F)(F)F)F)(C(F)(C(F)(F)F)F)F |
| | Pentafluoropropyl Ether | | |
| 97 | 1-Hexanol, 3,3,4,4,5,5,6,6,6-Nonafluoro- | 2043-47-2 | FC(F)(F)C(F)(F)C(F)(F)C(F)(F)CCO |
| 98 | Propane, 1,1,1,3,3,3-Hexafluoro-2- | 142371-60-8 | FC(C(C(F)(F)F)(C(F)(F)F)SC(F)(F)F)(F)F |
| | (Trifluoromethyl)-2- | | |
| | [symbol] (Trifluoromethyl)Thiol- | | |
| 99 | 2-Pentane, 1,1,1,3,4,4,5,5,5-Nonafluoro- | 1584-03-6 | FC(F)(F)C(C(F)(F)F)═C(F)C(F)(F)C(F)(F)F |
| | 2-(Trifluoromethyl)- | | |
| 100 | 1-Hexane, 3,3,4,4,5,5,6,6,6-Nonafluoro- | 18430-93-4 | FC(F)(F)C(F)(F)C(F)(F)C(F)(F)C═C |
| 101 | 2H-Cyclopenta[B]Furan, | 72825-02-1 | FC1(F)C(F)(F)OC2(F)C(F)1C(F)(F)C(F)(F)C(F)2F |
| | Dodecafluorohexahydro- | | |
| 102 | 2H-Pyran, 2,2,3,3,4,4,5,6,6- | 67405-89-8 | FC1(F)OC(F)(F)C(F)(C(F)(C(F)(F)F)F)C(F)(F)C(F)1F |
| | Nonafluorotetrahydro-5-(Pentafluoroethyl)- | | |
| 103 | Furan, 2,2,3,3,4,5,5-Heptafluoro-4- | 67405-98-8 | FC1(C(F)(C(C(F)(F)F)(F)F)F)C(F)(F)C(F)(F)OC(F)1F |
| 104 | 2H-Pyran, 2,2,3,4,4,5,5,6- | 68083-03-6 | FC1(F)OC(F)(C(F)(F)F)C(F)(F)C(F)(F)C(C(F)(F)F)1F |
| | Octafluorotetrahydro-3,6- | | |
| | Bis(Trifluoromethyl)- | | |
| 105 | Furan, 2,2,3,4,4,5-Hexafluorotetrahydro- | 67405-84-1 | F[C@]1(C(F)(C(F)(F)F)F)C([C@@](F)(C(F)(F)F)OC(F)1F(F)F |
| | 3-(Pentafluoroethyl)-5- | | |
| | (Trifluoromethyl)-, Cis- | | |
| 106 | Furan, 2,2,3,4,4,5-Hexafluorotetrahydro- | 67422-82-8 | F[C@@]1(C(F)(C(F)(F)F)F)C([C@@](F)(C(F)(F)F)OC(F)1F(F)F |
| | 3-(Pentafluoroethyl)-5- | | |
| | (Trifluoromethyl)-, Trans- | | |
| 107 | Furan, 2,2,3,4,4,5-Hexafluorotetrahydro- | 68063-14-9 | F[C@@]1(C(F)(F)F)C([C@@](F)(C(F)(C(F)(F)F)F)OC(F)1F(F)F |
| | 5-(Pentafluoroethyl)-3- | | |
| | (Trifluoromethyl)-, Trans- | | |
| 108 | Furan, 2,2,3,4,4,5-Hexafluorotetrahydro- | 68083-02-5 | F[C@]1(C(F)(F)F)C([C@@](F)(C(F)(C(F)(F)F)F)OC(F)1F)(F)F |
| | 5-(Pentafluoroethyl)-3- | | |
| | (Trifluoromethyl)-, Cis- | | |
| 109 | Furan, 2,2,3,4,4,5-Hexafluorotetrahydro- | 74942-11-3 | FC1(F)C(F)(F)C(F)(C(F)(C(F)(F)F)F)OC(C(F)(F)F)1F |
| | 2-(Pentafluoroethyl)-5- | | |
| | (Trifluoromethyl)- | | |
| 110 | 2H-Pyran, 2,2,3,3,4,4,5,5,6- | 377-81-1 | FC1(F)C(F)(F)C(F)(F)C(F)(F)C(F)(C(F)(C(F)(F)F)F)O1 |
| | Nonafluorotetrahydro-6-(Pentafluoroethyl)- | | |
| 111 | Benzofuran, Tetradecafluorooctahydro- | 55751-38-5 | FC1(C(F)(F)C(F)(F)C(F)(F)C(F)2F)C2(F)OC(F)(F)C(F)1F |
| 112 | 2H-Cyclopenta[B]Furan, | 74403-40-0 | FC1(F)C(F)(C(F)(F)F)C(C(F)(F)C(F)(F)C(F)2F)(F)C2(F)O1 |
| | 2,2,3,3A,4,4,5,5,6,6,6A- | | |
| | Undecafluorohexahydro-3-(Trifluoromethyl)- | | |
| 113 | Furan, 2,2,3,3,4,4,5- | 335-38-4 | FC(F)(OC1(F)C(F)(F)C(F)(F)C(F)(F)C(F)(F)F)C(F)(F)C1(F)F |
| | Heptafluorotetrahydro-5-(Nonafluorobutyl)- | | |
| 114 | 2H-Pyran, 2,2,3,3,4,4,5,5,6-Nonafluoro-6- | 335-35-3 | FC(F)(OC(C1(F)F)(F)C(F)(F)C(F)(F)C(F)(F)F)C(F)(F)C1(F)F |
| | (Heptafluoropropyl)Tetrahydro- | | |
| 115 | Furan, 2,2,3,4,4,5-Hexafluorotetrahydro-3,5- | 68083-10-6 | FC1(C(F)(C(F)(F)F)F)C(F)(F)C(F)(C(F)(C(F)(F)F)F)C(F)(F)O1 |
| 116 | Furan, 2,2,3,3,4,5,5- | 646-65-5 | FC1(F)C(F)(F)C(F)(C(C(F)(C(C(F)(F)F)(F)F)F)(F)F)C(F)(F)O1 |
| | Heptafluorotetrahydro-4-(Nonafluorobutyl)- | | |
| 117 | 2H-Pyran, 2,2,3,3,4,4,5,6,6-Nonafluoro-5- | 801-28-3 | FC1(F)C(F)(F)C(F)(F)C(F)(C(F)(C(C(F)(F)F)(F)F)F)C(F)(F)O1 |
| | (Heptafluoropropyl)Tetrahydro- | | |
| 118 | Furan, 2,2,3,4,4,5-Hexafluoro-3- | 68083-08-1 | FC1(C(F)(F)F)C(F)(F)C(F)(C(F)(C(F)(C(F)(F)F)F)F)C(F)(F)O1 |
| | (Heptafluoropropyl)Tetrahydro-5- | | |
| | (Trifluoromethyl)- | | |
| 119 | Furan, 2,2,3,4,4,5-Hexafluoro-5- | 68083-15-0 | F[C@](OC([C@](F)1C(F)(F)F)(F)F)(C(F)(C(F)(C(F)(F)F)F)F)C1(F)F |
| | (Heptafluoropropyl)Tetrahydro-3- | | |
| | (Trifluoromethyl)-, Trans- | | |
| 120 | Furan, 2,2,3,4,4,5-Hexafluoro-5- | 68083-05-8 | F[C@](OC([C@@](F)1C(F)(F)F)(F)F)(C(F)(C(F)(C(F)(F)F)F)F)C1(F)F |
| | (Heptafluoropropyl)Tetrahydro-3- | | |
| | (Trifluoromethyl)-, Cis- | | |

[symbol]

indicates data missing or illegible when filed

TABLE 9

| No. | Solvent name | CAS No. | SMILES |
|---|---|---|---|
| 121 | Furan, 2,3,3,4,4,5-Hexafluoro-2- | 74842-12-4 | FC1(C(F)(C(F)(C(F)(F)F)F)F)C(F)(F)C(F)(F)C(F)(C(F)(F)F)O1 |
| | (Heptafluoropropyl)Tetrahydro-5- | | |
| | (Trifluoromethyl)- | | |
| 122 | Furan, Tetrahydro-2-[2,2,2-Trifluoro- | 73416-03-2 | FC(C(C(F)(F)F)(C(F)(F)F)C1CCCO1)(F)F |
| | 1,1-Bis(Trifluoromethyl)Ethyl]- | | |
| 123 | Butane, 1,1,1,2,2,3,3,4,4-Nonafluoro- | 559-29-5 | FC(OC(C(C(C(F)(F)F)(F)F)(F)F)(F)F)(F)F |
| | 4-(Trifluoromethoxy)- | | |
| 124 | Propane, 1,1,1,2,3,3-Hexafluoro-3- | 993-95-3 | FC(COC(C(C(F)(F)F)F)(F)F)(F)F |
| | (2,2,2-Trifluoroethoxy)- | | |
| 125 | Propane, 1,1,1,3,3,3-Hexafluoro-2- | 66670-22-2 | COC(C(F)(F)F)(C(F)(F)F)C(F)(F)F |
| | Methoxy-2-(Trifluoromethyl)- | | |
| 126 | Propane, 1,1,2,2-Tetrafluoro-3-(1,1,2,2- | 16627-68-2 | FC(COC(C(F)F)(F)F)(C(F)F)F |
| | Tetrafluoroethoxy)- | | |
| 127 | Butane, 1,1,1,2,3,4,4,4-Octafluoro-2- | 42551-02-0 | COC(C(C(F)(F)F)F)(C(F)(F)F)F |
| | Methoxy- | | |
| 128 | Propane, 1,1,1-Trifluoro-2-(1,1,2,2- | 50807-72-2 | [H]C(OC(C(F)F)(F)F)(C)C(F)(F)F |
| | Tetrafluoroethoxy)- | | |
| 129 | Propane, 1-Ethoxy-1,1,2,3,3,3-Hexafluoro- | 380-34-7 | CCOC(F)(C(C(F)(F)F)F)F |
| 130 | Propane, 1,1,2,3,3-Pentafluoro-1,3-Dimethoxy- | 758-62-3 | COC(F)(C(C(OC)(F)F)F)F |
| 131 | Propane, 3-Ethoxy-1,1,1-Trifluoro- | 406-96-4 | CCOCCC(F)(F)F |
| 132 | Butane, 1,1,1,2,2,3,3,4,4-Nonafluoro-4- | 71548-78-5 | FC(OC(C(F)(C(C(F)(F)F)(F)F)F)(F)F)(C(F)(F)F)F |
| | (Pentafluoroethoxy)- | | |
| 133 | Propane, 1,1′-Oxybis[1,1,2,2,3,3- | 356-82-7 | FC(F)(OC(F)(F)C(F)(F)C(F)(F)F)C(F)(F)C(F)(F)F |
| | Heptafluoro- | | |
| 134 | Ethane, 1,1,2,2-Tetrafluoro-1,2- | 356-70-7 | FC(OC(F)(C(OC(F)(C(F)(F)F)F)(F)F)F)(C(F)(F)F)F |
| | Bis(Pentafluoroethoxy)- | | |
| 135 | Propane, 1,1,1,2,3,3-Hexafluoro-3- | 1000-28-8 | FC(F)(C(C(OCC(F)(C(F)(F)F)F)(F)F)F)F |
| | (2,2,3,3,3-Pentafluoropropoxy)- | | |
| 136 | Propane, 1,1′-Oxybis[1,1,3,3,3- | 66711-94-2 | FC(OC(CC(F)(F)F)(F)F)(CC(F)(F)F)F |
| | Pentafluoro- | | |
| 137 | Propane, 1,1,1,2,3,3-Hexafluoro-3- | 65064-78-0 | FC(OCC(F)(C(F)F)F)(C(C(F)(F)F)F)F |
| | (2,2,3,3-Tetrafluoropropoxy)- | | |
| 138 | Propane, 2-(Ethoxydifluoromethyl)- | 380-30-9 | [H]C(C(OCC)(F)F)(C(F)(F)F)C(F)(F)F |
| | 1,1,1,3,3,3-Hexafluoro- | | |
| 139 | Butane, 2-Ethoxy-1,1,1,2,4,4,4-Heptafluoro- | 106893-05-4 | FC(OCC)(C(F)(F)F)CC(F)(F)F |
| 140 | Propane, 1,1,1,2,3,3-Hexafluoro-3- | 357-97-1 | CC(C)OC(F)(C(C(F)(F)F)F)F |
| | (1-Methylethoxy)- | | |
| 141 | Propane, 1,1′-Oxybis[3,3,3-Trifluoro- | 674-65-7 | FC(CCOCCC(F)(F)F)(F)F |
| 142 | Propane, 2-Ethoxy-1,1,1,2,3,3- | 682-30-6 | COC(OCC)(C(F)(F)F)C(F)(F)F |
| | Hexafluoro-2-Methoxy- | | |
| 143 | Butane, 1,1,1,3,3-Pentafluoro-4- | f1526 | COCC(C(C(F)(F)F)C(F)(F)F)(F)F |
| | Methoxy-2-(Trifluoromethyl)- | | |
| 144 | Octafluoro-n-Difluoro-n-Difluoromethyl- | 67212-89-9 | FC(OC1(F)F)(C(N(C(F)F)C(F)1F)(F)F)F |
| | Morpholine(S) | | |
| 145 | Octafluoro-4-Trifluoromethyl-Morpholine | 382-28-5 | FC(N(C(F)(F)F)C(F)(C(OC(F)1F)(F)F)F)1F |
| 146 | Hexafluoro-3-Pentafluoroethyl-Oxazolidine | 432-10-0 | FC(N(C(F)(C(F)(F)F)F)C1(F)F)(OC(F)1F)F |
| 147 | 2,2,3,3,4,4,4-Heptafluoro-Butylamine | 374-89-2 | NCC(F)(C(F)(C(F)(F)F)F)F |
| 148 | Methyl-(2,2,3,3,3-Pentafluoro-Propyl)- | 425-73-0 | CNCC(F)(F)C(F)(F)F |
| | Amine | | |
| 149 | Ethenamine, 2,2-Difluoro-N,N- | 176674-31-0 | FC(N(CC(F)F)C(F)(F)F)(F)F |
| | Bis(Trifluoromethyl)- | | |
| 150 | Fluoromethyl 1,1,2,2,3,3,3- | 184899-81-8 | FC(OCF)(C(F)(C(F)(F)F)F)F |
| | Heptafluoropropyl Ether | | |
| 151 | 1,1,1,2,2,3,3,4,4-Nonafluorohexane | 38436-17-8 | CCC(C(F)(C(F)(C(F)(F)F)F)F)(F)F |
| 152 | n-C4f9oc3h7 | 72372-80-6 | FC(OCCC)(C(C(F)(C(F)(F)F)F)(F)F)F |
| 153 | n-C5f11och3 | 181214-74-4 | COC(C(C(F)(C(C(F)(F)F)(F)F)F)(F)F)(F)F |
| 154 | n-C5f11oc2h5 | 181214-75-5 | CCOC(C(C(F)(C(C(F)(F)F)(F)F)F)(F)F)(F)F |
| 155 | Cf3cf(Ch2ch3)Ocf3 | f2329 | FC(F)(C(OC(F)(F)F)(CC)F)F |
| 156 | Cf3cf2ocf(Cf3)Cf2och3 | 202464-47-9 | COC(F)(C(OC(F)(C(F)(F)F)F)(C(F)(F)F)F)F |
| 157 | Cf3ch2cocf2cf3 | f2333 | O═C(C(F)(C(F)(F)F)F)OC(F)(F)F |
| 158 | (Cf3)2Chcocf2cf3 | f2334 | O═C(C(F)(C(F)(F)F)F)C(C(F)(F)F)C(F)(F)F |
| 159 | n-C3f7coch2ch3 | f2336 | CCC(C(F)(C(C(F)(F)F)(F)F)F)═O |
| 160 | (CF3)2CFCOCH3 | f2337 | O═C(C)C(C(F)(F)F)(C(F)(F)F)F |
| 161 | C5hf10no | f2339 | FC(N(C(F)1F)C(C(OC1(F)F)(F)F)(F)F)F |
| 162 | Methyl-<1,3,3,3-Tetrafluoro-2- | 350-53-2 | CO/C(F)═C(C(F)(F)F)/C(F)(F)F |
| | Trifluoromethyl-Propanyl>-Ether | | |
| 163 | Perfluoromethylcyclopentane | 1805-22-7 | C(F)(F)(F)C1(F)C(F)(F)C(F)(F)C(F)(F)C1(F)(F) |
| 164 | 1,2-Dichloro-1,1,2,3,3,3- | 681-97-2 | FC(F)(F)C(Cl)(F)C(F)(Cl)F |
| | Hexafluoropropane(F-2165a) | | |
| 165 | 1,2-Dichloro-1,2,3,3,3- | f2586 | FC(F)(F)C(C([H])(F)Cl)(F)Cl |
| | Pentafluoropropane(F-2258a) | | |
| 166 | Perfluoro-2-Methylpentane | 355-04-4 | FC(F)(F)C(F)(F)C(F)(F)C(C(F)(F)F)(F)C(F)(F)F |
| 167 | Perfluoro-3-Methylpentane | 865-71-4 | FC(C(F)(F)C(F)(F)F)(C(F)(F)C(F)(F)F)C(F)(F)F |
| 168 | Perfluoro-2,3-Dimethylbutane | 354-96-1 | FC(F)(F)C(F)(C(F)(F)F)C(F)(C(F)(F)F)C(F)(F)F |
| 169 | 1-Bromoperfluoroheptane | 375-88-2 | BrC(F)(F)C(F)(F)C(F)(F)C(F)(F)C(F)(F)C(F)(F)C(F)(F)F |
| 170 | 1-Bromoperfluorononane | 558-96-3 | FC(F)(F)C(F)(F)C(F)(F)C(F)(F)C(F)(F)C(F)(F)C(F)(F)C(F)(F)C(F)(Br)F |
| 171 | 1-Bromoperfluorooctane | 423-55-2 | FC(F)(F)C(F)(F)C(F)(F)C(F)(F)C(F)(F)C(F)(F)C(F)(F)C(F)(F)Br |
| 172 | 2-Chloro-1,1,2-Trifluoroethyl Ethyl Ether | 310-71-4 | CCOC(F)(F)C(F)Cl |
| 173 | 1,1,1,5,5,6,6,7,7,7-Decafluoro-2,4- | 20583-88-8 | FC(F)(C(CC(C(F)(C(F)(C(F)(F)F)F)F)═O)═O)F |
| | Heptanedione | | |
| 174 | 1,8-Dibromoperfluorooctane | 612-68-8 | BrC(F)(F)C(F)(F)C(F)(F)C(F)(F)C(F)(F)C(F)(F)C(F)(F)C(F)(F)Br |
| 175 | 1,3-Dichlorotetrafluoroacetone | 127-21-8 | O═C(C(F)(F)Cl)C(F)(F)Cl |
| 176 | Dodecafluorodimethylcyclobutane | 28577-00-1 | FC1(F)C(C(F)(F)F)(F)C(F)(F)C1(F)C(F)(F)F |
| 177 | 3,3,4,4,5,5,5-Heptafluoro-1-Pentane | 71164-40-4 | C═CC(F)C(F)(C(F)(F)F)F)F |
| 178 | Methyl Perfluorobutan-3-Oate | 20582-78-2 | O═C(OC)C(F)(F) C(F)═C(F) F |
| 179 | (Perfluorocyclohexyl)Methanol | 28788-68-3 | FC1(CO)C(F)(F)C(F)(F)C(F)(F)C(F)(F)C1(F)F |
| 180 | Perfluorod [symbol] | 306-94-5 | FC(C1(F)F)(C(C(F)(F)C(F)(F)C(F)(F)C2(F)F)(F)C2(F)C(F)(F)C1(F)F)F |

[symbol]

indicates data missing or illegible when filed

TABLE 10

| No. | Solvent name | CAS No. | SMILES |
|---|---|---|---|
| 181 | 1H,1H,2H-Perfluoro-1-Decane | 21652-58-4 | C═CC(F)(F)C(F)(F)C(F)(F)C(F)(F)C(F)(F)C(F)(F)C(F)(F)C(F)(F)F |
| 182 | Perfluoro-1,3-Dimethylcyclohexane | 336-27-3 | FC(F)(C(C(F)(C(F)(C1(F)F)C(F)(F)F)F)(C(F)(C1(F)F)F)F)F |
| 183 | 1H-Perfluoroheptane | 27213-61-2 | [H]C(F)(F)C(F)(F)C(F)(F)C(F)(F)C(F)(F)C(F)(F)C(F)(F)F |
| 184 | 1H,1H-Perfluoro-1-Heptanol | 375-82-6 | FC(F)(F)C(F)(F)C(F)(F)C(F)(F)C(F)(F)C(F)(F)CO |
| 185 | Perfluoroheptane-1 | 355-63-5 | FC(F)(F)C(F)(F)C(F)(F)C(F)(F)C(F)(F) C(F)═C(F) F |
| 186 | 8H-Perfluorohexane | 355-37-3 | FC(F)(F)C(F)(F)C(F)(F)C(F)(F)C(F)(F)C(F)(F)[H] |
| 187 | Perfluoro-1-Methyldecatin | 306-92-3 | FC12C(C(F)(F)C(F)(F)C(F)(F)C2(F)C(F)(F)F)(F)C(F)(F)C(F)(F)C(F)(F)C1(F)F |
| 188 | Perfluorooctane(S) | 307-34-6 | FC(F)(F)C(F)(F)C(F)(F)C(F)(F)C(F)(F)C(F)(F)C(F)(F)C(F)(F)F |
| 189 | 1H,1H-Perfluoro-1-Octanol | 307-30-2 | FC(F)(F)C(F)(F)C(F)(F)C(F)(F)C(F)(F)C(F)(F)C(F)(F)CO |
| 190 | 1H,1H,2H,2H-Perfluorooctanol | 647-42-7 | FC(F)(F)C(F)(F)C(F)(F)C(F)(F)C(F)(F)C(F)(F)CCO |
| 191 | Perfluorooctane-1 | f2810 | FC(F)═C(C(C(C(C(C(C(F)(F)F)(F)F)(F)F)(F)F)(F)F)(F)F)F |
| 192 | 1H,1H,2H-Perfluoro-1-Octane | 25291-17-2 | C═CC(F)(F)C(F)(F)C(F)(F)C(F)(F)C(F)(F)C(F)(F)F |
| 193 | Perfluoropiporidine | 535-77-1 | FC(C(F)(C(F)(C1(F)F)F)F)(C(F)(N1F)F)F |
| 194 | Perfluorooctyl Iodide | 507-63-1 | FC(C(F)(F)C(F)(F)C(F)(F)I)(F)C(F)(F)C(F)(F)C(F)(F)C(F)(F)F |
| 195 | 1,3-Dichlorohexafluoropropane (F-216) | 683-01-1 | FC(Cl)(F)C(F)(F)C(Cl)(F)F |
| 196 | Perfluorononane | 376-96-2 | FC(F)(C(F)(C(F)(C(F)(C(F)(C(F)(C(F)(C(F)(C(F)(F)F)F)F)F)F)F)F)F)F |
| 197 | C10f22 | 307-45-8 | FC(F)(F)C(F)(F)C(F)(F)C(F)(F)C(F)(F)C(F)(F)C(F)(F)C(F)(F)C(F)(F)C(F)(F)F |
| 198 | Cf3cf(Ocf3)Ch2chf2 | f2000 | FC(CC(F)F)(C(F)(F)F)OC(F)(F)F |
| 199 | Cf3cf(Ocf3)Ch2cf3 | f3001 | FC(OC(CC(F)(F)F)(C(F)(F)F)F)(F)F |
| 200 | 1,1-Bis-Difluoromethyl-1,2,2,2- | 267901-02-0 | FC(OC(OC(F)F)(C(F)(F)F)F)F |
| | Tetrafluoroethane | | |
| 201 | c-Cf2och(Cf3)Ocf2- | 269716-57-6 | FC(C(OC(F)1F)OC1(F)F)(F)F) |
| 202 | 2-Perfluoropropoxy-2,3,3,3- | 26537-88-2 | OCC(C(F)(F)F)(F)OC(F)(F)C(F)(F)C(F)(F)F |
| | Tetrafluoropropanol | | |
| 203 | 1,6-Divinyldodecafluorohexane | 1800-91-5 | FC(F)(C═C)C(F)(F)C(F)(F)C(F)(F)C(F)(F)C(F)(F)C═C |
| 204 | Perfluoro-5-Methylhexyl Iodide | 3456-8-6 | C(F)(F)C(F)(F)C(F)(F)C(F)(F)C(F)(C(F)(F)F)C(F)(F)F |
| 205 | Perfluoro-7-Methyloctyl Iodide | 865-77-0 | FC(F)(C(C(F)(F)F)(C(F)(C(F)(C(F)(C(F)(C(F)(C(F)(I)F)F)F)F)F)F)F)F |
| 206 | 1,1,2-Trifluoro-2-Chloroethyl 2,2,2- | 25384-98-1 | FC(COC(C(F)Cl)(F)F)(F)F |
| | Trifluoroethyl Ether | | |
| 207 | Perfluoro-2,7-Dimethyloctane | 3021-83-4 | FC(F)(F)C(C(F)(F)F)(F)C(F)(F)C(F)(F)C(F)(F)C(F)(F)C(C(F)(F)F)(F)C(F)(F)F |
| 208 | 1,1-Difluoroethyl-1,1,2,2-Tetrafluoroethyl | f4051 | CC(OC(F)(C(F)F)F)(F)F |
| | Ether | | |
| 209 | 1-(1,1-Difluoro-Ethoxy)-1,1,2,2,3,3,3- | f4055 | CC(OC(C(C(F)(F)F)(F)F)(F)F)(F)F |
| | Heptafluoropropane | | |
| 210 | Heptafuluoropropyl-1,2,2-Trifuluoroethyl Ether | f4056 | FC(C(OC(C(F)F)F)(F)F)(C(F)(F)F)F |
| 211 | 1,1,2,2,3,3,4,4-Octafluoro-5-Methoxypentane | f4057 | COCC(C(C(F)(C(F)F)F)(F)F)(F)F |
| 212 | Perfluoro(Isopentyl)Methylether | 203783-56-8 | COC(C(F)(C(C(F)(F)F)(C(F)(F)F)F)F)(F)F |
| 213 | Ethyl Tris(Trifluoromethyl)Methyl Ether | f4060 | CCOC(C(F)(F)F)(C(F)(F)F)C(F)(F)F |
| 214 | 2,2,4,4-Tetrafluoro-6,7-Bis(Trifluoromethyl)-3- | 180321-07-3 | FC(C1C(C(F)(F)F)C2C1C(OC(F)2F(F)F)(F)F |
| | Oxabicyclo[3.2.0]Heptane | | |
| 215 | 1,1,1,2,2-Pentafluoro-3-(2,2,3,3,3-Pentafluoro- | 1422-73-7 | FC(F)(C(COCOCC(F)(C(F)(F)F)F)(F)F)F |
| | Propoxymethoxy)-Propane | | |
| 216 | 1,1,1,3,3,3-Hexafluoro-2-(2,2,2-Trifluoro-1- | 184039-81-1 | FC(C(OCOC(C(F)(F)F)C(F)(F)F)C(F)(F)F)(F)F |
| | Trifluoromethylethoxymethoxy)Propane | | |
| 217 | 1-(2,2,2-Trifluoroethoxy)-2-Trifluoromethoxy- | f4075 | FC(OCC(F)(F)F)(C(OC(F)(F)F)F)F |
| | 1,1,2-Trifluoroethane | | |
| 218 | 1-(2,2,3,3,3-Pentafluoropropoxy)-2- | 226705-06-2 | FC(COC(C(OC(F)(F)F)F)(F)F)(C(F)(F)F)F |
| | Trifluoromethoxy-1,1,2-Trifluoroethane | | |
| 219 | 1,1,1,3,3,3-Hexafluoro-2-(1,1,2-Trifluoro-2- | 226705-05-1 | FC(OC(C(F)(F)F)C(F)(F)F)(C(OC(F)(F)F)F)F |
| | Trifluoromethoxyethoxy)Propane | | |
| 220 | Propane, 1,1,1,2,3,3-Hexafluoro-3-Methoxy-2- | 104159-55-9 | COC(C(OC(F)(F)F)(C(F)(F)F)F)(F)F |
| | (Trifluoromethoxy)- | | |
| 221 | 1-Ethoxy-1,1,2,3,3,3-Hexafluoro-2- | f4079 | CCOC(C(OC(F)(F)F)(C(F)(F)F)F)(F)F |
| | Trifluoromethoxypropane | | |
| 222 | 1-Ethoxy-1,1,2-Trifluoro-2- | 228705-03-9 | CCOC(C(OC(F)(F)F)F)(F)F |
| | Trifluoromethoxyethane | | |
| 223 | 1,1,2,2-Tetrafluoro-1,2-Bis(2,2,2- | f4081 | FC(OCC(F)(F)F)(C(OCC(F)(F)F)(F)F)F |
| | Trifluoroethoxy)Ethane | | |
| 224 | 5-Trifluoromethyl-3,3,4,4,5,6,6,6- | 244616-87-3 | O═C(C)C(C(C(C(F)(F)F)(C(F)(F)F)F)(F)F)(F)F |
| | Octafluorohexane-2-On | | |
| 225 | 4,4,5,5,6,6,7,7,7-Nonafluoro-3-Heptanone | 296280-00-7 | O═C(CC)C(C(C(C(F)(F)F)(F)F)(F)F)(F)F |
| 226 | 4,4,5,5,6,6,7,7,8,8,8-Undecafluoro-3-Octenone | f4088 | CCC(C(C(C(C(F)(C(F)(F)F)F)(F)F)(F)F)(F)F)═O |
| 227 | 3,3,4,4,5,5,6,6,7,7,7-Undecafluoro-2- | 2708-07-8 | O═C(C)C(C(F)(C(C(F)(C(F)(F)F)F)(F)F)F)(F)F |
| | Heptanone | | |
| 228 | Methyl Perfluoro (Pyrrolidinemethyl) Ketone | f4090 | O═C(C)C(N(C(F)1F)C(C(C(F)1F)(F)F)(F)F)(F)F |
| 229 | Methylfluoro(2-(N,N-Diamino)Ethyl)Ketone | f4093 | O═C(C)C(C(N(C(F)(F)F)C(F)(F)F)(F)F)(F)F |
| 230 | 1,1-Difluoro-1-(2,2,3,3,5,5,6,6- | f4094 | O═C(C)C(N(C(F)(C(OC(F)1F)(F)F)F)C1(F)F)(F)F |
| | Octafluoromorpholin-4-Yl)Acetone | | |
| 231 | 4-(Difluoromethyl)-2,6-Bis(Trifluoromethyl)- | 205876-74-0 | FC1(C(F)(F)F)OC(C(N(C(F)F)C(F)(F)(F)F)(C(F)(F)F)F |
| | 2,3,3,5,5,6-Hexafluoromorpholine | | |
| 232 | Ethyl 5H-Octafluoropentanoate | 2795-50-8 | O═C(OCC)C(F)(C(C(C(F)F)(F)F)(F)F)F |
| 233 | Ethyl 7H-Dodecafluoroheptanoate | 42287-85-4 | CCOC(C(F)(C(F)(C(F)(C(F)(C(F)(C(F)F)F)F)F)F)F)═O |
| 234 | Perfluorocyclohexane | 355-88-0 | FC(C(F)(C(F)(C1(F)F)F)F)(C(F)(C1(F)F)F)F |
| 235 | 1,5-Dichloro-1,1,3,3,5,5-Hexamethyltrisiloxane | 3582-71-6 | C[Si](C)(O[Si](C)(C)Cl)O[Si](C)(C)Cl |
| 236 | Dodecamethylcyclohexasiloxane | 540-97-6 | O1[Si](O[Si](O[Si](O[Si](O[Si](O[Si]1(C)C)(C)C) |
| | | | (C)C)(C)C)(C)C)(C) |
| 237 | Tetradecamethylhexasiloxane | 107-52-8 | C[Si](C)(C)O[Si](C)(C)O[Si](C)(C)O[Si](C)(C)O[Si](C) |
| | | | (C)O[Si](C)(C)C |
| 238 | Eicosamethylnonasiloxane | 2852-13-3 | C[Si](C)(C)O[Si](C)(C)O[Si](C)(C)O[Si](C)(C)O[Si](C) |
| | | | (C)O[Si](C)(C)O[Si](C)(C)O[Si](C)(C)O[Si](C)(C)C |
| 239 | Chlorotrimethylsilane | 75-77-4 | C[Si](C)(C)C |
| 240 | Hexamethylcyclotrisiloxane | 541-05-9 | C[Si]1(C[Si](C[Si](O1)(C)C)(C)C)C |
| 241 | Hexamethyldisilazene | 929-97-3 | [Si](C)(C)(C)N[Si](C)(C)C |

As described above, the biocompatible liquid according to the present disclosure may be defined to have a HSP compatible to a cell to which the liquid is applied. The HSP of the liquid may be identified on the basis of HSP threshold information and further of molar volume threshold information.

(Method for Determining HSP Threshold Information in Order to Identify Biocompatible Liquid)

The present disclosure may provide a method for determining an index for identifying a biocompatible liquid. The method may include obtaining biocompatibility and a HSP of one or more liquids to a veil to which the liquids are applied; and

defining, on the basis of the biocompatibility and the HSP, a HSP sphere serving as the HSP threshold information by a core (δD, δP, δH) in a HSP space associated with predetermined biocompatibility and an interaction radius R.

According to the present method, a HSP sphere serving as a biocompatible HSP space may be defined by biocompatibility and a HSP of a liquid and may be used as HSP threshold information. By determining threshold information as described above and obtaining a HSP for a liquid, biocompatibility of the liquid may be easily identified.

According to the present method, HSP threshold information may be determined and utilized in order to identify biocompatible liquid to various cell systems or to a specific cell.

The present method may further include a step of further obtaining a molar volume of one or more liquids, thereby allowing defining the HSP sphere on the basis of the molar volume. As described above, biocompatibility varies in a range of the molar volume of a liquid. Namely, in a certain range of the molar volume, biocompatibility of a liquid may in some cases vary significantly depending on HSP. Thus by defining a HSP sphere from the relationship between biocompatibility and HSP of one or more liquids in the certain range of the molar volume, highly accurate threshold information may be obtained.

For HSP threshold information and molar volume threshold information, embodiments described hereinabove may be appropriately applied.

The present method may include a step of obtaining, for one or more liquids, a HSP of a cell component of a cell to which the liquids are applied and a step of defining a HSP sphere serving as HSP threshold information by a core (δD, δP, δH) of the cell component in a HSP space and an interaction radius R one the basis of the HSP.

According to the present method, a HSP sphere of a cell component may be defined as a non-biocompatible HSP space and further utilized as threshold information for other HSPs. By determining threshold information as described above and obtaining a HSP for a liquid, biocompatibility of the liquid may be easily identified. According to the present method, HSP threshold information may be determined and utilized in order to identify biocompatible liquid to various cell systems or a specific cell.

Further, by combining with a HSP sphere serving as a biocompatible HSP space, biocompatibility may be identified with high accuracy.

The interaction radius of a cell component may be empirically determined or defined as preferably about 2 [J/cm³]^{1/2} or more and 10 [J/cm³]^{1/2} or less, more preferably 4 [J/cm³]^{1/2} or more and 6 [J/cm³]^{1/2} or less, still more preferably about 5 [J/cm³]^{1/2}.

For the cell component and HSP threshold information thereof, embodiments described hereinabove may be appropriately applied, respectively.

(Method for Screening Biocompatible Liquid)

According to the present disclosure, a method for screening a biocompatible liquid is provided. The screening method may include a step of identifying whether or not a test liquid has a HSP compatible to a cell to which the liquid is applied and/or a step of identifying whether or not the liquid has a molar volume compatible to the cell to which the liquid is applied. Whether or not the liquid has the compatible HSP in this context may be identified by applying the embodiments described hereinabove.

According to the present screening method, biocompatibility may be easily identified and a biocompatible liquid may be easily screened based on a HSP and/or molar volume of the test liquid substantially without experimentations. According to the present screening method, a liquid that is biocompatible in terms of essential significance to a cell may be screened.

According to the present screening method, molar volume threshold information, in addition to HSP threshold information obtained by directly bringing a test liquid into contact with a naked cell, may be used in order to identify whether or not a HSP of the test liquid is compatible. Thereby, a biocompatible liquid may be screened easily, substantially and with high accuracy.

(Method for Evaluating an Action of a Non-Hydrophilic Substance on Cells)

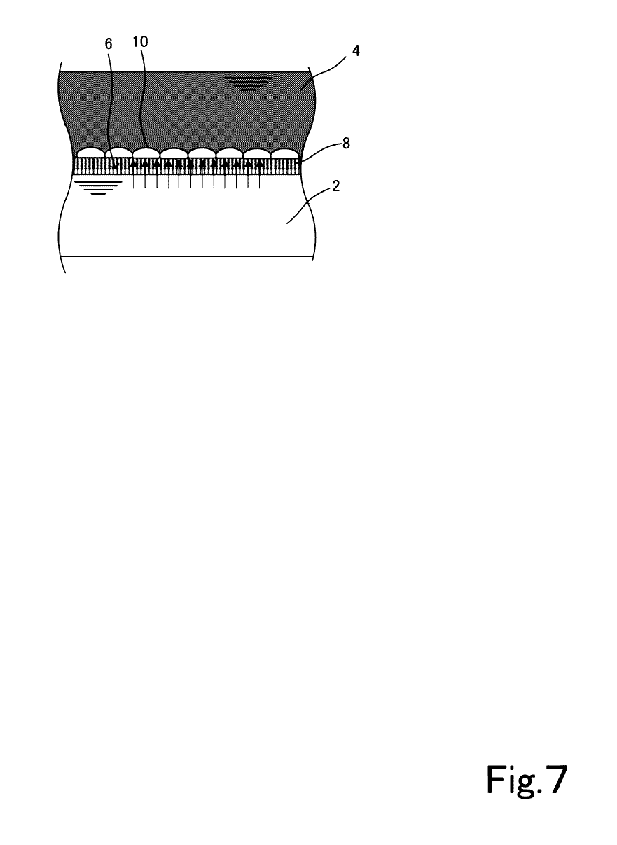

The evaluation method of the present disclosure may include, as shown in FIG. 7, a cultivation step of culturing a cell 10 while in contact with a first liquid 2, which is a hydrophilic liquid, and a second liquid 4. The present evaluation method may also include a step of evaluating the action of the non-hydrophilic substance on the cell 10 cultured as described above. In FIG. 7, the first liquid 2 is present at a lower layer and the second liquid 4 is present at an upper layer, and a support 8 is provided in the vicinity of the liquid-liquid interface 6.

The cell 10 is used for the present evaluation method. The cell 10 is selected according to the purpose of the evaluation method and the intended application.

(First Liquid)

The first liquid 2 is the hydrophilic liquid already described hereinabove. The first liquid 2 may be, but is not particularly limited to, for example, a liquid containing a nutritional component suitable for cultivation of the cell 10 to be used.

(Second Liquid)

The second liquid 4 is a non-hydrophilic liquid containing a non-hydrophilic substance. The present evaluation method is to evaluate an action of the non-hydrophilic substance on a cell by utilizing the non-hydrophilic liquid. One embodiment of the second liquid for evaluating the non-hydrophilic substance is the one in which the second liquid 4 is a non-hydrophilic liquid containing one or more non-hydrophilic substances as dispersed materials or solutes that are solid at the time of carrying out the present evaluation method. According to the embodiment, the second liquid 4 is a solution or dispersion (suspension or the like) of the non-hydrophilic substance in the non-hydrophilic liquid. As the non-hydrophilic substance is dissolved or uniformly dispersed in the non-hydrophilic liquid, it is possible to evaluate the action with high accuracy.

The non-hydrophilic liquid used for the embodiment is preferably a non-hydrophilic liquid having biocompatibility or low toxicity towards the cell used. Accordingly, the action of the non-hydrophilic substance may be more efficiently evaluated. Examples of the non-hydropilic liquid that may be mentioned by the inventors of the present invention include, in addition to the non-hydrophilic liquids already described in Tables 2 to 4 and 6 to 9, 1-ethoxy-1,1,2,2,3,3,4,4,4-nonafluorobutane, 1-ethoxy-1,1,2,3,3,3-hexafluoro-2-(trifluoromethyl)propane, 1,1,1,2,2,3,4,5,5,5-decafluoro-3-methoxy-4-(trifluoromethyl)pentane, 2-(difluoromethoxymethyl)-1,1,1,2,3,3,3-heptafluorobutane, 1,1,1,2,2,3,3,4,4-nonafluoro-4-methoxybutane and the like.

Another embodiment of the second liquid 4 is the one in which the second liquid 4 is a non-hydrophilic liquid obtained by mixing one or more non-hydrophilic substances that are liquids at the time of carrying out the present evaluation method. Namely, the second liquid 4 is a mixed liquid of a non-hydrophilic substance in the form of liquid and a non-hydrophilic liquid. As the non-hydrophilic substance in the form of liquid is miscible with the non-hydrophilic liquid, it is possible to evaluate the action with high accuracy. Also in the embodiment, the non-hydrophilic liquid, that serves as a medium, is preferably the one having more excellent biocompatibility or lower toxicity than the non-hydrophilic substance, as described above.

Another embodiment of the second liquid 4 is the one in which the second liquid 4 consists of one or more non-hydrophilic substances that are liquids at the time of carrying out the present evaluation method. Namely, the second liquid 4 is a liquid consisting of single non-hydrophilic substance or a mixed liquid consisting of two or more non-hydrophilic substances. As the cells 10 are exposed to a non-hydrophilic substance per se which is in the form of liquid, it is possible to evaluate the action with high accuracy.

(Cultivation Step)

The cultivation step in the present method is a step of culturing a cell 10 while in contact with the first liquid 2 and the second liquid 4. In the cultivation step, the cells may be cultured in any manner as far as the cells 10 are cultured while in contact as described above. For example, as shown in FIG. 7, the cells 10 may be cultured at the interface 6 between the first liquid 2 and the second liquid 4, by using a support 8 through which either or both of the first liquid 2 and the second liquid 4 can move as a scaffold, while in contact with the first liquid 2 and the second liquid 4.

(First Liquid Carrier and Second Liquid Carrier)

In the cultivation step, the first liquid 2 may be supplied by means of a first liquid carrier, which is not shown, and is configured so as to retain or allow flow of the first liquid 2. The second liquid 4 may be supplied by means of a second liquid carrier, which is not shown, and is configured so as to retain or allow flow of the second liquid.

The first liquid carrier may be a structure having a reservoir for reserving the first liquid 2 and a cavity such as a flow path through which the first liquid 2 can flow. The embodiment of the first liquid carrier has, but is not particularly limited to, a cavity having various forms including a wall material for shielding the first liquid 2. For example structures having the shape of containers, structures having the shape of tubes, structures having the shape of spheres, structures having any three-dimensional shapes may be used.

The first liquid carrier may be a gel structure that retains the first liquid 2. The gel structure retaining the first liquid 2 may be said to retain in itself the first liquid 2, and thus the first liquid carrier may have any arbitrary shape. Thus, the first liquid carrier may have the shape with the cavity as described above, or may be a solid structure such as the one with a sheet shape, with a columnar shape, with a spherical shape or an arbitrary three-dimensional shape. The gel structure may be the one generally called hydrogel Examples of the hydrogel include agar, agarose gel, collagen gel, alginate gel, 2-methacryloyloxyethyl phosphorylcholine (MPC) polymer and the like.

When the first liquid carrier is a gel structure, cells may be retained, for example, on the surface of the first liquid carrier. Namely, the first carrier in the form of gel may serve as a support 8 at the surface of the of the gel structure.

The first liquid carrier may be configured so as to be able to retain to retain the first liquid 2 or allow flow of the first liquid 2 and so that the first liquid 2 is supplied from the outside. Namely, the first liquid carrier may be configured so that the first liquid 2 is appropriately supplied via a first liquid supplying device provided outside of the first liquid carrier and a flow path system attached thereto. The first liquid carrier may include a discharge system of the first liquid 2.

The second liquid carrier may also have similar embodiments as the first liquid carrier. When the first liquid carrier is a structure having the shape of a container that has a cavity capable of reserving the first liquid 2 as well as the second liquid 4, the first liquid carrier also serves as the second liquid carrier. The second liquid carrier in the form of gel may be the one generally called organogel. The second liquid carrier preferably has a cavity for retaining a liquid so that the second liquid 4 can be directly brought into contact with cells 10.

(Support)

As shown in FIG. 7, the support 8 may include a structure that can retain a cell used for the present method. For example, the support 8 may be a porous structure. A porous structure has high surface area and can densely retain cells. Examples of the porous structure include but are not particularly limited to, porous glass or ceramics, porous plastics, porous polytetrafluoroethylene as well as laminates, interlaced structures, knitted structures and woven structures made up with fibers.

The support 8 which is a porous structure preferably has, as shown in FIG. 7, the porosity that allows formation of a sheet of cells on the surface of the support 8. When cells form a sheet on the support 8, the cells 10 may be cultured while the cells 10 per se prevent mutual transfer of the first liquid 2 and the second liquid 4 and form and maintain the interface 6 between the liquids. The prevention is based on the physical coverage and functional tight junctions of cells 10. The prevention of mutual transfer of the liquids does not necessarily mean complete prevention of mutual transfer of the liquids and may be the inhibition of mutual transfer of the liquids so as to make the present evaluation method useful.

The prevention of mutual transfer of the liquids by means of cells 10 per se is preferable because the interface 6 can be formed with the cells 10 regardless of specific gravities of the first liquid 2 and the second liquid 4, making the evaluation flexible. It is also preferable because in the following evaluation step, the activity of the cells and the action of the non-hydrophilic substance on the cells can be easily evaluated by detecting reduction in the extent of prevention. In view of this, the prevention of mutual transfer of the liquids by means of cells 10 may be evaluated or confirmed prior to the cultivation step.

The prevention of mutual transfer of liquids by means of cells may be evaluated with various methods such as measurement of the transepithelial electric resistance (TER).

In view of preparation of a cell sheet on the surface of a support 8, the support 8 which is a porous structure preferably has an average pore diameter of 10 μm or less. When the average pore diameter is higher than 10 μm, cells enter the pores, making it difficult to form a cell sheet on the surface of the support. The average pore diameter is preferably 5 μm or less and more preferably 3 μm or less. The average pore diameter may be measured with well-known methods. Typically, the average pore diameter may be calculated from measured values of pore diameters determined by acquiring more than one image of regions having a certain area by an electron microscope (TEM or SENM) and measuring pore diameters in the regions.

The support 8 may be a gel structure. A gel structure may generally retain cells on the surface thereof or inside thereof. The gel structure may be hydro gel retaining water or organogel retaining an organic solvent.

The support 8 may allow movement of either or both of the first liquid 2 and the second liquid 4. The support 8 allowing movement of a liquid encompasses both the movement of a liquid in the support 8 and the movement of a liquid from the outside of the support 8 through the support 8 to the outside of the support 8. Because the support 8 allows movement of a liquid, cells retained in the support 8 can be kept in contact with both of the first liquid 2 and the second liquid 4.

The support 8 preferably allows movement of the first liquid 2. Accordingly, regardless of the region of cells 10 retained in the support 8, a nutritional component may be supplied to the cells 10 via the first liquid 2.

The support 8 preferably allows movement of the second liquid 4. Accordingly, even when cells 10 are retained and cultured on the side of the first liquid 2 in the support 8, a nutritional component may be supplied via the support 8.

The support 8 preferably allows movement of the first liquid 2. The support 8 preferably allows movement of the first liquid 2 and movement of the second liquid 4. Accordingly, the support 8 may have appropriate porosity and surface properties. Further, it is also preferable that the support 8 allows movement of the first liquid 2 but does not allow movement of the second liquid 4. Accordingly, the interface 6 may be easily maintained. The support 8 that allows any of various movements may be obtained by a person skilled in the art using well-known materials.

A material for the support 8 may be appropriately selected from well-known hydrophilic and/or hydrophobic materials by taking for example, for example, cell retaining properties and movement of liquids through the support into consideration. For example, when cells are adherent cells, a well-known substance to which the cells adhere or a material containing or coated with the substance may be used. Examples of such an adherent and biological substance include, without particular limitation, collagen, fibronectin, vitronectin, laminin, nidogen fibrinogen, elastin, proteoglycan and the like. Examples also include well-known glass materials and plastic materials of which adherence to cells have been confirmed.

The support 8 may be fixed to the interface 6 or float on the interface 6 as far as the support 8 is present at the interface 6 between the first liquid 2 and the second liquid 4. The interface 6 between the first liquid 2 and the second liquid 4 is a concept encompassing the interface 6 and the vicinity of the interface 6.

The shape of the support 8 is not particularly limited and may be an arbitrary three-dimensional shape that is desired to confer to cells 10 that are subjected to evaluation. The shape may be, for example, a sheet, a tube, a column, a sphere (solid and hollow) and the like. The embodiment is not particularly limited as far as the support 8 is retained at the interface 6. The support 8 may have a shape according to the three-dimensional shape of cells 10 to be evaluated. A person skilled in the art can prepare various embodiments of the support according to the three-dimensional shape of the support 8, the shapes of the first liquid carrier 12 and the second liquid carrier 14 described hereinbelow, and the region where the support is formed with respect to the liquid carriers 12 and 14.

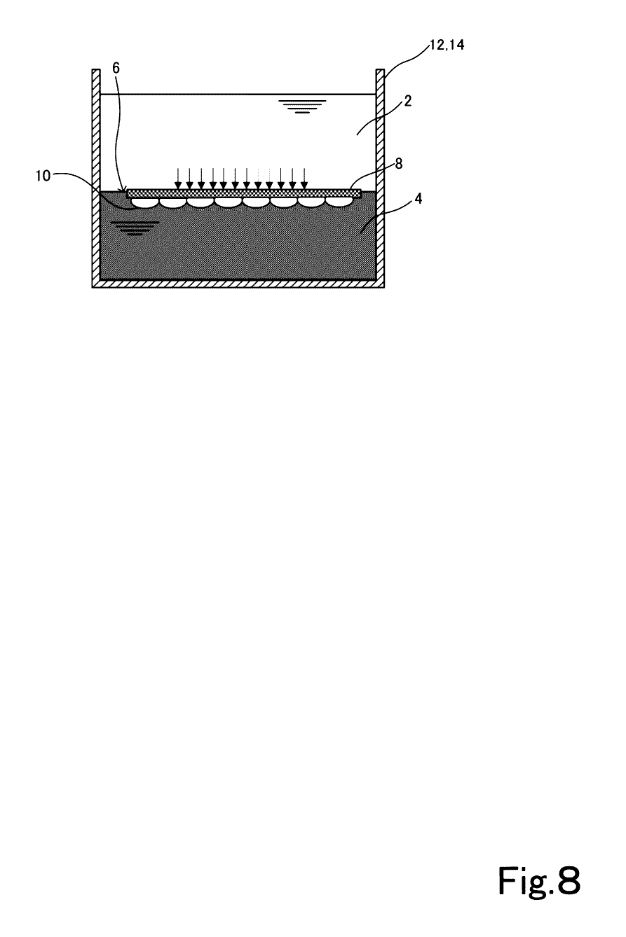

For example, without limitation, the cultivation step may be carried out for adherent cells in embodiments illustrated in FIG. 8 to FIG. 15. In the flowing embodiments, descriptions are made with the first liquid 2 that is a liquid medium mainly containing water and the second liquid 4 that is a solvent immiscible with the first liquid 2 and has a fluorocarbon structure or the like with a specific gravity higher than the first liquid 2.

### First Embodiment

As shown in FIG. 8, in the present embodiment, cells 10 retained on the lower surface of the support 8 are provided in the vicinity of the interface 6 of two phases, i.e. a lower layer of a second liquid 4 having a higher specific gravity and an upper layer of a first liquid 2 having a lower specific gravity. In the embodiment, the first liquid 2 and the second liquid 4 are collectively retained in or flow through structures having the shape of a container, namely, a first liquid carrier 12 and a second liquid carrier 14.

As shown in FIG. 8, in the present embodiment, the support floats in the vicinity of the vicinity of the interface 6. The support 8 allows movement of the first liquid 2, and allows cultivation of cells 10 while retaining the cells on the lower surface of the support 8, i.e. on the side of the second liquid 4. The cells 10 are exposed to the second liquid 4. Thereby it is possible to effectively evaluate the action of a non-hydrophilic substance in the second liquid 4 on the cells 10 and a sheet-shaped cell structure. Oxygen and the like are appropriately supplied to the cells 10. For example, nutritional components, oxygen and the like may be supplied from the first liquid 2. Oxygen may also be supplied via the second liquid 4.

When the support 8 allows movement of the second liquid 4, cells 10 may be retained and cultured on the side of the first liquid 2 on the support 8 because the second liquid 4 can reach the cells 10. When the support 8 allows movement of the first liquid 2 and the second liquid 4, cells 10 may be retained and cultured in the support 8.

### Second Embodiment

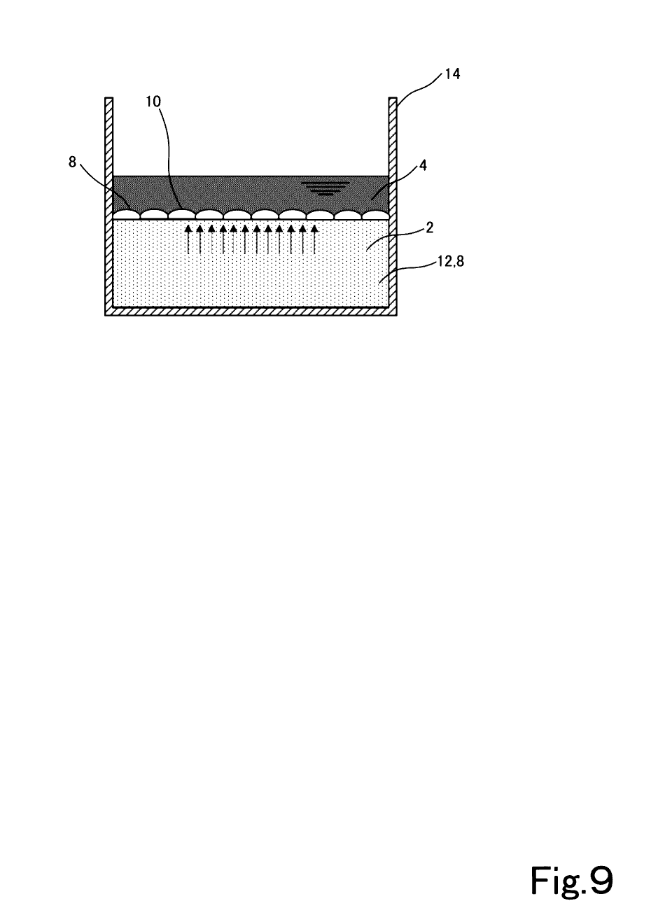

As town in FIG. 9, in the present embodiment, the first liquid carrier 12 for retaining the first liquid 2 is gel obtained by gelling the first liquid 2 with a gelling agent. The gel is porous and allows movement of the first liquid 2. Thus the first liquid carrier 12 also serves as a support 8 allowing movement of the first liquid 2.

The second liquid 4 is retained in a structure having the shape of a container, the second liquid carrier 14, and provided on the gel of the first liquid 2. Cells 10 are retained on the surface of the gel serving as the support 8 at the interface 6 between the gel of the first liquid 2 and the second liquid 4.

In the embodiment, cells 10 may be retained and cultured on the gel that serves as the support 8 and the first liquid carrier 12. The first liquid 2 is retained in the gel serving as the first liquid carrier and also can move in the gel Thereby the cells 10 are in contact with the first liquid 2, allowing supply of nutritional components to the cells. The cells 10 are exposed to the second liquid 4. Thereby it is possible to effectively evaluate the action of a non-hydrophilic substance in the second liquid 4 on the cells 10 and a sheet-shaped cell structure.

In the present embodiment, it is preferable that the first liquid carrier 12 does not allow movement of the second liquid or movement of the second liquid is prevented due to intercellular junctions of the cells 10. In the present embodiment, the first liquid 2 is in the form of gel, and thus the second liquid 4 having a higher specific gravity than the first liquid 2 may be provided in an upper layer.

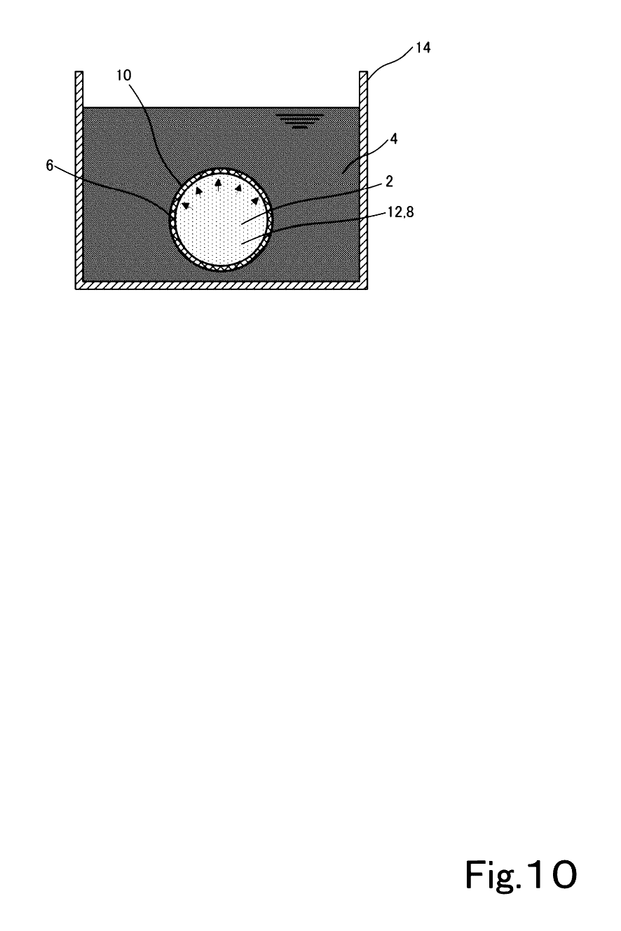

A modification of the second embodiment is shown in FIG. 10. As shown in FIG. 10, in the modification, a first liquid carrier 12 used is a gel structure having a desired three-dimensional shape. The first liquid carrier 12 has an outer surface that serves as a support 8 to retain cells. The first liquid carrier 12 is placed in a second liquid 4 retained in, for example, a second liquid carrier 14 in order to expose cells 10 to the second liquid 4.

According to the modification, it is possible to effectively evaluate the action of a non-hydrophilic substance in the second liquid 4 on the cells 10 and a cell structure having an outer shell with a desired three-dimensional shape. In the modification, the first liquid carrier 12 may be a three-dimensional cell structure which is constituted with a certain scaffold material and can accommodate cells not only on the surface thereof but also inside thereof.

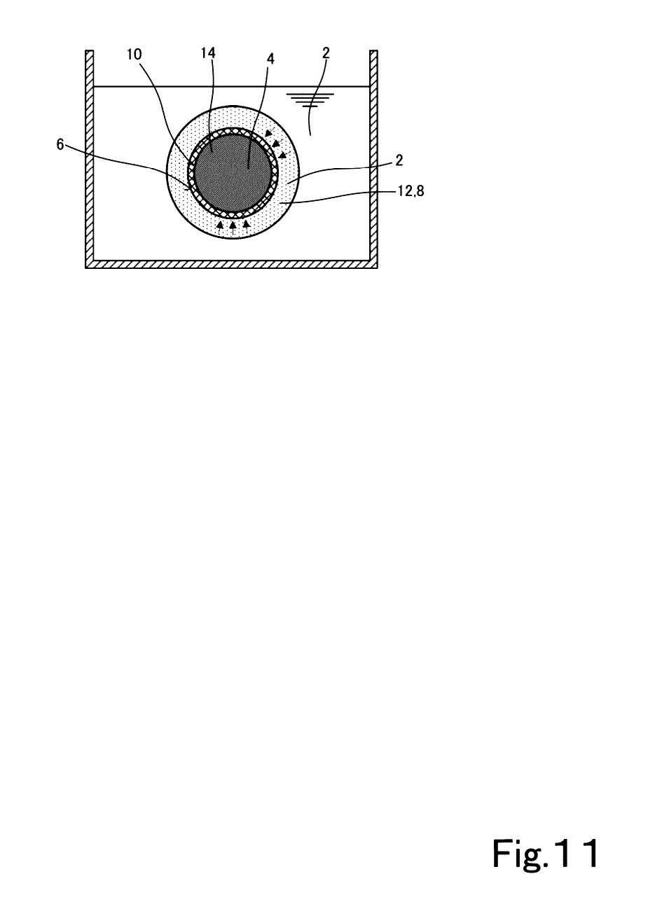

Another modification of the second embodiment is farther shown in FIG. 11. As shown in FIG. 11, in the modification, a first liquid carrier 12 used is a gel structure having a desired three-dimensional shape provided with a cavity of a second liquid carrier 14 that can retain a second liquid 4 or through which the second liquid 4 can flow. In the first liquid carrier 12, the inner surface of the cavity serves as the support 8 to retain cells. By retaining the second liquid 4 in the cavity of the first liquid carrier 12, cells 10 are exposed to the second liquid 4.

According to the modification, it is possible to effectively evaluate the action of a non-hydrophilic substance in the second liquid 4 on the cells 10 and a cell structure having a desired three-dimensional shape corresponding to the shape of the inner surface. In the modification, the first liquid carrier 12 may be a three-dimensional cell structure which is constituted with a certain scaffold material and can accommodate cells not only on the surface thereof but also inside thereof.

### Third Embodiment

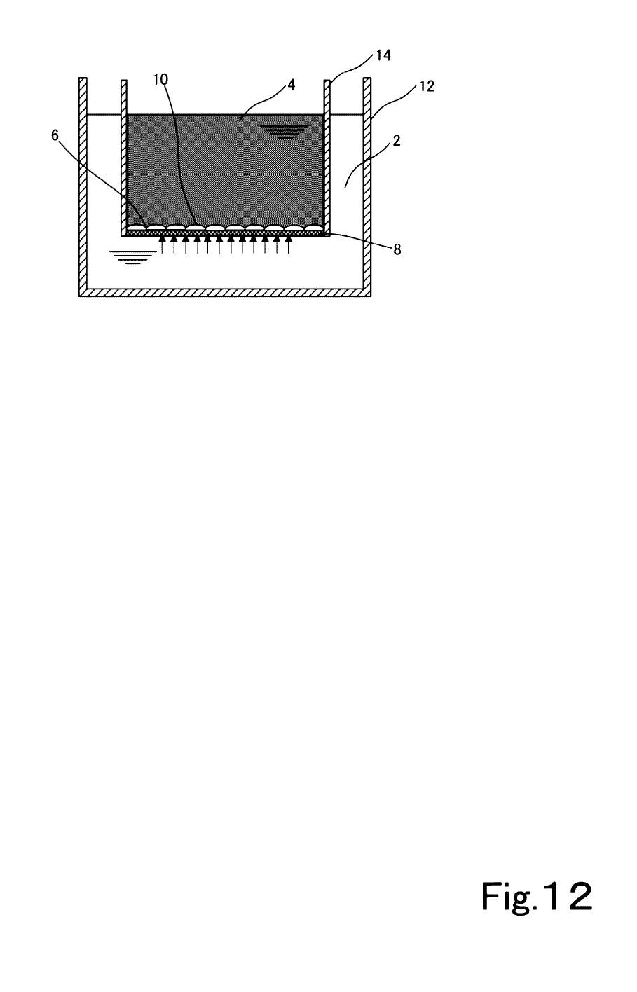

As shown in FIG. 12, in the present embodiment, the first liquid carrier 12 used is a structure having the shape of a container which can retain a first liquid 2 or through which the first liquid 2 can flow. The second liquid carrier 14 used is a structure having the shape of a container which can retain a second liquid 4 of through which the second liquid 4 can flow. The second liquid carrier 14 includes a bottom that serves as a support 8 allowing movement of the first liquid 2 and the second liquid 4. Cells 10 are retained on the surface of the support 8. The second liquid carrier 14 may be accommodated in a cavity of the first liquid carrier 12.

As shown in FIG. 12, the second liquid carrier 14 in which cells are retained on the support 8 is placed in the first liquid carrier 12 retaining the first liquid 2, and the second liquid is retained in the second liquid carrier 14. Thereby the support 8 may be provided at the interface 6 between the first liquid 2 and the second liquid 4 and cells 10 may be provided on the support 8.

When cells 10 are cultured in the embodiment, nutritional components are supplied to cells 10 while cells 10 are in contact with the first liquid 2 through the support 8. The cells 10 are also exposed to the second liquid 4. Thereby it is possible to effectively evaluate the action of a non-hydrophilic substance in the second liquid 4 on the cells 10 and a sheet-shaped cell structure.

In the present embodiment, the second liquid 4 having a higher specific gravity is in an upper layer, and thus it is preferable that the support 8 does not allow movement of the second liquid 4 or cells on the support 8 are configured to fill the support 8 in order to prevent downward movement of the second liquid 4.

### Fourth Embodiment

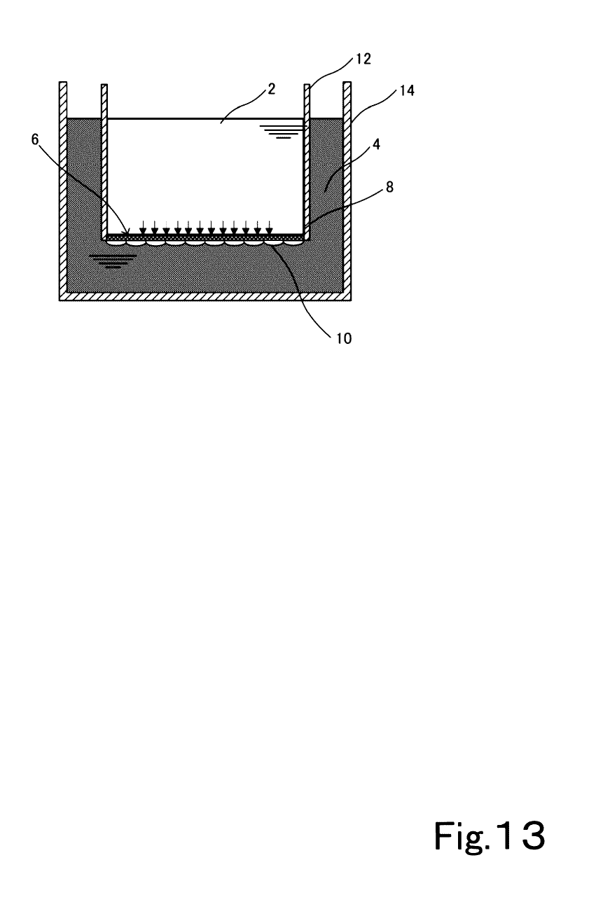

As shown in FIG. 13, in the present embodiment, a first liquid 2 and a second liquid 4 are interchanged from those in the third embodiment illustrated in FIG. 12. In the present embodiment, a second liquid carrier 14 used is a structure having the shape of a container which can retain the second liquid 4 or through which the second liquid 4 can flow. A first liquid carrier 12 used is a structure having the shape of a container which can retain the first liquid 2 or through which the first liquid 2 can flow. The first liquid carrier 12 includes a bottom that serves as a support 8 allowing movement of the first liquid 2 and the second liquid 4. Cells 10 are retained on the lower surface of the support 8. The first liquid carrier 12 may be accommodated in a cavity of the second liquid carrier 14.

As show in FIG. 13, the first liquid carrier 12 in which cells are retained on the support 8 is placed in the second liquid carrier 14 retaining the second liquid 4, and the first liquid is retained in the first liquid carrier 12. Thereby the support 8 may be provided at the interface 6 between the first liquid 2 and the second liquid 4 and cells 10 may be provided on the support 8.

When cells 10 are cultured in the embodiment, nutritional components are supplied to cells 10 while cells 10 are in contact with the first liquid 2 through the support 8. The cells 10 are also exposed to the second liquid 4. Thereby it is possible to effectively evaluate the action of a non-hydrophilic substance in the second liquid 4 on the cells 10 and a sheet-shaped cell structure.

In the third and fourth embodiments illustrated in FIG. 12 and FIG. 13, cells 10 are retained and cultured on the upper surface and the lower surface of the support 8, respectively. However, the embodiments are not limited thereto. For example, cells 10 may be retained and cultured on the lower surface and the upper surface of the support 8 as far as the support 8 allows movement of the second liquid 4 and the interface 6 is maintained.

In the third and fourth embodiments, the first liquid carrier 12 and the second liquid carrier 14 may respectively be in various forms within the range that can secure the liquid-liquid interface therebetween and the contact with the two liquids. Namely, the first liquid carrier 12 and the second liquid carrier 14 including the support 8 may use an inner surface or an outer surface of a side wall thereof, rather than the bottom thereof, as a support 8 to retain and cultivate cells 10. The first liquid carrier 12 and the second liquid carrier 14 retaining cells 10 may be configured so that the whole structures thereof serve as supports 8, or partial structures thereof serve as supports.

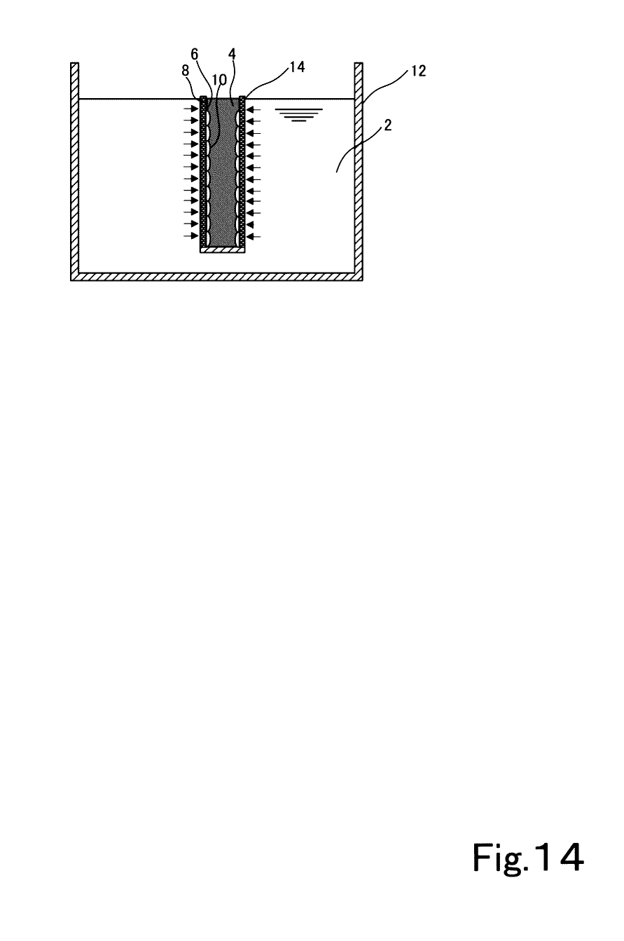

For example, as shown in FIG. 14 which is a modification of the third embodiment, the second liquid carrier 14 may be a structure having the shape of an elongated tube or container. A side wall of the second liquid carrier 14 may serve as a support 8 allowing movement of a first liquid, thereby cells 10 are retained and cultured at the inner surface of the first liquid carrier 12. Movement of the second liquid 4 is prevented by cell-cell junctions of cells 10 in the support 8 and by the material of the second liquid carrier 14 per se in other sites.

In the modification cells 10 are cultured on the inner surface of the support 8 which is the side wall of the second liquid carrier 14. Nutritional components are supplied to the cells 10 from the first liquid 2 through the support 8 and the cells 10 are exposed to the second liquid 4. According to the modification, it is possible to effectively evaluate the action of a non-hydrophilic substance on the cells 10 and a tubular cell structure.

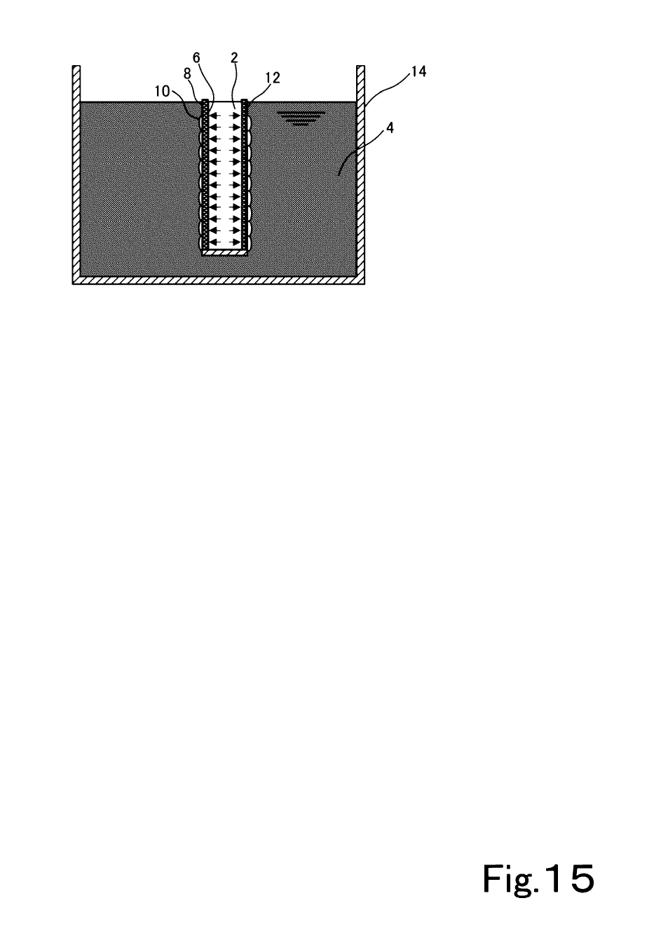

Similarly, as shown in FIG. 15 which is a modification of the fourth embodiment, the side wall of a tubular first liquid carrier 12 is used as a support 8 allowing movement of a first liquid 2, in order to retain cells 10 while cells 10 are in contact with a second liquid 4 at an outer surface of the side wall. Similarly to FIG. 14, the second liquid 4 is prevented to move at the support 8 and other sites of the first liquid carrier 14. Thereby it is possible to effectively evaluate the action of a non-hydrophilic substance on the cells 10 and a tubular cell structure.

As described above, according to the present evaluation method, an action of a non-hydrophilic substance on cells 10 may be evaluated and an action thereof on a structure of cells 10 having any three-dimensional shape. When cells 10 form a structure, an effect of a non-hydrophilic substance may vary according to the strength of cell adhesion that is required for the maintenance of the structure or intercellular stress from the effect thereof to a structure having a general sheet shape. Therefore, the present evaluation method allows practical evaluation of the action of a non-hydrophilic substance on cells.

When the present cultivation step is carried out, the order of bringing cells 10 into contact with the first liquid 2 and the second liquid 4 is not particularly limited as far as the contact with the liquids intended by the present disclosure is maintained. The contacts may be carried out according to the order described in the above embodiments or other orders, or may be carried out simultaneously.

When the present cultivation step is carried out, it is preferable to prepare a first liquid carrier 12 or a second liquid carrier 14 in which cells 10 are retained in a support 8. A pre-cultivation step for preparing such a liquid carrier may be for example carried out as follows.

First, cells 10 are inoculated at a predetermined site of a support 8 in a first liquid carrier 12 or a second liquid carrier 14 and the cells 10 are cultured under the condition generally applied to the cells 10. The culture may be or may not be continued until the cells 10 cover the whole region of the support 8; however, by culturing until the cells 10 densely cover the whole region, movement of the second liquid 4 may be prevented, allowing effectively carrying out the present cultivation step.

The present cultivation step may be carried out by applying culture conditions (temperature, gas, medium, humidity, etc.) suitable for cells 10 to be used. Particularly, the first liquid 2 is preferably a medium suitable for cells 10.

(Evaluation Step)

The present evaluation method may include a step of evaluating the action of the non-hydrophilic substance on the thus cultured cells 10. The cells 10 exposed to the second liquid 4 containing the non-hydrophilic substance are subjected to the action of the non-hydrophilic substance while receiving a supply of nutritional components from the first liquid 2 and maintaining the activity. Thus the present cultivation step mimics or reproduces in vivo exposure of cells 10 to a foreign substance, which is a non-hydrophilic substance.

The action of the non-hydrophilic substance on the cells 10 may be evaluated by, for example, measuring the viability of the cells 10 according to various methods. The action may alternatively be evaluated by measuring the transepithelial electric resistance (TER) described hereinabove.

The viability of cells may be measured according to various well-known methods. Examples of the methods include, in addition to electric methods in which, for example, the membrane potential is measured, biochemical methods such as color developing methods in which the death of cells is detected using blue tetrazolium and the like and observation methods such as microscopy. A person skilled in the art can select an appropriate method among those well-known methods to apply to the present evaluation step.

As described above, according to the present evaluation method, it is possible to expose cells to a foreign substance while supplying a nutritional component or the like to the cells. Namely, it is possible to evaluate the action of a non-hydrophilic substance on cells while mimicking or reproducing the in vivo situation of the cells.

According to the present evaluation method, a nutritional component is supplied to cells, and thus it is possible to evaluate the action of a non-hydrophilic substance over a prolonged period of time.

Further, according to the present evaluation method, cells may be exposed to a non-hydrophilic substance that has been conventionally difficult to be evaluated by dissolving or dispersing the non-hydrophilic substance in a solvent of a second liquid that is a non-hydrophilic liquid. Thereby the action of the non-hydrophilic substance on cells may be essentially and accurately evaluated.

Due to the above reasons, the present evaluation method is practical for evaluating the action of a non-hydrophilic substance on cells.

(Cell-Containing Structure)

The present disclosure provides a cell-containing structure. The present structure includes a first liquid carrier, a second liquid carrier; a support that is disposed in the vicinity of an interface between a first liquid and a second liquid and through which either or both of the first liquid and the second liquid can move; and a cell retained in at least a part of the support while contacting the first liquid and the second liquid.

According to the structure, cells are ensured to be in contact with both the first liquid and the second liquid. According to the cell structure, the activity of cells may be effectively maintained while bringing the cells into contact with the second liquid. Thus the structure is useful for evaluation of a non-hydrophilic substance by utilizing a second liquid that is a non-hydrophilic liquid. In addition, the structure is useful for evaluation of the action of a non-hydrophilic substance on a cell structure having a desired three-dimensional shape.

For the present structure, various embodiments of the first liquid, the second liquid, the first carrier, the second carrier and the support described in relation to the present evaluation method may be applied as they are. In the various embodiments described hereinabove, the first liquid and the second liquid are described to be retained or to flow through; however it is not necessary for the present structure to retain or allow flow of the first liquid and the second liquid.

(Evaluation Device)

The present disclosure also provides a device for evaluating an action of a non-hydrophilic substance on a cell. The present device may include a first liquid carrier; a second liquid carrier; and a support that is disposed in the vicinity of an interface between a first liquid and a second liquid, through which either or both of the first liquid and the second liquid can move and that can retain a cell.

According to the evaluation device, cells are ensured to be in contact with both the first liquid and the second liquid by being retained in the support, and thus the structure may be constituted that is useful for evaluation of a non-hydrophilic substance by utilizing a second liquid that is a non-hydrophilic liquid.

For the present evaluation device, various embodiments of the first liquid, the second liquid, the first carrier, the second carrier and the support described in relation to the present evaluation method may be applied as they are. In the various embodiments described hereinabove, the first liquid and the second liquid are described to be retained or to flow through and cells are retained; however, the present evaluation device do not require those features as essential.

(Method for Screening a Cytocompatible or Biocompatible Non-Hydrophilic Substance)

The present disclosure also provides a method for screening a cytocompatible non-hydrophilic substance. The screening method of the present disclosure may include a step of culturing a cell while the cell is in contact with a first liquid and a second liquid containing a non-hydrophilic substance by using a support through which either or both of the first liquid and the second liquid can move as a scaffold at an interface between the first liquid and the second liquid, and a step of evaluating an action of the non-hydrophilic substance on the cell. The method allows evaluation of compatibility of the non-hydrophilic substance to the cell on the basis of the action.

According to the method, as described hereinabove, it is possible to mimic or reproduce the contact of cells in vivo with a foreign substance that is a non-hydrophilic substance, and thus a practical cytocompatible or biocompatible non-hydrophilic substance may be screened.

In the present method, various embodiments of the first liquid, the second liquid, the first carrier, the second carrier and the support described in relation to the present evaluation method as well as various embodiments of the cultivation step and the evaluation step of the present evaluation method may be applied.

According to the present evaluation method, compatibility of toxicity of a non-hydrophilic substance to cells may be evaluated on the basis of the action of the non-hydrophilic substance on the cells. On the basis of the evaluation result, a cytocompatible or biocompatible non-hydrophilic substance may be screened. The present screening method allows screening of a toxic non-hydrophilic substance. Thus the present screening method may be carried out as a method for screening a cytotoxic non-hydrophilic substance.

### EXAMPLES

The present invention is specifically described by referring to Examples according to the present disclosure. The following Examples merely describe the present disclosure without limiting thereof.

### Example 1

In the present Example, in order to determine a HSP serving as an index of biocompatibility (cytotoxicity) to a cell, various test liquids indicated below were directly brought into contact with cells without a medium and a cytotoxicity test (WST-8 assay) was carried out. S1 to S21 denote test liquids and P1 to P4 denote comparative liquids generally known to have strong toxicity,

TABLE 11

| ID | Solvent | CAS No. | SMILES or Formula |
|---|---|---|---|
| S1 | 1,3-Bis(Trifluoromethyl) | 402-31-3 | C1═CC(═CC(═C1)C(F)(F)F)C(F)(F)F |
| | Benzene | | |
| S2 | Perfluorohexane, PFC 5060 | 355-42-0 | FC(F)(C(F)(C(F)(C(F)(C(F)(C(F)(F)F)F)F)F)F)F |
| S3 | 1,1,2,2-Tetrafluoroethyl | 40 [symbol] - | FC(CCC(F)(F)F)(C(F)F)F |
| | 2,2,2-Trifluoroethyl | 7 [symbol] -0 | |
| | Ether, Ae-3000 | | |
| S4 | 1H,1H,7H-Dodecafluoro-1- | 33 [symbol] - | FC(C(F)(F)C(F)(F)CO)(F)C(F)(F)C(F)(F)C(F)F |
| | heptanol | 99- [symbol] | |
| S5 | HFE-7100 | 183702- | COC(C(C(C(F)(F)F)(F)F)(F)F)(F)F |
| | | 07-8 | |
| S6 | HFE-7200 | 163702- | CCOC(F)(F)C(F)(F)C(F)(F)C(F)(F)(F), |
| | | 05-5, | CCOC(F)(F)C(C(F)(F)(F))(F)C(F)(F)(F) |
| | | 163702- | |
| | | 05-4 | |
| S7 | HFE-7300 | 132182- | FC(F)(C(F)(F)C(F)(F)C(F)(F)C(F)(F)F)C(F)(F)OC |
| | | 92-4 | |
| S8 | GALDEN HT55 | Mixture | 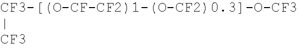 |
| S9 | GALDEN HT80 | Mixture | 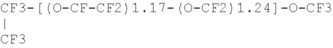 |
| S10 | GALDEN HT110 | Mixture | 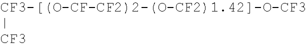 |
| S11 | GALDEN HT135 | Mixture | 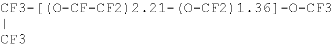 |
| S12 | GALDEN HT170 | Mixture | 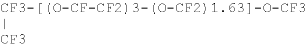 |
| S13 | GALDEN HT200 | Mixture | 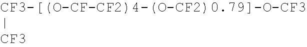 |
| S14 | GALDEN HT230 | Mixture | 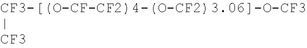 |
| S15 | GALDEN HT270 | Mixture | 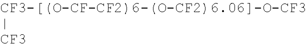 |
| S16 | OS-10 | 107-48-0 | C[Si](C)(C)O[Si](C)(C)C |
| | (Hexamethyldisiloxane) | | |
| S17 | OS-20 | 107-51-7 | C[Si](C)(C)O[Si](C)(C)O[Si](C)(C)C |
| | (Octamethyltrisiloxane) | | |
| S18 | OS-30 | 141-62-6 | C[Si](C)(C)O[Si](C)(C)O[Si](C)(C)O[Si](C)(C)C |
| | (Decamethyltetrasiloxane) | | |
| S19 | Decamethylcyclopentasiloxane | 541-02-6 | [Si]1(C)(C)O[Si](C)(C)O[Si](C)(C)O[Si](C)(C)O[Si](O1)(C)C |
| S20 | 1-Bromoperfluorooctane | 423-55-2 | FC(F)(F)C(F)(F)C(F)(F)C(F)(F)C(F)(F)C(F)(F)C(F)(F)C(F)(F)Br |
| S21 | HFE-7000 | 375-03-1 | COC(F)(C(F)(C(F)(F)F)F)F |
| P1 | Water | 7732-18- | [H]O[H] |
| | | 5 | |
| P2 | Ethanol | 64-17-5 | CCO |
| P3 | Acetone | 67-64-1 | CC(C)═O |
| P4 | Diethyl ether | 80-29-7 | CCOCC |

[symbol]

indicates data missing or illegible when filed

Human airway epithelial cell line BEAS-2B was inoculated into a 96-well plate at 1×10⁵ /cm². A medium used was 10% FBS-containing RPMI-1640.

After 24 hours, the medium was removed, test liquids were respectively added to directly bring each test liquid and the cells into contact for about 2 hours. During the contact the well plate was sealed to prevent evaporation. Thereafter, the medium was replaced with 0.5% FBS-containing RPMI-1640. After 24 hours from the completion of the contact with the test liquid, the cytotoxicity test (WST-8 assay) was carried out acceding to the standard procedures. The results are shown in Table 12.

TABLE 12

| | | Normalized | Standard | HSP distance | | | | | |
|---|---|---|---|---|---|---|---|---|---|
| ID | Solvent | cell | error | from core | δD | δP | δH | Mvol | Tot |
| S1 | 1,3-Bis(Trifluoromethyl)Benzene | 0.01 | 0.005 | 10.2 | 17 | 6.8 | 0 | 155.2 | 18.3 |
| S2 | Perfluorohexane, PFC 5060 | 0.28 | 0.041 | 4.4 | 12.1 | 0 | 0 | 201.2 | 12.1 |
| S3 | 1,1,2,2-Tetrafluoroethyl 2,2,2-Trifluoroethyl Ether, A₆-3000 | 0.56 | 0.042 | 3.6 | 14 | 4.9 | 3.9 | 136 | 15.3 |
| S4 | 1H,1H,7H-Dodecafluoro-1-heptanol | 0.01 | 0.003 | 5.5 | 13.6 | 4.6 | 8.1 | 195 | 15.5 |
| S5 | HFE-7100 | 1.04 | 0.023 | 3.1 | 13.7 | 2.2 | 1 | 164.5 | 13.9 |
| S6 | HFE-7200 | 0.88 | 0.023 | 1.7 | 13 | 2.9 | 2 | 182 | 13.5 |
| S7 | HFE-7300 | 0.92 | 0.035 | 1.4 | 12.8 | 2.4 | 2.1 | 217 | 13.2 |
| S8 | GALDEN HT55 | 0.92 | 0.05 | 2.5 | 12.1 | 4.2 | 2.2 | 202 | 13 |
| S9 | GALDEN HT80 | 0.91 | 0.038 | 3.2 | 11.6 | 4.7 | 2.5 | 249 | 12.9 |
| S10 | GALDEN HT110 | 0.25 | 0.025 | 4.4 | 10.9 | 4.4 | 2.1 | 329 | 12 |
| S11 | GALDEN HT135 | 1.18 | 0.062 | 4.7 | 10.7 | 4.3 | 2 | 344 | 11.7 |
| S12 | GALDEN HT170 | 1.05 | 0.106 | 6.2 | 9.9 | 4.3 | 1.8 | 424 | 10.9 |
| S13 | GALDEN HT200 | 1.18 | 0.081 | 7.4 | 9.3 | 3.7 | 1.1 | 482 | 10 |
| S14 | GALDEN HT230 | 1.05 | 0.022 | 8.8 | 8.6 | 4.9 | 2 | 561 | 10.1 |
| S15 | GALDEN HT270 | 0.5 | 0.055 | — | — | — | — | — | — |
| S16 | OS-10 (Hexamethyldisiloxane) | 0 | 0.004 | 3.5 | 12.6 | 2 | 0 | 212.4 | 12.8 |
| S17 | OS-20 (Octamethyltrisiloxane) | 0.05 | 0.018 | 3.7 | 12.2 | 1.8 | 0 | 288.4 | 12.3 |
| S18 | OS-30 (Decamethyltetrasiloxane) | 0.92 | 0.069 | 4 | 11.7 | 2.4 | 0 | 363.8 | 11.9 |
| S19 | Decamethylcyclopentasiloxane | 0.92 | 0.072 | 2.7 | 12.9 | 1.3 | 1 | 388.7 | 13 |
| S20 | 1-Bromoperfluorooctane | 1.2 | 0.051 | 2.2 | 12.9 | 1.9 | 1.3 | 261 | 13.1 |
| S21 | HFE-7000 | 1.01 | 0.042 | 3.1 | 13 | 4.2 | 1 | 141.9 | 13.7 |
| P1 | Water | 0.01 | 0.004 | 41.5 | 15.5 | 18 | 42.3 | 18 | 47.8 |
| P2 | Ethanol | 0.01 | 0.004 | 18.3 | 15.8 | 8.8 | 19.4 | 58.6 | 26.5 |
| P3 | Acetone | 0.01 | 0.004 | 10.4 | 15.5 | 10.4 | 7 | 73.8 | 19.9 |
| P4 | Diethyl ether | 0.01 | 0.004 | 3.5 | 14.5 | 2.9 | 4.6 | 104.7 | 15.5 |

| | | | | | |
|---|---|---|---|---|---|
| Anti-cytotoxic | δDAC | δPAC | δHAC | R | Limit |
| HSP value: | 12.73 | 2.33 | 3.48 | 3.4 | 0.7 |

Table 12 shows the results of the cytotoxicity test indicated with normalized cell survival rates and standard deviations (1σ). The normalized cell survival rate is a value obtained by dividing a cell survival rate of a test liquid by a cell survival rate of a control liquid, which is 0.5% FBS-containing RPM-1640, with the cell survival rate of the control liquid being 1. In the present Example, a test liquid having a normalized cell survival rate of 0.7 or more was identified as cytocompatible.

Table 12 also shows, in addition to HSPs (δD, δP, δH) of test liquids obtained with the software described above, molar volumes and Hildebrand solubility parameters (Tat) obtained with the software. Table 11 further shows HSP distances from the HSP core (anti-cytotoxic (biocompatible) liquid) based on the above-mentioned data. The values indicated with italics are empirically calculated values using the software. The hyphen indicates that empirical calculation was impossible because of lack of data in the database and issues in molecular weight and the like.

(1) Normalized Cell Survival Rate and Molar Volume

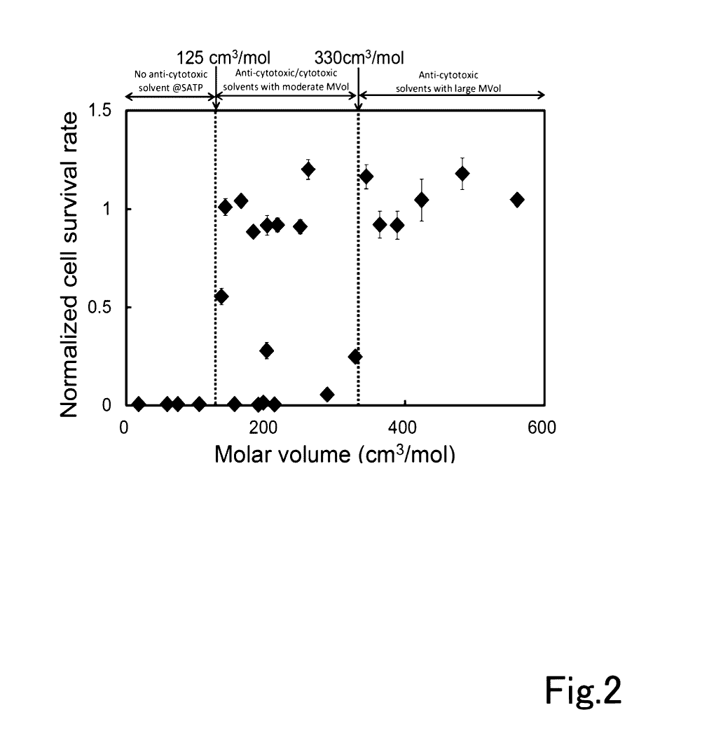

FIG. 2 shows the relationship between the molar volume and the normalized cell survival rate of test liquids. As shown in FIG. 2, there was a certain relationship between the magnitude of molar volume and the normalized cell survival rate. Namely, when the molar volume is 125 cm³/mol or less, the normalized cell survival rate was extremely low (almost no biocompatibility), when the molar volume was more than 125 cm³/mol and less than 330 cm³/mol, the normalized cell survival rate was distributed from low to high, and when the molar volume was 330 cm³/mol or more, the normalized cell survival rate was generally high. Accordingly, it was found that biocompatibility of a liquid is affected by the molar volume per se of the liquid, and when the molar volume is 125 cm³/mol or less, the liquid per se is strongly toxic (low biocompatibility), when the molar volume is 330 cm³/mol or more, the liquid is low toxic (high biocompatibility) and when the molar volume is more than 125 cm³/mol and less than 330 cm³/mol, cytotoxicity (biocompatibility) exhibited significantly depends on the HSP.

(2) Calculation of HSP Core of Biocompatible Liquid

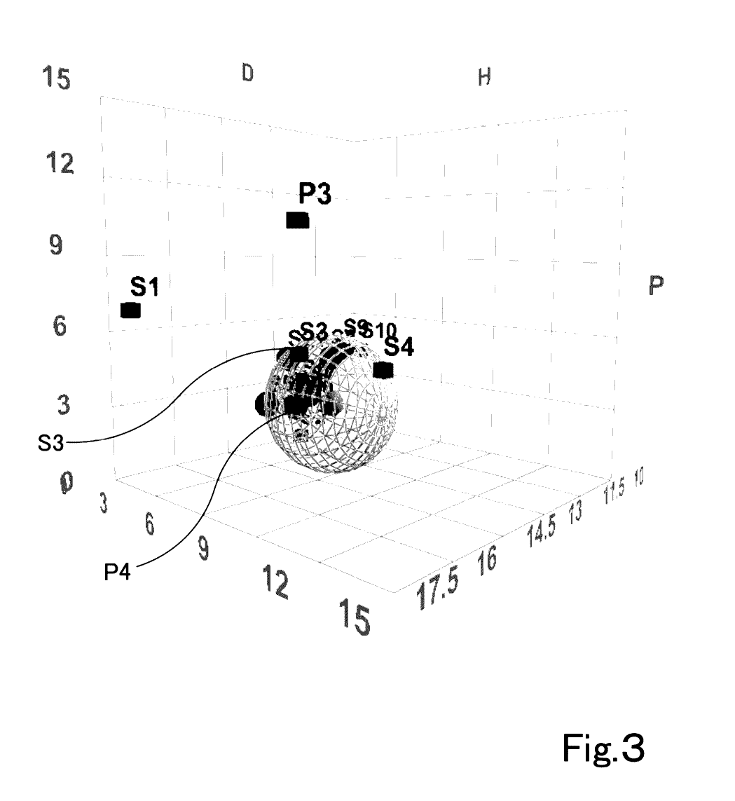

According to the above (1), it was found that, with regard to the relationship between biocompatibility and HSP, when the molar volume is less than 330 cm³/mol, HSP affects the normalized cell survival rate (biocompatibility). Thus, amongst test liquids fulfilling the range of the molar volume, test liquids having a normalized cell survival rate of 0.7 or more were selected and a HSP core s calculated a HSP core was calculated from the HSPs according to the Hansen sphere method. The results are shown in a part of Table 12. Table 12 also shows the HSP distances D of test liquids and comparative liquids from the obtained HSP core. Further in FIG. 3, HSPs and the HSP core based on the HSPs of test liquids respectively having a molar volume of less than 330 cm³/mol and a normalized cell survival rate of 0.7 or more and HSPs of cytotoxic liquids.

As shown in Table 12 and FIG. 3, the HSP core (δD, δP, δH) based on the HSPs of test liquids respectively having a molar volume of less than 330 cm³/mol and a normalized cell survival rate of 0.7 or more was (12.73, 2.33, 3.46). As apparent tom FIG. 3, HSPs of biocompatible liquids exist around the HSP core and HSPs of cytotoxic liquids exist away from the HSP core. In FIG. 3, test liquids S5 to S9, S20 and S21 were within the interaction radius R (3.4) from the HSP core.

(3) Normalized Cell Survival Rate and HSP Distance

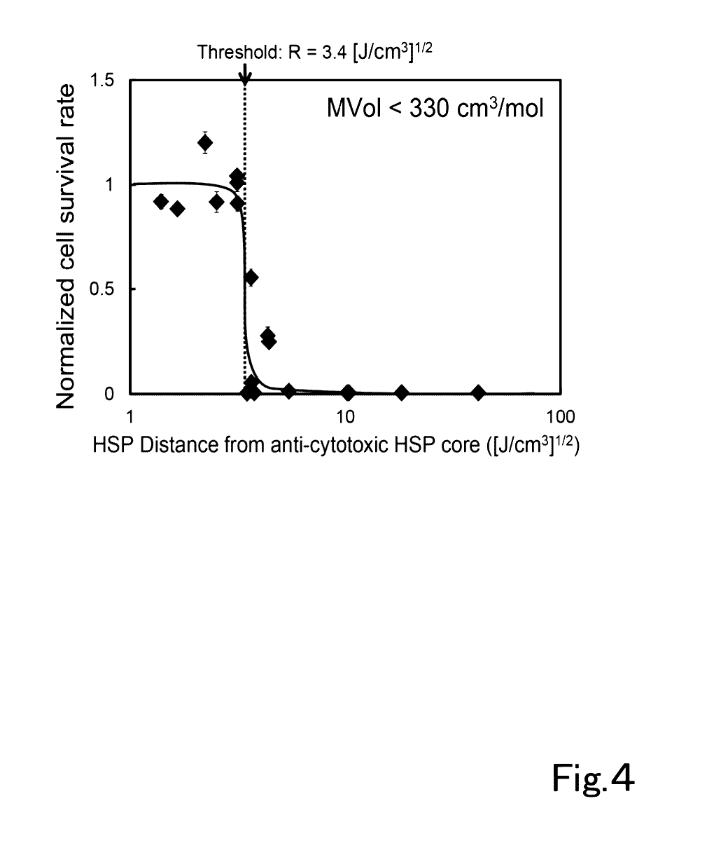

With regard to the test liquids having a molar volume of less than 330 cm³/mol, a plot of the normalized cell survival rate and a distance D from the HSP core is shown in FIG. 4. As shown in FIG. 4, it was found that when the HSP distance D is equal or more than 3.4 ([J/cm³]^{1/2}), the normalized cell survival rate is drastically decreased. The distance D also corresponded to the HSP distance D corresponding to the normalized cell survival rate of 0.7 that was used to define biocompatibility. Namely, the HSP distance D may serve as a threshold for defining an interaction radius R from a HSP core.

Accordingly, the HSP sphere serving as HSP threshold information could be defined. The HSP sphere is shown as a frame sphere in FIG. 3. As shown in FIG. 3, it was found that HSPs of test liquids which are biocompatible liquids (normalized cell survival rate of 0.7 or more) are included in the HSP sphere.

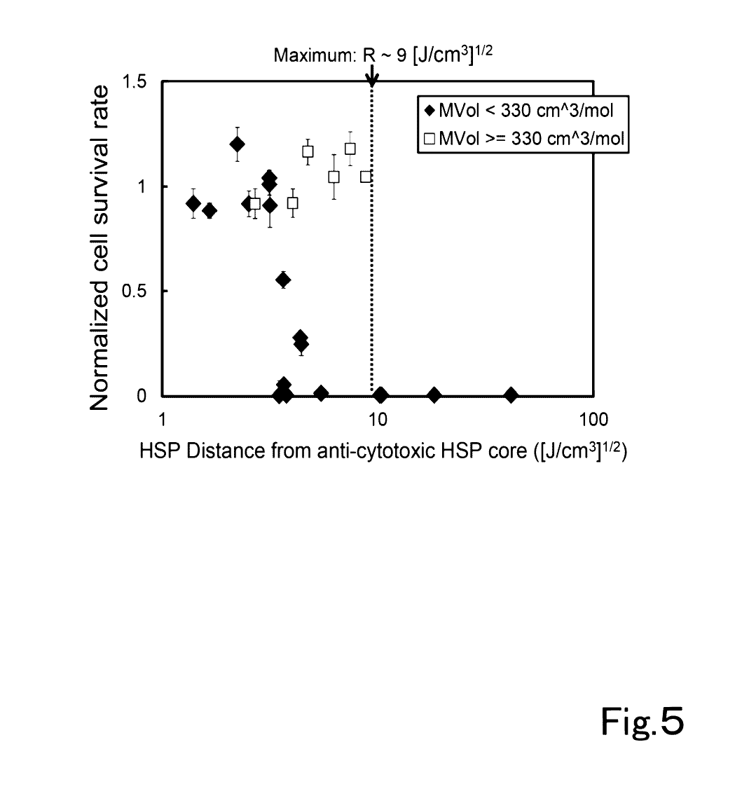

With regard to the test liquids having a molar volume of 330 cm³/mol or more, a plot of the normalized cell survival rate and a distance D from the HSP core is shown in FIG. 5. As shown in FIG. 5, there was no biocompatible test liquid having the HSP distance D of more than 9.0 ([J/cm³]^{1/2}). Thus, the HSP distance D may serve as a threshold for defining an interaction radius R from a HSP core.

Accordingly, two HSP spheres could be defined according to the molar volume serving as HSP threshold information. For the HSP sphere with regard to the molar volume of less than 330 cm³/mol, a liquid is not biocompatible when a HSP thereof is not within the HSP sphere having an interaction radius R of 3.4 ([J/cm³)]^{1/2}) from the HSP core. On the other hand, it was found that for the HSP sphere with regard to the molar volume of 330 cm³/mol or more, a liquid may be biocompatible when a HSP thereof is within the HSP sphere having an interaction radius R of 9.0 ([J/cm³]^{1/2}) from the HSP core.

(4) List of Biocompatible Liquids

Biocompatible liquids that can be defined by two HSP spheres as defined above are shown in Tables 3 to 5. Thus, 39 biocompatible liquids (candidates) could be selected that were defined by the following HSP sphere 1, as shown in Table 3. Further, 181 biocompatible liquids (candidates) could be selected that were defined by the following HSP sphere 2, as shown in Table 4A, Table 4B, Table 5A and Table 5B.

The liquids were selected on condition of melting point of less than 25° C. and boiling point of above 33° C.

HSP sphere 1:

Core (δD, δP, δH): (12.73, 2.33, 3.46) ([J/cm³]^{1/2})

Interaction radius R: 9.0 ([J/cm²]^{1/2})

HSP sphere 2:

Core (δD, δP, δH): (12.73, 2.33, 3.46) ([J/cm³]^{1/2})

Interaction radius R: 3.4 ([J/cm³]^{1/2})

### Example 2

In the present Example, as a different approach from Example 1, cytotoxicity of a liquid was predicted a priori. For this purpose, it was believed to be sufficient to take HSP values of cell components into account, and thus HSPs of main substances (C1 to C10) (for those with hydrophobic moieties and hydrophilic moieties, the moieties are separately indicated), as shown in the following Table, that constitute cells were obtained with the software described above. With regard to cholesterol, DNA and water (1 wt % miscible), HSPs were obtained from the database of the software and HSPs for other substances were obtained as empirically calculated values using the software. The interaction radius R employed for the components other than water, cholesterol and DNA were 5.0 [J/cm³]^{1/2}.

TABLE 13

| ID | Cell constituent | SMILES | δD | δP | δH | R |
|---|---|---|---|---|---|---|
| C1 | Cholesterol | CC(C)CCCC(C)C1CCC2C1(CCC3C2CC═C4C3(CCC(C4)O)C)C | 20.4 | 2.8 | 9.4 | 12 |
| C2 | DNA | — | 19 | 30 | 11 | 11 |
| C3 | Cell membrane hydrophobic | CCCCCCCCCCCCCCCCC | 16 | 0 | 0 | 5 |
| | portion#1: Phosphatidylcholine | | | | | |
| | hydrophobic portion#1 | | | | | |
| C4 | Cell membrane hydrophobic | CCCCCCCC═CCCCCCCCC | 16 | 1.3 | 1.8 | 5 |
| | portion#2: Phosphatidylcholine | | | | | |
| | hydrophobic portion#2 | | | | | |
| C5 | Cell membrane hydrophobic | CCCC═CCC═CCC═CCC═CCCCCCC | 17 | 0.9 | 2.4 | 5 |
| | portion#3: Phosphatidylethanolamine | | | | | |
| | hydrophobic | | | | | |
| C6 | Cell membrane hydrophilic | COP(═O)(═O)OCC(OC(═O)C)COC═OC | 17 | 12 | 12 | 5 |
| | portion#1: Phosphatidylcholine | | | | | |
| | hydrophilic portion | | | | | |
| C7 | Cell membrane hydrophilic | C[N+](C)(C)CCOP([O—])(═O)OCC(OC(═O)C)COC(═O)C | 17 | 9.8 | 16 | 5 |
| | portion#2: Sphingomyelin | | | | | |
| | hydrophilic portion | | | | | |
| C8 | Cell membrane hydrophilic | C[N+](C)(C)CCOP([O—])(═O)OCC(NC(═O)C)C(O)C | 18 | 17 | 20 | 5 |
| | portion#3: Phosphatidylethanolamine | | | | | |
| | hydrophilic portion | | | | | |
| C9 | Cell membrane hydrophilic | O═C(O[C@@H](COP(O)(═O)OC[C@H](N)C(O)═O)COC(═O)CC)C [symbol] | 18 | 13 | 19 | 5 |
| | portion#4: Phosphatidylserine | | | | | |
| C10 | Water (1% soluble) | [H]O[H] | 15.1 | 17.1 | 16.9 | 18.1 |

[symbol]

indicates data missing or illegible when filed

(1) Relative Energy Differences Between HSPs of Biocompatible Liquids and HSP Spheres of Cell Components

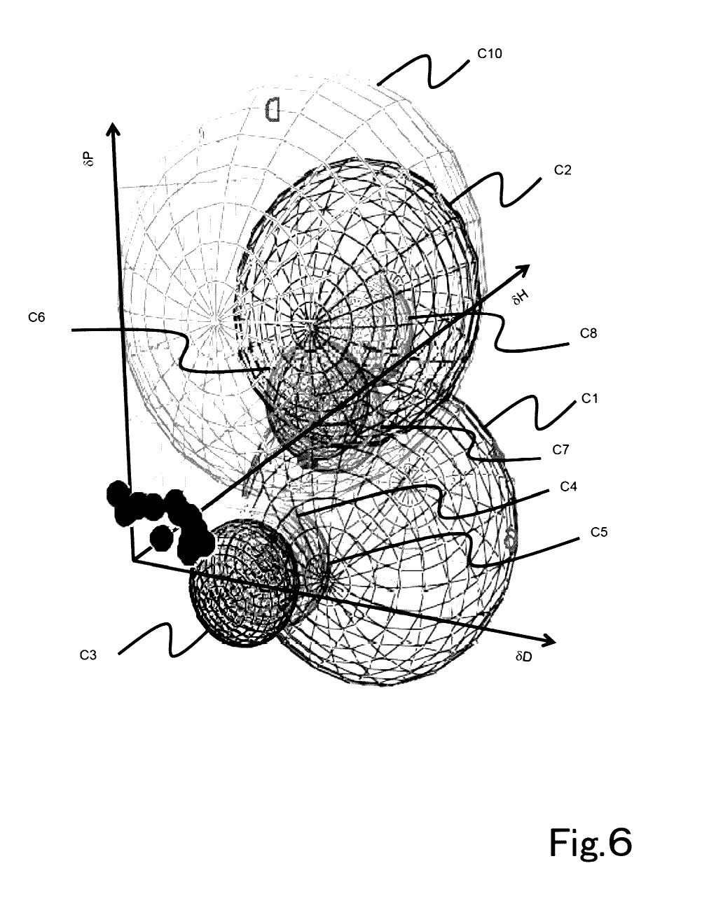

Among the test liquids show in Table 12, only cytocompatible liquids (S5 to 59, S11 to S14 and S18 to S21) were selected, and relative energy differences (REDs) between HSPs thereof and HSP spheres of cell components shown in Table 13 were calculated and shown in Table 14. The relationship between HSPs of biocompatible liquids and each HSP sphere of cell components is shown in FIG. 6.

TABLE 14

| | RED | RED | RED | RED | RED | RED | RED | RED | RED | RED |
|---|---|---|---|---|---|---|---|---|---|---|
| | to C1 | to C2 | to C3 | to C4 | to C5 | to C6 | to C7 | to C8 | to C9 | to C10 |
| S5 | 1.3 | 2.9 | 1.0 | 1.0 | 1.3 | 3.3 | 3.7 | 5.1 | 4.4 | 1.2 |
| S6 | 1.4 | 2.8 | 1.4 | 1.3 | 1.6 | 3.2 | 3.6 | 5.0 | 4.3 | 1.2 |
| S7 | 1.4 | 2.9 | 1.4 | 1.3 | 1.7 | 3.3 | 3.6 | 5.1 | 4.3 | 1.2 |
| S8 | 1.5 | 2.8 | 1.8 | 1.7 | 2.0 | 3.2 | 3.6 | 5.0 | 4.3 | 1.1 |
| S9 | 1.6 | 2.8 | 2.0 | 1.9 | 2.2 | 3.2 | 3.6 | 5.0 | 4.3 | 1.1 |
| S11 | 1.7 | 2.9 | 2.3 | 2.2 | 2.6 | 3.6 | 4.0 | 5.3 | 4.6 | 1.2 |
| S12 | 1.9 | 3.0 | 2.6 | 2.6 | 2.9 | 3.9 | 4.3 | 5.5 | 4.9 | 1.2 |
| S13 | 2.0 | 3.1 | 2.8 | 2.8 | 3.1 | 4.2 | 4.5 | 5.8 | 5.2 | 1.3 |
| S14 | 2.1 | 3.1 | 3.1 | 3.1 | 3.4 | 4.2 | 4.6 | 5.7 | 5.1 | 1.3 |
| S18 | 1.6 | 3.0 | 1.7 | 1.8 | 2.2 | 3.8 | 4.2 | 5.6 | 4.9 | 1.3 |
| S19 | 1.4 | 3.0 | 1.2 | 1.3 | 1.6 | 3.5 | 3.9 | 5.3 | 4.6 | 1.3 |
| S20 | 1.4 | 2.9 | 1.3 | 1.3 | 1.6 | 3.4 | 3.8 | 5.2 | 4.5 | 1.2 |
| S21 | 1.4 | 2.7 | 1.4 | 1.4 | 1.7 | 3.2 | 3.6 | 5.0 | 4.3 | 1.2 |

As shown in Table 14, the relative energy differences between. HSPs of biocompatible liquids and HSP spheres of cell components were all 1.0 or more. Namely, this shows that the biocompatible liquids are liquids that “do not infiltrate into cell components” and “do not dissolve cell components”.

As shown in FIG. 6, in the HSP space, the biocompatible HSP space is the space outside of the HSP spheres of the respective cell components. It is apparent that biocompatible liquids have HSPs that are outside of the HSP spheres.

(2) List of Biocompatible Liquids

In total, 241 biocompatible liquids (candidates) having HSPs within the biocompatible HSP space defined as above were selected as shown in Tables 7 to 10. The liquids were selected on condition of melting point of less than 25° C. and boiling point of above 33° C.

### Example 3

In the present Example, biocompatibility of Novec 7200 and Novec 7300, which were selected as biocompatible liquid candidates in Examples 1 and 2, on plants was examined by using leaves of Arabidopsis thaliana. A plant (a leaf of A. thaliana, about 1 cm×5 mm, a 1.5-ml tube) was soaked in each of these organic solvents or ethanol, methanol or acetone which are organic solvents apparently initiating cytotoxicity, and the plant was observed for any change in the appearance after 2 hours. Further, the leaves used for the test after observation were soaked in water over 16 hours followed by observation. Water was used as a control. Although water is cytotoxic to naked cells, it is not cytotoxic to cells having cell walls, and thus could be used as a control.

NOVEC 7200 and NOVEC 7300, which are organic solvents identified as biocompatible liquid candidates in cultured cells, did not change an appearance of leaves of A. thaliana, such as dead leaves. On the other hand, ethanol, methanol and acetone, which are apparently cytotoxic in cultured cells, eroded epidermal cells of the leaves of A. thaliana and caused effusion of chlorophyll.

In addition, two biocompatible liquid candidates did not cause changes in an appearance to leaves of A. thaliana after the observation compared to a control, water. On the other hand, ethanol, methanol and acetone caused atrophy (deformation) of leaves of A. thaliana, and thus the leaves lost original forms thereof and had decreased extent of green color.

Accordingly, it was found that cytocompatible liquids identified by the present method exhibit cytocompatibility to wide range of cells including not only animal cells but also plant cells.

### Example 4

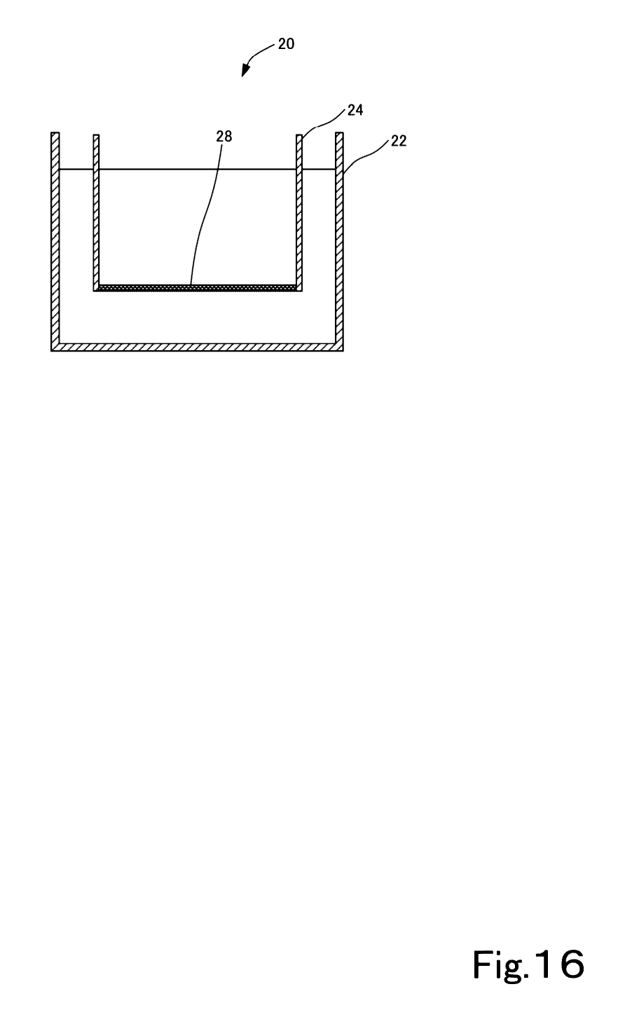

In the present Example, an action of non-hydrophilic liquids having the fluorocarbon structure was evaluated using human airway epithelial cells Calu-3. As a device for evaluation, the evaluation device 20 illustrated in FIG. 16 was used. As shown in FIG. 16, the device 20 includes wells 22 having cavities opening upwards and inserts 24, and is configured to be able to accommodate the inserts 24 in the wells 22. At the bottom of each insert, a porous support 28 is provided that can retain and cultivate cells. The support 28 allows movement of water, which is a solvent of a cell growth medium.

The growth medium corresponds to the first liquid of the present disclosure and the non-hydrophilic liquid having the fluorocarbon structure corresponds to the second liquid of the present disclosure. The well 22 corresponds to the first liquid cavity of the present disclosure, the insert 24 corresponds to the second liquid cavity of the present disclosure and the support 28 corresponds to the support of the present disclosure.

Cells were inoculated on each support 28 in the insert 24 and the space in the well 22 other than the insert 24 and the cavity in the insert 24 were filled with the cell growth medium. The evaluation device 20 was then left to stand in a CO₂ incubator for a few days to allow dense growth of cells on the whole upper surface of the support 28, thereby carrying out the pre-cultivation step. In the pre-cultivation step, the transepithelial electric resistance (TER) was measured over time to confirm that a barrier (coverage and functional tight junctions) was constructed on the surface of the cells that is in contact with the insert 24.

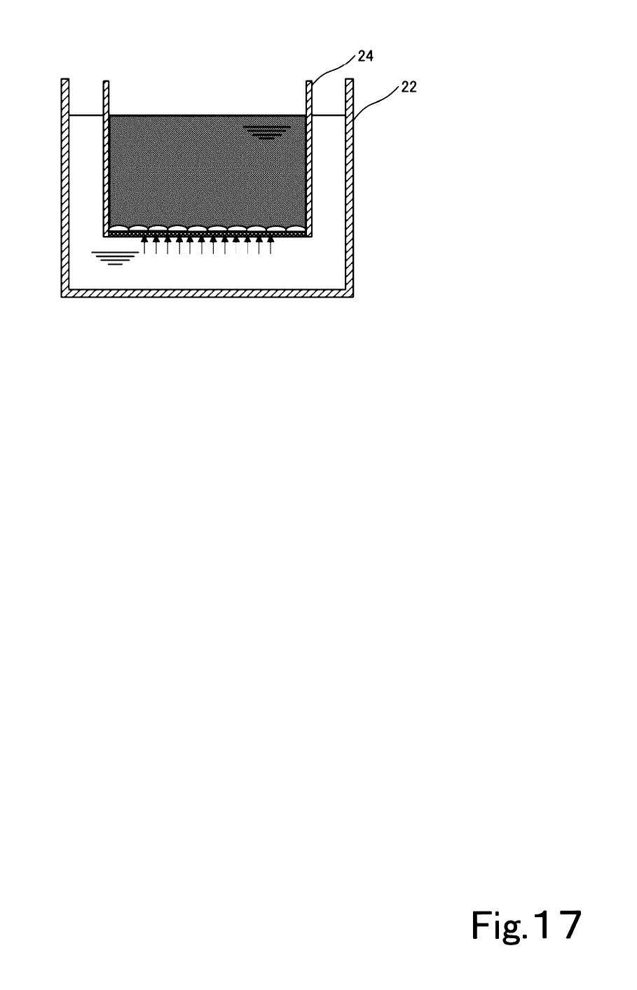

Next, a cell structure illustrated in FIG. 17 was prepared. Namely, the growth medium in the insert 24 was removed and 200 ul (microliter) of C₆F₆ (perfluorohexane) was added so that cells were brought into contact with perfluorohexane. Under this situation, the device was left to stand in the CO₂ incubator for 3 hours to carry out cultivation. Thereafter, perfluorohexane, C₆F₆, was removed from the cavity of the insert 24, 20 ul of fresh growth medium was added and cultivation was carried out in the CO₂ incubator for 24 hours.

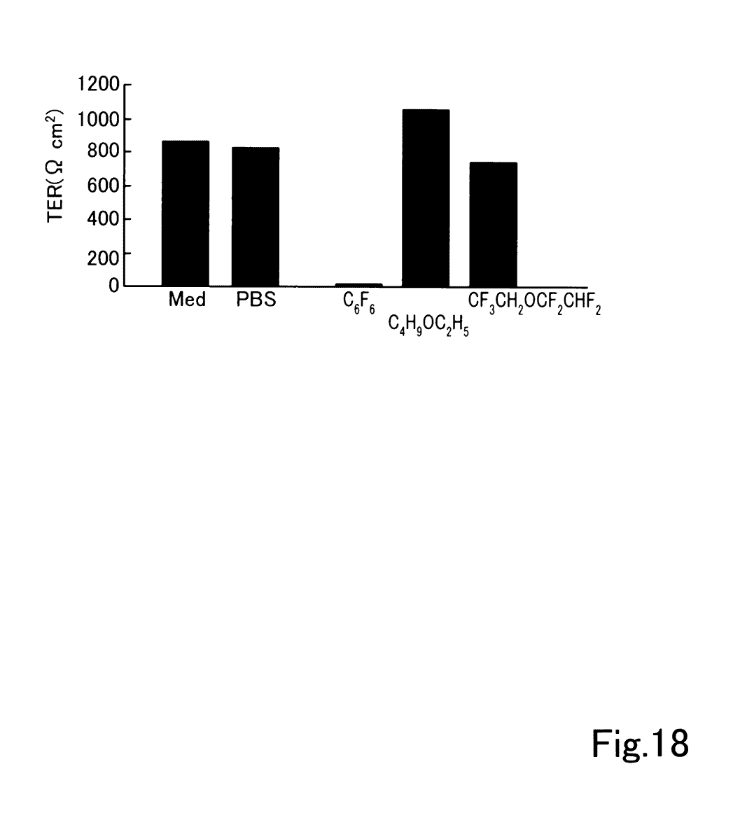

After 24 hours, the cells on the support 28 in the cell structure removed from the CO₂ incubator were again measured for the transepithelial electric resistance (TER) after removal of the growth medium in order to confirm the extent of formation of the cell barrier. The results are shown in FIG. 18.

In the similar manner as above except that two hydrofluoroethers (C₄F₉OC₂H₅ and CF₉CF₂OCF₂CHF₂) were used instead of perfluorohexane, the extent of formation of the cell barrier was examined after contact with those liquids. As controls, hydrophilic liquids, MED (culture medium) and phosphate buffered saline (PBS), were used and the TER was measured in the similar manner. The results are shown in FIG. 12.

An increase in the TER means better intercellular tight junctions, while a decrease in the TER means cell death or disruption of intercellular tight junctions.

As shown in FIG. 18, perfluorohexane significantly promoted cell death and the like, while two hydrofluoroethers (C₄F₉OC₂H₅ and CF₉CF₂OCF₂CHF₂) had less effect on cell death or intercellular tight junctions than perfluorohexane, which effect was almost the same as the controls.

Accordingly, it was found that non-hydrophillic liquids having the fluorocarbon structure have significantly different actions on cells depending on the structures and compositions thereof. It was also found that there are non-hydrophilic liquids having the fluorocarbon structure that are as cytocompatible as PBS. Further, it was found that hydrofluoroethers could be screened as biocompatible non-hydrophilic liquids.

### Example 5

In the present Example, hydrofluoroether (C₄F₉OC₂H₅) was used as the non-hydrophilic liquid and perfluorooctanoic acid (PFOA) was used as the non-hydrophilic substance. Solutions of PFOA in hydrofluoroether having various PFOA concentrations (0, 0.01, 1, 1 and 10 g/L) were prepared. The solutions correspond to the second liquid of the present disclosure.

In the similar manner as Example 4 except that the solutions of PFOA in hydrofluoroether were used, the procedure was carried out up to the pre-cultivation step. Thereafter the cultivation step was carried out by using the solutions as the second liquid and leaving the device in the CO₂ incubator for 15 hours.

Thereafter perfluorohexane, C₆F₆, was removed from the cavity of the insert 24, 200 μl of an aqueous solution of a medium containing WST-8 (2-(2-methoxy-4-nitrophenyl)-3(4-nitrophenyl)-5-(2,4-disulfophenyl)-2H-tetrazolium), which is a kind of a tetrazolium salt, was added and the device was let to stand in the CO₂ incubator for 20 minutes for cultivation.

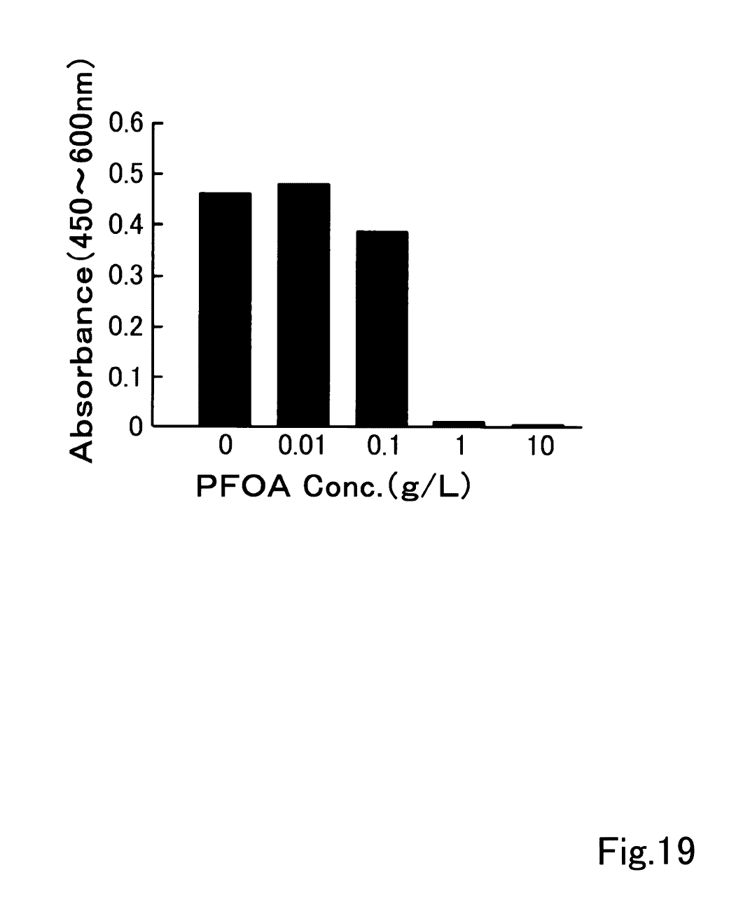

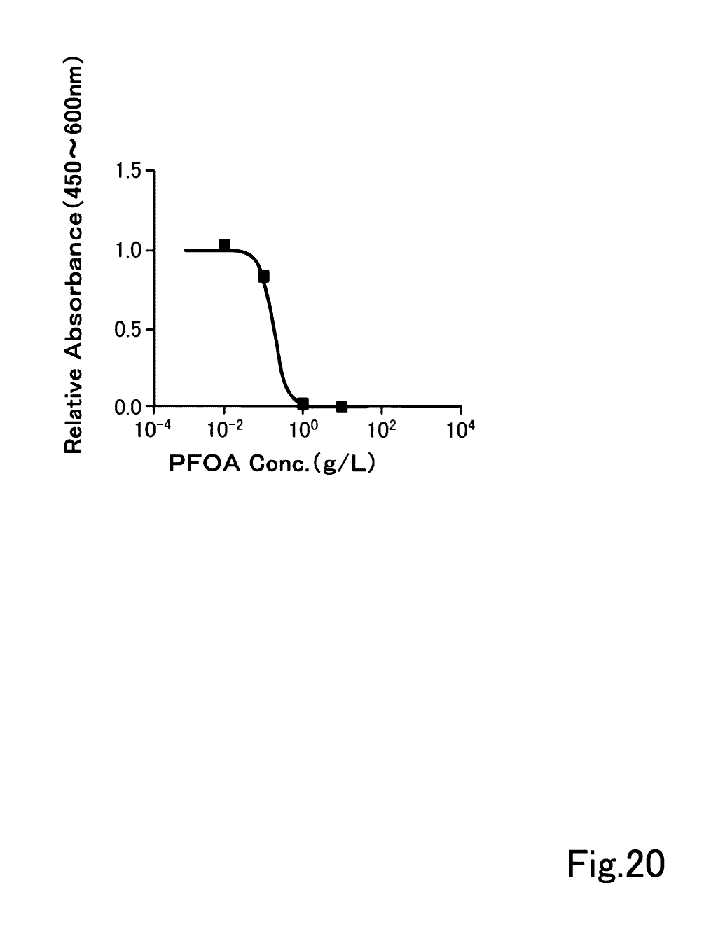

Thereafter 100 μl of the medium was transferred from the insert 24 of the cell structure into a microplate and the absorbance at the wavelengths 450 nm and 600 nm was measured. The results are shown in FIG. 19 and FIG. 20.

FIG. 19 shows the relationship between the PFOA concentration and the absorbance. FIG. 20 shows the relative absorbance at various PFOA concentrations with the absorbance at the PFOA concentration of 0 being 1. As shown in FIG. 19 and FIG. 20, the absorbance decreased as the PFOA concentration decreased and the absorbance was almost at the same level until the PFOA concentration was 0.1 g/L, while the absorbance was hardly detected at or above 1 g/L of PFOA. Thus, it was found that when the PFOA concentration was at or above 1 g/L, PFOA had an action of causing cell death.

From the above results, it was found that even a water-insoluble substance such as PFOA may be safely evaluated for the biocompatibility (toxicity) thereof in vitro using cells in a concentration-dependent manner when a biocompatible non-hydrophilic liquid was used as a solvent. It was also found that by using a non-hydrophilic liquid, even a substance which is non-hydrophilic and may have high cytotoxicity such as PFOA may be appropriately evaluated for cytocompatibility or biocompatibility (toxicity) thereof.

1. A method for screening or determining a biocompatible liquid, comprising:
a step of identifying whether or not a test liquid has a Hansen solubility parameter (HSP) compatible to a cell to be tested,
wherein the compatible Hansen solubility parameter (HSP) is determined on the basis of threshold information of a Hansen solubility parameter associated with biocompatibility obtained on the basis of one or more liquids for which a level of biocompatibility to the cell has been established and Hansen solubility parameters of the liquids.

2. The method according to claim 1, wherein the compatible Hansen solubility parameter (HSP) is present within a HSP sphere defined by a predetermined core (δD, δP, δH) in a Hansen solubility parameter (HSP) space based on Hansen solubility parameters of one or more liquids biocompatible to the cell and by a predetermined interaction radius R.

3. The method according to claim 2, wherein the compatible Hansen solubility parameter (HSP) is present outside of a HSP sphere defined by a predetermined core (δD, δP, δH) in a Hansen solubility parameter (HSP) space based on Hansen solubility parameters of one or more cell components of the cell and by a predetermined interaction radius.

4. The method according to claim 3, wherein the compatible Hansen solubility parameter (HSP) is present within a HSP sphere having a core (δD, δP, δH) of (12.73, 2.33, 3.46) ([J/cm³]^{1/2}) and an interaction radius R of 3.4 ([J/cm³]^{1/2}) or less.

5. The method according to claim 3, wherein the compatible Hansen solubility parameter (HSP) is present within a HSP sphere having a core (δD, δP, δH) of (12.73, 2.33, 3.46) ([J/cm³]^{1/2}) and an interaction radius R of 9.0 ([J/cm³]^{1/2}) or less.

6. The method according to claim 4, wherein the compatible Hansen solubility parameter (HSP) is present outside of all HSP spheres of DNA, cholesterol, water, phosphatidylcholine, sphingomyelin, phosphatidylethanolamine and phosphatidylserine.

7. The method according to claim 5, wherein the Hansen solubility parameter (HSP) is present outside of all HSP spheres of DNA, cholesterol, water, phosphatidylcholine, sphingomyelin, phosphatidylethanolamine and phosphatidylserine.

8. The method according to claim 1, further comprising a step of identifying whether or not the test liquid has a molar volume compatible to the cell,
wherein the compatible molar volume is determined on the basis of threshold information of a liquid molar volume associated with biocompatibility obtained on the basis of one or more liquids for which a level of biocompatibility to the cell has been established and molar volumes of the liquids.

9. The method according to claim 8, wherein:
the compatible molar volume is above 125 cm³/mol and less than 330 cm³/mol; and
the compatible Hansen solubility parameter (HSP) is present within a HSP sphere having a core (δD, δP, δH) of (12.73, 2.33, 3.46) ([J/cm³]^{1/2}) and an interaction radius R of 3.4 ([J/cm³]^{1/2}) or less.

10. The method according to claim 9, wherein:
the compatible molar volume is 330 cm³/mol or more; and
the Hansen solubility parameter (HSP) is present within a HSP sphere having a core (δD, δP, δH) of (12.73, 2.33, 3.46) ([J/cm³]^{1/2}) and an interaction radius R of 9.0 ([J/cm³]^{1/2}) or less.

11. The method according to claim 9, wherein the compatible Hansen solubility parameter (HSP) is present outside of all HSP spheres of DNA, cholesterol, water, phosphatidylcholine, sphingomyelin, phosphatidylethanolamine and phosphatidylserine.

12. The method according to claim 10, wherein the compatible Hansen solubility parameter (HSP) is present outside of all HSP spheres of DNA, cholesterol, water, phosphatidylcholine, sphingomyelin, phosphatidylethanolamine and phosphatidylserine.

13. The method according to claim 1, wherein the biocompatible liquid has a boiling point of above 33° C. and a melting point of less than 25° C.

14. The method according to claim 1, which is a method for screening a liquid biocompatible to a naked cell devoid of an extracellular component.

15. A cell-containing structure comprising:
a first liquid carrier through which a first liquid that is a hydrophilic liquid can flow or which can retain the first liquid;
a second liquid carrier through which a second liquid that is a non-hydrophilic liquid immiscible with the first liquid and contains a substance which is dissolved or uniformly dispersed in the non-hydrophilic liquid can flow or which can retain the second liquid;
a support through which either or both of the first liquid and the second liquid can move; and
a cell retained in at least a part of the support while contacting the first liquid and the second liquid.

16. The structure according to claim 15, which is a device for evaluating an action of the substance on the cell.

17. A method for evaluating an action of a substance on a cell, comprising the steps of:
culturing the cell while the cell is in contact with a first liquid that is a hydrophilic liquid, and a second liquid that is a non-hydrophilic liquid immiscible with the first liquid and contains the substance which is dissolved or uniformly dispersed in the non-hydrophilic liquid, by using a support through which either or both of the first liquid and the second liquid can move as a scaffold in the vicinity of an interface between the first liquid and the second liquid; and
evaluating the action of the non-hydrophilic substance on the cell.

18. A method for screening a cytocompatible substance, comprising:
culturing a cell while the cell is in contact with a first liquid that is a hydrophilic liquid, and a second liquid that is a non-hydrophilic liquid immiscible with the first liquid and contains the substance which is dissolved or uniformly dispersed in the non-hydrophilic liquid, by using a support through which either or both of the first liquid and the second liquid can move as a scaffold at an interface between the first liquid and the second liquid, and
evaluating an action of the substance on the cell,
wherein the cytocompatibility of the substance is evaluated on the basis of the action.
